(19) United States
(12) Patent Application Publication
(10) Pub. No.: US 2025/0177567 A1
(43) Pub. Date: Jun. 5, 2025

(54) MODIFIED CRISPR-BASED GENE EDITING SYSTEM AND METHODS OF USE

(71) Applicant: GENEDITBIO LIMITED, Hong Kong (CN)

(72) Inventors: Zongli ZHENG, Hong Kong (CN); Bang WANG, Hong Kong (CN); Miao YU, Hong Kong (CN)

(21) Appl. No.: 18/843,775

(22) PCT Filed: Mar. 8, 2023
(86) PCT No.: PCT/CN2023/080356

§ 371 Date: Sep. 4, 2024

Related U.S. Application Data
(60) Provisional application No. 63/318,362, filed on Mar. 9, 2022.

(51) Int. Cl. A61K 48/00; C12N 9/22; C12N 15/11; C12N 15/88; C12N 15/90

(52) U.S. Cl. CPC A61K 48/005; C12N 9/22; C12N 15/111; C12N 15/88; C12N 15/907; C12N 2310/20; C12N 2310/3519; C12N 2310/531

(57) ABSTRACT

Disclosed herein are systems comprising one or more modified single-guide RNAs (sgRNAs) and a donor DNA, wherein each of the modified sgRNAs comprises one or more internal anchors that are at least 5 nucleotides away from both 3′ and 5′ ends of each of the modified sgRNAs, wherein the donor DNA comprises one or more binding segments capable of binding to an internal anchor of the one or more internal anchors. Further disclosed herein are methods of using the systems described here

### CROSS-REFERENCE TO RELATED APPLICATIONS

This application is a U.S. National Phase Application under 35 U.S.C. 371 of International Application No. PCT/CN2023/080356, filed on Mar. 8, 2023, which claims the benefit of U.S. Provisional Application Ser. No. 63/318,362 filed on Mar. 9, 2022, the entirety of which is hereby incorporated by reference herein.

### STATEMENT AS TO FEDERALLY SPONSORED RESEARCH

The disclosure was made with government support under Grant No. 2016-02830 awarded by the Swedish Research Council to Zongli Zheng.

### SEQUENCE LISTING

This application contains references to amino acid sequences and/or nucleic acid sequences which have been submitted concurrently herewith as the sequence listing file entitled “000211usnp_SequenceListing.XML”, file size 1,421,263 bytes, created on Sep. 10, 2024. The aforementioned sequence listing is hereby incorporated by reference in its entirety pursuant to 37 C.F.R. § 1.52 (e) (5).

### BACKGROUND

Gene editing technologies have been blooming and are powerful tools to manipulate genetic materials in target cells, tissues, or organisms. Clustered regularly interspaced short palindromic repeats (CRISPR)-related technologies are among the most promising gene editing tools available. However, a low rate of desired homology directed repair precise (HDR)-mediated editing, a less satisfactory off-target rate, and a high translocation rate and its resulting mutagenesis have become the main obstacles in further advancing the technologies and its wider usage.

### SUMMARY

In one aspect, to address the need for a more effective and specific gene editing outcome, provided herein is a system for altering a target sequence, comprising a modified single-guide RNA (sgRNA) and a donor DNA, wherein the modified sgRNA comprises a CRISPR RNA (crRNA) and a trans-active RNA (tracrRNA), wherein the modified sgRNA comprises one or more internal anchors that are at least 5 nucleotides away from both 3′ and 5′ ends of the modified sgRNA, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI).

In some embodiments, the non-covalent bond is a Watson-Crick interaction.

In some embodiments, the modified sgRNA comprises a nexus, a first hairpin, and a single-stranded region between the tracrRNA and the crRNA. In some embodiments, the modified sgRNA further comprises a bulge region. In some embodiments, the modified sgRNA further comprises a second hairpin.

In some embodiments, the internal anchor of the one or more internal anchors is located in a single-stranded region of the modified sgRNA. In some specific embodiments, the internal anchor of the one or more internal anchors is located in the single-stranded region between the tracrRNA and the crRNA. In other specific embodiments, the internal anchor of the one or more internal anchors is located in a single-stranded region within the first hairpin. In other specific embodiments, the internal anchor of the one or more internal anchors is located in a single-stranded region between the nexus and the first hairpin. In other specific embodiments, the modified sgRNA further comprises a second hairpin, and wherein the single-stranded region is within the second hairpin.

In some embodiments, each of the one or more internal anchors or each of the one or more binding segments is 3-nucleotide to 100-nucleotide long. In other embodiments, each of the one or more internal anchors or each of the one or more binding segments is 3-nucleotide to 20-nucleotide long. In yet other embodiments, each of the one or more internal anchors or each of the one or more binding segments is about 5-nucleotide long.

In some embodiments, each of the one or more internal anchors comprises a sequence from SEQ ID NOs. 1 to 472 from Table 1. In other embodiments, each of the one or more internal anchors comprises a sequence from SEQ ID NOs. 473 to 3056 from Table 2. In other embodiments, each of the one or more binding segments comprises a sequence from SEQ ID NO. 3057 to 3528 from Table 3. In other embodiments, each of the one or more binding segments comprises a sequence from SEQ ID NO. 3529 to 6112 from Table 4.

In some embodiments, the one or more binding segments are linked by a linker. In some specific embodiments, the linker is about 1 to 30-nucleotide long. In other specific embodiments, the linker is about 10 to 25-nucleotide long. In other embodiments, the linker is a sequence of poly-deoxyadenosines.

In some embodiments, the SOI comprises the target sequence with one or more nucleotide substitution, one or more nucleotide insertion, one or more nucleotide deletion, or any combination thereof. In some embodiments, the one or more nucleotide insertion comprises 1 to 100 nucleotides. In some embodiments, the one or more nucleotide insertion comprises 101 to 1,000 nucleotides. In some embodiments, the one or more nucleotide insertion comprises 1,001 to 10,000 nucleotides. In some embodiments, the one or more nucleotide insertion comprises 1,001 to 10,000 nucleotides. In some embodiments, the one or more nucleotide insertion comprises 10,001 to 100,000 nucleotides. In other embodiments, the one or more nucleotide insertion comprises 2 to 10 random nucleotides. In other embodiments, the one or more nucleotide deletion comprises 1 to 50 nucleotides.

In some embodiments, the second portion of the donor DNA further comprises an upstream and/or a downstream homology arm. In specific embodiments, the upstream homology arm is 5 to 1000-nucleotide long. In specific embodiments, the downstream homology arm is about 10 to 1000-nucleotide long. In other embodiments, the upstream homology arm is 100 to 1,000-nucleotide long. In other embodiments, the downstream homology arm is about 41 to 1,000-nucleotide long.

In some embodiments, the first portion of the donor DNA is at 5′ of the second portion of the donor DNA. In other embodiments, the first portion of the donor DNA is at 3′ of the second portion of the donor DNA.

In some embodiments, the donor DNA is single-stranded. In other embodiments, the first portion of the donor DNA is single-stranded and the second portion of the donor DNA is fully or partially double-stranded.

In some embodiments, the donor DNA is close ended on 3′ and/or 5′ end.

In some embodiments, the system further comprises a CRISPR nuclease. In specific embodiments, the CRISPR nuclease is a DNA nuclease. In specific embodiments, the DNA nuclease is a Cas9, a Cas12, a Cas14, or a CasΦ.

In another aspect, provided herein is a system comprising a donor DNA and two modified single-guide RNAs (sgRNAs) for cutting at a first locus on a first chromosome and a second locus on a second chromosome, wherein each of the modified sgRNAs comprises a CRISPR RNA (crRNA) and a trans-active RNA (tracrRNA), wherein each of the modified sgRNAs comprises one or more internal anchors that are at least 5 nucleotides away from both 3′ and 5′ ends of each of the modified sgRNAs, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI), wherein the donor DNA comprises an upstream homology arm and/or a downstream homology arm.

In some embodiments, the first chromosome and the second chromosome are the same. In some specific embodiments, the first locus is at 5′ of the second locus. In other embodiments, the first chromosome and the second chromosome are different. In some embodiments the two modified sgRNAs target different locus on the same gene. In some embodiments, the two modified sgRNAs target different locus on the same strand. In some embodiments, the two modified sgRNAs target different locus on the different strands. In some embodiments, the system comprising a second donor DNA. In some embodiments, the donor DNA and the second donor DNA each comprises an upstream homology arm and/or a downstream homology arm. In some embodiments, the homology arm(s) of the donor DNA and the homology arm(s) of the second donor DNA are complementary to sequences on the same strand. In some embodiments, the homology arm(s) of the donor DNA and the homology arm(s) of the second donor DNA are complementary to sequences on the different strands.

In some embodiments, the first locus and the second locus are at least 50, 100, 1,000, 10,000, or 100,000 nucleotides apart.

In some embodiments, the upstream homology arm flanks 5′ end of the first locus. In other embodiments, the downstream homology arm flanks 3′ end of the second locus.

In some embodiments, the non-covalent bond is a Watson-Crick interaction.

In some embodiments, the modified sgRNA comprises a nexus, a first hairpin, and a single-stranded region between the tracrRNA and the crRNA. In some embodiments, the modified sgRNA further comprises a bulge region. In some embodiments, the modified sgRNA further comprises a second hairpin.

In some embodiments, the internal anchor of the one or more internal anchors is located in a single-stranded region of the modified sgRNA. In some specific embodiments, the internal anchor of the one or more internal anchors is located in the single-stranded region between the tracrRNA and the crRNA. In other specific embodiments, the internal anchor of the one or more internal anchors is located in a single-stranded region within the first hairpin. In other specific embodiments, the internal anchor of the one or more internal anchors is located in a single-stranded region between the nexus and the first hairpin. In other specific embodiments, the modified sgRNA further comprises a second hairpin, and wherein the single-stranded region is within the second hairpin.

In some embodiments, each of the one or more internal anchors or each of the one or more binding segments is 3-nucleotide to 100-nucleotide long. In other embodiments, each of the one or more internal anchors or each of the one or more binding segments is 3-nucleotide to 20-nucleotide long. In yet other embodiments, each of the one or more internal anchors or each of the one or more binding segments is about 5-nucleotide long.

In some embodiments, each of the one or more internal anchors comprises a sequence from SEQ ID NOs. 1 to 472 from Table 1. In other embodiments, each of the one or more internal anchors comprises a sequence from SEQ ID NOs. 473 to 3056 from Table 2. In other embodiments, each of the one or more binding segments comprises a sequence from SEQ ID NO. 3057 to 3528 from Table 3. In other embodiments, each of the one or more binding segments comprises a sequence from SEQ ID NO. 3529 to 6112 from Table 4.

In some embodiments, the one or more binding segments are linked by a linker. In some specific embodiments, the linker is about 1 to 30-nucleotide long. In other specific embodiments, the linker is about 10 to 25-nucleotide long. In other embodiments, the linker is a sequence of poly-deoxyadenosines.

In some embodiments, the SOI comprises a region between the first locus and the second locus with one or more nucleotide substitution, one or more nucleotide insertion, one or more nucleotide deletion, or any combination thereof. In some embodiments, the one or more nucleotide insertion comprises 1 to 100 nucleotides. In some embodiments, the one or more nucleotide insertion comprises 101 to 1,000 nucleotides. In some embodiments, the one or more nucleotide insertion comprises 1,001 to 10,000 nucleotides. In some embodiments, the one or more nucleotide insertion comprises 1,001 to 10,000 nucleotides. In some embodiments, the one or more nucleotide insertion comprises 10,001 to 100,000 nucleotides. In some embodiments, the one or more nucleotide deletion comprises 1 to 100 nucleotides. In some embodiments, the one or more nucleotide deletion comprises 101 to 1,000 nucleotides. In some embodiments, the one or more nucleotide deletion comprises 1,001 to 10,000 nucleotides. In some embodiments, the one or more nucleotide deletion comprises 1,001 to 10,000 nucleotides. In some embodiments, the one or more nucleotide deletion comprises 10,001 to 100,000 nucleotides.

In specific embodiments, the upstream homology arm is 5 to 1000-nucleotide long. In specific embodiments, the downstream homology arm is about 10 to 1000-nucleotide long. In other embodiments, the upstream homology arm is 100 to 1,000-nucleotide long. In other embodiments, the downstream homology arm is about 41 to 1,000-nucleotide long.

In some embodiments, the first portion of the donor DNA is at 5′ of the second portion of the donor DNA. In other embodiments, the first portion of the donor DNA is at 3′ of the second portion of the donor DNA.

In some embodiments, the donor DNA is single-stranded. In other embodiments, the first portion of the donor DNA is single-stranded and the second portion of the donor DNA is fully or partially double-stranded.

In some embodiments, the donor DNA is close ended on 3′ and/or 5′ end.

In some embodiments, the system further comprises a CRISPR nuclease. In specific embodiments, the CRISPR nuclease is a DNA nuclease. In specific embodiments, the DNA nuclease is a Cas9, a Cas12, a Cas14, or a CasΦ.

In another aspect, provided herein is a method of modifying a cell, wherein the method comprises transporting a system as described herein.

In some embodiments, the transporting comprises: (a) incubating the CRISPR nuclease and the modified sgRNA to form a ribonucleoprotein (RNP) complex; (b) applying the donor DNA to the RNP complex; and (c) delivering the RNP complex-donor DNA from (b) to the cell. In some specific embodiments, in step (a) the ratio of the CRISPR nuclease and the modified sgRNA is about 1:0.5 to about 1:10. In other specific embodiments, in step (a) the ratio of the CRISPR nuclease and the modified sgRNA is about 1:1 to 1:2.

In other embodiments, the transporting comprises: (a) providing one or more vectors comprising a nucleotide sequence encoding the CRISPR nuclease and a nucleotide sequence encoding the modified gRNA; (b) delivering the one or more vectors of (a) to the cell; and (c) delivering the donor DNA to the cell. In specific embodiments, step (c) is performed about 6 to 48 hours after step (b).

In some embodiments, the delivering is achieved by viral vectors, liposomes, lipid nanoparticles, or electroporation.

In some embodiments, the cell is an immune cell. In specific embodiments, the immune cell is a T cell, a B cell, an NK cell, or a hematopoietic stem cell.

In some embodiments, the method is performed ex vivo or in vivo the method is performed ex vivo or in vivo.

In some embodiments, a percentage of desired editing is at least 10%, at least 50%, at least 100%, or at least 200% higher than a comparable system without the donor DNA comprising a first portion that binds to the modified sgRNA and/or without the modified sgRNA with the one or more internal anchors. In other embodiments, the method has an off-target rate at least 10%, at least 50%, or at least 100% lower than a comparable system without the donor DNA comprising the first portion that binds to the modified sgRNA and/or without the modified sgRNA with the one or more internal anchors. In other embodiments, the method has a translocation, large insertion, or large deletion rate at least 10%, at least 50%, or at least 100% lower than a comparable system without the donor DNA comprising the first portion that binds to the modified sgRNA and/or without the modified sgRNA with the one or more internal anchors.

In another aspect, provided herein is a method of treating a genetic disorder, wherein the method comprises administering to a subject with an effective amount of the system as described herein. In some embodiments, the SOI comprises a sequence that reverses or alleviate the genetic disorder.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-1C show negligible editing efficiency of CRISPR Cas9 system with an RNA-DNA fusion-oligo design as the single guide (referred to as the “Fusion-Oligo design”). (FIG. 1A) A schematic presentation of the Fusion-Oligo design. Cas9, colored in yellow, is positioned with the target double-strand DNA, where both strands contain nicked gaps representing the normal editing outcome of Cas9. The RNA-DNA fusion oligo contains a 5′ portion RNA spacer sequence complementary to the bottom target DNA strand, a middle region containing trans-activating RNA (tracrRNA) sequence (together the RNA part is also known as single-guide RNA [sgRNA], colored in red), and a 3′ portion of donor DNA, colored in blue thick line, serves the template for homology directed reparation (HDR; containing an ‘AAG’ insertion for a desired ‘CTT’ insertion on the target DNA). (FIG. 1B) Efficiency of the desired editing outcome (‘CTT’ insertion at the target position) by three experiment groups: the previously developed Prime Editor 2 (PE2; plasmid purchased from AddGene 132775, by Andrew V. Anzalone, Nature, 2019 [PMID: 31634902]) serving as a comparison group, the fusion RNA-DNA guide design (the ‘Test’ group with different lengths of the donor DNA, 14, 17 and 20 nt), and a no-Cas9 control group with the fusion RNA-DNA guide design (N.C. group with different lengths of the donor DNA, 14, 17, 20 and 23 nt). (FIG. 1C) Efficiency of all editing, including the desired (‘CTT’ insertion at the target position) and undesired (other insertions and deletions) by the same three experiment groups as in (FIG. 1B).

FIGS. 2A-2C illustrate that gene editing efficiency of CRISPR Cas9 system with Watson-Crick base pairing between an RNA tail, at the terminal end of the sgRNA, and a DNA tail at one end of the donor DNA template (referred to as “Terminal-Anchor design”). (FIG. 2A) A schematic presentation of the Terminal-Anchor design. Cas9, colored in yellow, is positioned with the target double-strand DNA, where both strands contain nicked gaps representing the normal editing outcome of Cas9. The sgRNA, colored in red, contains an extended 3′ portion that is complementary to and extended 5′ portion of the donor DNA. (FIG. 2B) Efficiency of the desired editing outcome (‘CTT’ insertion at the target position) by different experiment groups: no Cas9 control with the Terminal-Anchor design sgRNA (the first 4 samples in the ‘Negative’ group with different lengths of the donor DNA tail of 3, 7, 10 and 13 bases); no-Cas9 and no sgRNA (the 5th to 7th samples in the ‘Negative’ group with different lengths of the donor DNA tail of 7, 10 and 13 bases); the test group using the Terminal-Anchor design with a Watson-Crick base pairing of length of 0, 3, 5, 7, 10, and 13 bases). (FIG. 2C) Efficiency of all editing, including the desired (‘CTT’ insertion at the target position) and undesired (other insertions and deletions) by the same experiment groups as in (FIG. 2B).

FIGS. 3A-3E show various designs of donor DNAs and their relative positions to target DNA, and guide RNA. (FIG. 3A) A schematic presentation of a CRISPR gene editing system with Watson-Crick base pairing between an RNA sequence, which is located at the internal part of a guide RNA, and a DNA portion that is part of a donor DNA template (referred to as “Internal Anchored design”). Cas9, colored in yellow, is positioned with the target double-strand DNA, where both strands contain nicked gaps representing the normal editing outcome of Cas9. The single-guide RNA (sgRNA), colored in red, contains an internal 3′ portion that is complementary to a portion of DNA sequence in the donor DNA. (FIG. 3B) The Internal-Anchored design with a partial double-stranded DNA donor and a single-strand portion that is complementary to the internal part of the sgRNA. An example donor sequence with such design is presented below. (FIG. 3C) The Internal-Anchor design with a closed partial double-stranded DNA donor and a single-strand portion that is complementary to the internal part of the sgRNA. An example donor sequence with such design is presented below. (FIG. 3D) The Internal-Anchor design with a closed partial single-stranded DNA donor and a single-strand portion that is complementary to the internal part of the sgRNA. An example donor sequence with such design is presented below. (FIG. 3E) The Internal-Anchor design with a hairpin (or hairpins) single-stranded DNA donor and a single-strand portion that is complementary to the internal part of the sgRNA. An example donor sequence with such design is presented below. The sequence in FIG. 3B is shown in SEQ ID NO: 6139, the sequence in FIG. 3C is shown in SEQ ID NO: 6140, the sequence in FIG. 3D is shown in SEQ ID NO: 6141, and the sequence in FIG. 3E is shown in SEQ ID NO: 6142.

FIG. 4 shows the effect of different positions of the fragments of donor DNA that is complementary to the guide RNA (referred to as “tail” hereafter) of the Internal-Anchor CRISPR system on the efficiencies of desired gene editing (in this non-limiting example, ‘CTT’ insertion at the target locus) in HEK293T cells and measured by next-generation sequencing. The CRISPR editing system consists of a guide RNA that is partially complementary to a donor DNA. The complementary sequence in the RNA guide is located at the internal part of the guide (the Internal-Anchor design). Different designs of the tails of donor DNA are: “0” indicates donors without tails were used; “R” indicates that the tail is at the 3′ end of the donor DNA; “RL” indicates that one DNA tail is at the 3′ end and a second tail is at the 5′ end of the donor DNA; “L” indicates that one DNA tail is at the 5′ end of the donor DNA; and “L10aL” indicates an “L” design as described above is followed by ten deoxyadenosines and a second “L.” The last sample used the guide and “L10aL” donor DNA, but without adding cas9 as a negative control.

FIG. 5 illustrates the effect of donor tail sequences (matching or non-matching with the internal anchor sequence of the guide RNA) of the Internal-Anchor CRISPR system, using the Left tail (5′ end), on the efficiencies of desired gene editing in HEK293T cells and measured by next-generation sequencing. Under the guide, “WT” indicates a wildtype guide RNA without an insertion of the internal anchor (IA). “IA” indicates a guide RNA with an insertion of an IA where the tail in donor DNA binds to. Different designs for the tails of donor DNAs (e.g., “L” and “L10aL”) are similarly labeled as in FIG. 4.

FIG. 6 shows the effects of Cas9-to-guide ratio (ranging from 1:0.6 to 1:10) and tail design (“L” and “L10aL” similarly labeled as in FIG. 4) on the efficiencies of desired (‘CTT’ insertion at the target locus) and undesired (other indels) gene editing using the Internal-Anchor CRISPR system in HEK293T cells and measured by next-generation sequencing.

FIG. 7 shows effect of the tail-tail inner sequence length (0, 5, 10, 15, 20, 25, and 30 deoxyadenosines) of the donor DNA on the efficiencies of desired (‘CTT’ insertion at the target locus) and undesired (other indels) gene editing using the Internal-Anchor CRISPR system in HEK293T cells and measured by next-generation sequencing.

FIG. 8 illustrates effects of homology arm lengths, of both distal (D) and proximal (P) relative to the “protospacer adjacent motif” (PAM) position, on the efficiencies of desired (‘CTT’ insertion at the target locus) and undesired (other indels) gene editing using the Internal-Anchor CRISPR system in HEK293T cells and measured by next-generation sequencing.

FIGS. 9A-9E show off-target effect and probabilities of translocation using some exemplary configurations with the “Internal Anchor design” (FIG. 9A) Genome-wide target profiling when targeting the HEK3 site using the Internal-Anchor CRISPR system with the different guides (WT and IA), donor DNA tails (0 and L10aL) and homology arms (D20P16 and D20P36), and SpCas9 (WT). (FIG. 9B) Genome-wide target profiling when targeting the HEK3 site using the Internal-Anchor CRISPR system with the different guides (WT and IA), donor DNA tails (0 and L10aL) and homology arms (D20P16 and D20P36) and SpCas9 (HiFi). (FIG. 9C) Genome-wide target profiling when targeting the HEK3 site using the Internal-Anchor CRISPR system with the different guides (WT and IA), donor DNA tails (0 and L10aL) and homology arms (D20P16 and D20P36) and SpCas9 (eCas9). (FIG. 9D) The number of off-target sites under different experimental condition groups. (FIG. 9E) The number of total GUIDE-seq reads under the experimental condition groups. The sequences in FIG. 9A are shown in SEQ ID NOs: 6143-6204, the sequences in FIG. 9B are shown in SEQ ID NOs: 6205-6240, and the sequences in FIG. 9C are shown in SEQ ID NOs: 6241-6268.

FIGS. 10A-10C illustrate a design for deleting an ˜1 kb fragment on HEK3. (Abbreviations: asODN, anchored single-strand DNA; legRNA, lead editing guide RNA).

FIGS. 11A-11C illustrate deletion results using different asODN-legRNA designs. FIG. 11A: PCR amplification products of different designs. The expected deletion product size is 288 bp (arrow) and WT product 175 bp (arrowhead). FIG. 11B: Band intensity ratio of 288 bp (deletion) to 175 bp (WT) of Design A1. FIG. 11C: IGV visualization of deletion product alignment on HEK3 locus. The sequences in FIG. 11C are shown in SEQ ID NOs: 6269-6270.

FIGS. 12A-12C illustrate deletion results using different asODN-legRNA designs. FIG. 12A: PCR amplification products of different designs. The expected deletion product size is 172 bp and WT product 1236 bp. FIG. 12B: Band intensity ratio of deletion to WT of Design A1, with triplicates. FIG. 12C: IGV visualization of deletion product alignment on HPRT1 locus. The sequences in FIG. 12C are shown in SEQ ID NO: 6271-6272.

FIGS. 13A-13C illustrate insertion of a 1734 bp-fragment at the GAPDH site.

### DETAILED DESCRIPTION

The present disclosure is based, in part, on the surprising finding that, when designing a donor DNA to be coupled to an sgRNA, the location of the effective coupling is not random. As shown in figures and examples below, the coupling between the donor DNA and the sgRNA is located at least 5 nucleotides away from both 3′ and 5′ ends of the modified sgRNA.

Provided herein are compositions of the systems as described herein. Various embodiments regarding non-covalent bonds, locations of the internal anchors on the modified sgRNAs, length and sequences of the internal anchors of the modified sgRNAs/binding segments of the donor DNAs, linkers between one or more binding segments on donor DNAs, SOI, other features of the donor DNAs, and Cas proteins are disclosed respectively. Furthermore, provided herein are methods of using the systems described herein.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The term “CRISPR/Cas,” as used herein, can refer to a ribonucleoprotein complex, e.g., a two part component ribonucleoprotein complex, with single guide RNA (sgRNA) and a CRISPR-associated (Cas) endonuclease. In some cases, CRISPR/Cas comprises more than two components. The term “CRISPR” can refer to the Clustered Regularly Interspaced Short Palindromic Repeats and the related system thereof. CRISPR can be used as an adaptive defense system that enables bacteria and archaea to detect and silence foreign nucleic acids (e.g., from viruses or plasmids). CRISPR can be adapted for use in a variety of cell types to allow for polynucleotide editing in a sequence-specific manner. In some cases, one or more elements of a CRISPR system can be derived from a type I, type II, type III, type IV, type V, or type VI CRISPR system. In the CRISPR type II system, the guide RNA can interact with Cas and direct the nuclease activity of the Cas enzyme to a target region. The target region can comprise a “protospacer” and a “protospacer adjacent motif” (PAM), and both domains can be used for a Cas enzyme mediated activity (e.g., cleavage). The protospacer can be referred to as a target site (or a genomic target site). The sgRNA can pair with (or hybridize) the opposite strand of the protospacer (binding site) to direct the Cas enzyme to the target region. The PAM site can refer to a short sequence recognized by the Cas enzyme and, in some cases, required for the Cas enzyme activity. The sequence and number of nucleotides for the PAM site can differ depending on the type of the Cas enzyme.

The term “Cas,” as used herein, generally refers to a wild type Cas protein, a fragment thereof, or a mutant or variant thereof. The term “Cas,” “enzyme Cas,” “enzyme CRISPR,” “protein CRISPR,” or “protein Cas” can be used interchangeably throughout the present disclosure.

A Cas protein can comprise a protein of or derived from a CRISPR/Cas a type I, type II, type III, or type IV, which has an RNA-guided polynucleotide-binding or nuclease activity. Examples of suitable Cas proteins include CasX, Cas3, Cas4, Cas5, Cas5e (or CasD), Cas6, Cas6e, Cas6f, Cas7, Cas8a1, Cas8a2, Cas8b, Cas8c, Cas9 (also known as Csn1 and Csx12), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Cszl, Csx15, Csf1, Csf2, Csf3, Csf4, Cu1966, homologues thereof, and modified versions thereof. In some cases, a Cas protein can comprise a protein of or derived from a CRISPR/Cas type V or type VI system, and modified versions thereof. In some cases, a Cas protein can be a catalytically dead or inactive Cas (dCas). In some cases, a Cas protein can have reduced or minimal nuclease activity (i.e., deactivated Cas, or dCas). In some cases, a Cas protein can be operatively coupled to one or more additional proteins, such as a nucleic acid polymerase. In an example, a Cas protein can be a dCas that is fused to a reverse transcriptase.

The term “single guide RNA” or “sgRNA,” as used herein, can refer to an RNA molecule (or a group of RNA molecules collectively) that can bind to a Cas protein and aid in targeting the Cas protein to a specific location within a target polynucleotide (e.g., a DNA). A single guide RNA comprises a CRISPR RNA (crRNA) segment and a trans-activating crRNA (tracrRNA) segment. The term “crRNA” or “crRNA segment,” as used herein, refers to an RNA molecule or portion thereof that includes a polynucleotide-targeting guide sequence, a stem sequence, and, optionally, a 5′-overhang sequence. The term “tracrRNA” or “tracrRNA segment,” refers to an RNA molecule or portion thereof that includes a protein-binding segment (e.g., the protein-binding segment can be capable of interacting with a CRISPR-associated protein, such as a Cas9).

The term “polynucleotide” or “nucleic acid,” as used interchangeably herein, can refer to a polymeric form of nucleotides (e.g., ribonucleotides or deoxyribonucleotides) of any length. Thus, this term includes single-, double-, or multi-stranded DNA or RNA, genomic DNA, complementary DNA (cDNA), guide RNA (gRNA), messenger RNA (mRNA), DNA-RNA hybrids, or a polymer comprising purine and pyrimidine bases or other natural, chemically or biochemically modified, non-natural, or derivatized nucleotide bases. The term “oligonucleotide,” as used herein, can refer to a polynucleotide of between about 5 and about 100 nucleotides of single- or double-stranded DNA or RNA. The length of a nucleic acid can be referred to in reference of the number of bases in the nucleic acid sequence. For example, a sequence of 100 nucleotides can be referred to as being 100 bases in length. However, for the purposes of this disclosure, there can be no upper limit to the length of an oligonucleotide. In some cases, oligonucleotides can be known as “oligomers” or “oligos” and can be isolated from genes, or chemically synthesized by methods known in the art. The terms “polynucleotide” and “nucleic acid” can include single-stranded (such as sense or antisense) and double-stranded polynucleotides. Examples of nucleotides for DNA can include cytosine (C), guanine (G), adenine (A), thymine (T), or modifications thereof. Examples of nucleotides for RNA can include C, G, A, uracil (U), or modifications thereof.

A “subject” disclosed herein includes any living organism. Thus, in some embodiments, subjects are mammals, avians, reptiles, amphibians, fish, plants, fungi, or bacteria. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets. In some embodiments, suitable subjects include both males and females and subjects of any age, including embryonic (e.g., in-utero or in-ovo), infant, juvenile, adolescent, adult and geriatric subjects. In some embodiments, a subject is a human.

“Treating” or “treatment” can refer to both therapeutic treatment and prophylactic or preventative measures, wherein the object is to prevent or slow down (lessen) a targeted pathologic condition or disorder. Those in need of treatment can include those already with the disorder, as well as those prone to have the disorder, or those in whom the disorder is to be prevented. For example, a subject can be successfully “treated” for a disease caused by a gain-of-function mutation, if, after receiving a therapeutic amount of a composition according to the methods of the present disclosure, the subject shows observable and/or measurable reduction in or absence of one or more of the following: relief to some extent of one or more of the symptoms associated with the specific disease; reduced morbidity and/or mortality, and improvement in quality of life issues.

Certain ranges are presented herein with numerical values being preceded by the term “about.” The term “about” can be used herein to provide literal support for the exact number that it precedes, as well as a number that is near to or approximately the number that the term precedes. In determining whether a number is near to or approximately a specifically recited number, the near or approximating un-recited number can be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, can be encompassed within the methods and compositions described herein are. The upper and lower limits of these smaller ranges can independently be included in the smaller ranges and are also encompassed within the methods and compositions described herein, subject to any specifically excluded limit in the stated range. Where the stated range includes one or both of the limits, ranges excluding either or both of those included limits can also be included in the methods and compositions described herein.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “desired editing efficiency” or “percentage of desired editing” used herein is referred to an expected editing outcome with the designed sequence and designed location, based on the design of SOI in the donor DNAs described herein. In some embodiments where the SOI comprises the target sequence with one or more nucleotide substitution, one or more nucleotide insertion, one or more nucleotide deletion, or any combination thereof, the desired editing efficiency or the percentage of the desired editing is defined as the proportion of the corresponding gene editing products comprising the target sequence with one or more nucleotide substitution, one or more nucleotide insertion, one or more nucleotide deletion, or any combination thereof, and at the expected loci based on the design of the upstream and/or downstream homology arms if the second portion of the donor DNA has any homology arms.

The term “upstream homology arm” used herein is referred to a segment of target sequence or a region that is between the first and second loci that is at the 5′ side of the cutting site. The term “downstream homology arm” used herein is referred to a segment of target sequence or a region that is between the first and second loci that is at the 3′ side of the cutting site. The term “distal/proximal homology arm” used herein is referred to a segment of target sequence or a region that is between the first and second loci that is distal or proximal relative to protospacer adjacent motif (PAM) site.

### 1. Composition of the Systems as Described Herein

In one aspect, provided herein is a system for altering a target sequence, comprising a modified single-guide RNA (sgRNA) and a donor DNA, wherein the modified sgRNA comprises a CRISPR RNA (crRNA) and a trans-active RNA (tracrRNA), wherein the modified sgRNA comprises one or more internal anchors that are at least 5 nucleotides away from both 3′ and 5′ ends of the modified sgRNA, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI). In another aspect, provided here in a system comprising a donor DNA and two modified single-guide RNAs (sgRNAs) for cutting at a first locus on a first chromosome and a second locus on a second chromosome, wherein each of the modified sgRNAs comprises a CRISPR RNA (crRNA) and a trans-active RNA (tracrRNA), wherein each of the modified sgRNAs comprises one or more internal anchors that are at least 5 nucleotides away from both 3′ and 5′ ends of each of the modified sgRNAs, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI), wherein the donor DNA comprises an upstream homology arm and/or a downstream homology arm.

### (a) Non-Covalent Bonds

In some embodiments, the non-covalent bond described herein is a hydrogen bond. In specific embodiments, the non-covalent bond described herein is a Watson-Crick interaction. In other embodiments, the non-covalent bond described herein is an ionic interaction. In other embodiments, the non-covalent bond described herein is a Van der Waals interaction. In other embodiments, the non-covalent bond described herein is a hydrophobic bond.

(b) Locations of the Internal Anchors on the Modified sgRNAs

In some embodiments, the modified sgRNA comprises a nexus, a first hairpin, and a single-stranded region between the tracrRNA and the crRNA. Accordingly, in some specific embodiment, an internal anchor of the one or more internal anchors is located in a single-stranded region between the tracrRNA and the crRNA. In other specific embodiments, an internal anchor of the one or more internal anchors is located in a single-stranded region within the first hairpin. In other specific embodiments, an internal anchor of the one or more internal anchors is located in a single-stranded region between the nexus and the first hairpin. In other specific embodiments, an internal anchor of the one or more internal anchors is located in the stem portion of the first hairpin, thus resulting an artificial bulge-like structure in the stem portion of the first hairpin. In other specific embodiments, because one or more sequences in the tracrRNA are reverse complementary to counterpart sequences in crRNA, stems are formed between the tracrRNA and the crRNA. Accordingly, an internal anchor of the one or more internal anchors is located in the stem between the tracrRNA and the crRNA, thus resulting an artificial bulge-like structure in the stem.

In some embodiments, the modified sgRNA comprises a nexus, a first hairpin, a second hairpin, an optionally one or more hairpins, and a single-stranded region between the tracrRNA and the crRNA. Accordingly, in some specific embodiment, an internal anchor of the one or more internal anchors is located in a single-stranded region between the tracrRNA and the crRNA. In other specific embodiments, an internal anchor of the one or more internal anchors is located in a single-stranded region within the first hairpin. In other specific embodiments, an internal anchor of the one or more internal anchors is located in a single-stranded region within the second hairpin. In other specific embodiments, an internal anchor of the one or more internal anchors is located in a single-stranded region within the optionally one or more hairpins. In other specific embodiments, an internal anchor of the one or more internal anchors is located in a single-stranded region between the nexus and the first hairpin. In other specific embodiments, an internal anchor of the one or more internal anchors is located in a single-stranded region between the first hairpin and the second hairpin. In other specific embodiments, an internal anchor of the one or more internal anchors is located in a single-stranded region between the second hairpin and the third hairpins, or the two neighboring hairpins. In other specific embodiments, an internal anchor of the one or more internal anchors is located in the stem portion of the first hairpin, thus resulting an artificial bulge-like structure in the stem portion of the first hairpin. In other specific embodiments, an internal anchor of the one or more internal anchors is located in the stem portion of the second hairpin, thus resulting an artificial bulge-like structure in the stem portion of the second hairpin. In other specific embodiments, an internal anchor of the one or more internal anchors is located in the stem portion of the optionally one or more hairpins, thus resulting an artificial bulge-like structure in the stem portion of the optionally one or more hairpins. In other specific embodiments, because one or more sequences in the tracrRNA are reverse complementary to counterpart sequences in crRNA, stems are formed between the tracrRNA and the crRNA. Accordingly, an internal anchor of the one or more internal anchors is located in the stem between the tracrRNA and the crRNA, thus resulting an artificial bulge-like structure in the stem.

In some embodiments, the modified sgRNA comprises a nexus, a bulge region, a first hairpin, and a single-stranded region between the tracrRNA and the crRNA. Specifically, because one or more sequences in the tracrRNA are reverse complementary to counterpart sequences in crRNA, stems are formed between the tracrRNA and the crRNA. The stems are split by the bulge region into an upper stem and a lower stem. Accordingly, in some specific embodiment, an internal anchor of the one or more internal anchors is located in a single-stranded region between the tracrRNA and the crRNA, which corresponds to the loop on top of the upper stem. In other specific embodiments, an internal anchor of the one or more internal anchors is located in a single-stranded region within the first hairpin. In other specific embodiments, an internal anchor of the one or more internal anchors is located in a single-stranded region between the nexus and the first hairpin. In other specific embodiments, an internal anchor of the one or more internal anchors is located in the stem portion of the first hairpin, thus resulting an artificial bulge-like structure in the stem portion of the first hairpin. In other specific embodiments, an internal anchor of the one or more internal anchors is located within the upper stem, thus resulting an artificial bulge-like structure in the upper stem. In other specific embodiments, an internal anchor of the one or more internal anchors is located within the lower stem, thus resulting an artificial bulge-like structure in the lower stem.

In some embodiments, the modified sgRNA comprises a nexus, a bulge region, a first hairpin, a second hairpin, optionally one or more hairpins, and a single-stranded region between the tracrRNA and the crRNA. Specifically, because one or more sequences in the tracrRNA are reverse complementary to counterpart sequences in crRNA, stems are formed between the tracrRNA and the crRNA. The stems are split by the bulge region into an upper stem and a lower stem. Accordingly, in some specific embodiment, an internal anchor of the one or more internal anchors is located in a single-stranded region between the tracrRNA and the crRNA, which corresponds to the loop on top of the upper stem. In other specific embodiments, an internal anchor of the one or more internal anchors is located in a single-stranded region within the first hairpin. In other specific embodiments, an internal anchor of the one or more internal anchors is located in a single-stranded region within the second hairpin. In other specific embodiments, an internal anchor of the one or more internal anchors is located in a single-stranded region within the optionally one or more hairpins. In other specific embodiments, an internal anchor of the one or more internal anchors is located in a single-stranded region between the nexus and the first hairpin. In other specific embodiments, an internal anchor of the one or more internal anchors is located in a single-stranded region between the first hairpin and the second hairpin, or any neighboring hairpins. In other specific embodiments, an internal anchor of the one or more internal anchors is located in the stem portion of the first hairpin, thus resulting an artificial bulge-like structure in the stem portion of the first hairpin. In other specific embodiments, an internal anchor of the one or more internal anchors is located in the stem portion of the second hairpin, thus resulting an artificial bulge-like structure in the stem portion of the second hairpin. In other specific embodiments, an internal anchor of the one or more internal anchors is located in the stem portion of the optionally one or more hairpins, thus resulting an artificial bulge-like structure in the stem portion of the one or more hairpins. In other specific embodiments, an internal anchor of the one or more internal anchors is located within the upper stem, thus resulting an artificial bulge-like structure in the upper stem. In other specific embodiments, an internal anchor of the one or more internal anchors is located within the lower stem, thus resulting an artificial bulge-like structure in the lower stem.

(c) Length of the Internal Anchors of the Modified sgRNAs/Binding Segments of the Donor DNAs

In some embodiments, each of the one or more internal anchors is 3-nucleotide to 100-nucleotide long. In other embodiments, each of the one or more internal anchors is 3-nucleotide to 20-nucleotide long. In other embodiments, each of the one or more internal anchors is at least 3-nucleotide long. In other embodiments, each of the one or more internal anchors is at least 4-nucleotide long. In other embodiments, each of the one or more internal anchors is at least 5-nucleotide long. In other embodiments, each of the one or more internal anchors is at least 6-nucleotide long. In other embodiments, each of the one or more internal anchors is at least 7-nucleotide long. In other embodiments, each of the one or more internal anchors is at least 8-nucleotide long. In other embodiments, each of the one or more internal anchors is at least 9-nucleotide long. In other embodiments, each of the one or more internal anchors is at least 10-nucleotide long. In other embodiments, each of the one or more internal anchors is 3-nucleotide long. In other embodiments, each of the one or more internal anchors is 4-nucleotide long. In other embodiments, each of the one or more internal anchors is 5-nucleotide long. In other embodiments, each of the one or more internal anchors is 6-nucleotide long. In other embodiments, each of the one or more internal anchors is 7-nucleotide long. In other embodiments, each of the one or more internal anchors is 8-nucleotide long. In other embodiments, each of the one or more internal anchors is 9-nucleotide long. In other embodiments, each of the one or more internal anchors is 10-nucleotide long.

In some embodiments, the binding segments of the donor DNAs bind to the internal anchors of the modified sgRNAs via a Watson-Crick interaction. Therefore, the binding segments of the donor DNAs also share similar length as the internal anchors of the modified sgRNAs. Accordingly, in some embodiments, each of the one or more binding segments is 3-nucleotide to 100-nucleotide long. In other embodiments, each of the one or more binding segments is 3-nucleotide to 20-nucleotide long. In other embodiments, each of the one or more binding segments is at least 3-nucleotide long. In other embodiments, each of the one or more binding segments is at least 4-nucleotide long. In other embodiments, each of the one or more binding segments is at least 5-nucleotide long. In other embodiments, each of the one or more binding segments is at least 6-nucleotide long. In other embodiments, each of the one or more binding segments is at least 7-nucleotide long. In other embodiments, each of the one or more binding segments is at least 8-nucleotide long. In other embodiments, each of the one or more binding segments is at least 9-nucleotide long. In other embodiments, each of the one or more binding segments is at least 10-nucleotide long. In other embodiments, each of the one or more binding segments is 3-nucleotide long. In other embodiments, each of the one or more binding segments is 4-nucleotide long. In other embodiments, each of the one or more binding segments is 5-nucleotide long. In other embodiments, each of the one or more binding segments is 6-nucleotide long. In other embodiments, each of the one or more binding segments is 7-nucleotide long. In other embodiments, each of the one or more binding segments is 8-nucleotide long. In other embodiments, each of the one or more binding segments is 9-nucleotide long. In other embodiments, each of the one or more binding segments is 10-nucleotide long.

(d) Sequence of the Internal Anchors of the Modified sgRNAs/Binding Segments of the Donor DNAs

In some embodiments, each of the internal anchors of the modified sgRNAs comprises a sequence that is an uncommon motif in a host genome. In specific embodiments, in some embodiments, each of the internal anchors of the modified sgRNAs comprises a sequence that is an uncommon motif in human genome. As non-limiting examples, in some embodiments, each of the internal anchors of the modified sgRNAs comprises a sequence from Table 1. In another embodiments, each of the internal anchors of the modified sgRNAs comprises a sequence from Table 2. In some embodiments, the binding segments of the donor DNAs bind to the internal anchors of the modified sgRNAs via a Watson-Crick interaction. Therefore, in some embodiments, each of the binding segments of the donor DNAs comprises a sequence that is reverse complementary to a sequence from Table 1. In other embodiments, each of the binding segments of the donor DNAs comprises a sequence that is reverse complementary to a sequence from Table 2.

In some embodiments, each of the binding segments of the donor DNAs comprises a sequence that is an uncommon motif in a host genome. In specific embodiments, in some embodiments, each of the binding segments of the donor DNAs comprises a sequence that is an uncommon motif in human genome. As non-limiting examples, in some embodiments, each of the binding segments of the donor DNAs comprises a sequence from Table 3. In another embodiments, each of the binding segments of the donor DNAs comprises a sequence from Table 4. In some embodiments, the binding segments of the donor DNAs bind to the internal anchors of the modified sgRNAs via a Watson-Crick interaction. Therefore, in some embodiments, each of the internal anchors of the modified sgRNAs comprises a sequence that is reverse complementary to a sequence from Table 3. In some embodiments, each of the internal anchors of the modified sgRNAs comprises a sequence that is reverse complementary to a sequence from Table 4.

TABLE 1
Exemplary sequences of internal anchors of
the modified sgRNAs (5-nucleotide long)

| SEQ ID | |
|---|---|
| NO. | Sequence |
| 1 | CCCAG |
| 2 | UCCCA |
| 3 | CAGAG |
| 4 | CCAGG |
| 5 | AGGAG |
| 6 | CAGGA |
| 7 | CCUCC |
| 8 | CUCAG |
| 9 | CCAGC |
| 10 | AGGCA |
| 11 | CUGCA |
| 12 | CAGCC |
| 13 | CACAG |
| 14 | CAGCA |
| 15 | CACUG |
| 16 | CUCCA |
| 17 | CUCCC |
| 18 | CAGGC |
| 19 | AGCCU |
| 20 | CACAC |
| 21 | CCUCA |
| 22 | AGCCA |
| 23 | CUGGA |
| 24 | GGAGA |
| 25 | CUUCC |
| 26 | AGCUG |
| 27 | CACCA |
| 28 | GAGGC |
| 29 | AGAGG |
| 30 | AGCAG |
| 31 | CUCUC |
| 32 | CUGCC |
| 33 | ACAGG |
| 34 | AGGGA |
| 35 | CCUGA |
| 36 | CCACA |
| 37 | CCACC |
| 38 | GAGGA |
| 39 | GGGAA |
| 40 | GCAGA |
| 41 | CCAGA |
| 42 | AGGUG |
| 43 | CUCAC |
| 44 | UGCCA |
| 45 | CAGGG |
| 46 | CCUUC |
| 47 | AGUGG |
| 48 | CCCAA |
| 49 | GCUGA |
| 50 | CAUGG |
| 51 | CCUGC |
| 52 | ACCUG |
| 53 | CUGGC |
| 54 | GAGAC |
| 55 | CUGUC |
| 56 | CCAAG |
| 57 | GCUCA |
| 58 | ACCCA |
| 59 | ACUGC |
| 60 | ACCUC |
| 61 | GGCCA |
| 62 | CCCAC |
| 63 | CCAUC |
| 64 | CAAGG |
| 65 | GCCCA |
| 66 | ACUCC |
| 67 | AAGGG |
| 68 | GGUGA |
| 69 | CACUC |
| 70 | AGAGC |
| 71 | GUGGA |
| 72 | AUGGG |
| 73 | GCACA |
| 74 | CCCUC |
| 75 | ACCAG |
| 76 | ACCAC |
| 77 | GGGCA |
| 78 | GCCAC |
| 79 | AGGCC |
| 80 | GAGCA |
| 81 | ACUGG |
| 82 | CAUGC |
| 83 | ACAGC |
| 84 | GUGCA |
| 85 | GCCAA |
| 86 | CACCC |
| 87 | AGCAC |
| 88 | AUCCC |
| 89 | AAGGC |
| 90 | AGCUC |
| 91 | GGCAA |
| 92 | GAGCC |
| 93 | AGUGC |
| 94 | AUGCC |
| 95 | GCAGC |
| 96 | GAAGC |
| 97 | AUGGC |
| 98 | AAGCC |
| 99 | CAUCC |
| 100 | GGACA |
| 101 | CAGAC |
| 102 | CUUGC |
| 103 | CUGAC |
| 104 | AACCC |
| 105 | AGACC |
| 106 | AGCCC |
| 107 | ACCCU |
| 108 | ACACC |
| 109 | CAAGC |
| 110 | CCAAC |
| 111 | GACCA |
| 112 | CAGUC |
| 113 | AGGGC |
| 114 | AGGAC |
| 115 | GGUCA |
| 116 | GCUCC |
| 117 | AGGUC |
| 118 | GGCAC |
| 119 | CAACC |
| 120 | AGUCC |
| 121 | GACAC |
| 122 | CCUAG |
| 123 | GGAAC |
| 124 | GUCCA |
| 125 | GUCAC |
| 126 | GCAAC |
| 127 | GACUC |
| 128 | GAACC |
| 129 | GGGAC |
| 130 | GCACC |
| 131 | GGCUA |
| 132 | GAUGC |
| 133 | CCCUA |
| 134 | CUACC |
| 135 | GAUCC |
| 136 | GCUAC |
| 137 | GCCUA |
| 138 | CCUAC |
| 139 | GACCC |
| 140 | GGGUA |
| 141 | CUAGC |
| 142 | GGACC |
| 143 | GGUAC |
| 144 | AGGCG |
| 145 | CCGAG |
| 146 | CACGC |
| 147 | CGAGA |
| 148 | CGGGA |
| 149 | CGUGA |
| 150 | CCACG |
| 151 | ACGUG |
| 152 | CCUCG |
| 153 | AGACG |
| 154 | CGCCA |
| 155 | CGGAG |
| 156 | AGCCG |
| 157 | ACGCC |
| 158 | CACGG |
| 159 | CACCG |
| 160 | ACACG |
| 161 | ACGGG |
| 162 | GCCGA |
| 163 | AGCGA |
| 164 | CAGCG |
| 165 | CCGUC |
| 166 | CGUGC |
| 167 | ACCCG |
| 168 | CGCAG |
| 169 | AAGCG |
| 170 | CACGA |
| 171 | CCCGA |
| 172 | CUCGA |
| 173 | ACGGA |
| 174 | GCGGA |
| 175 | CGCUC |
| 176 | ACGAG |
| 177 | CCGCA |
| 178 | CUCGC |
| 179 | AUCGC |
| 180 | ACGCA |
| 181 | ACUCG |
| 182 | CGAAG |
| 183 | GGCGA |
| 184 | ACCGU |
| 185 | AGCGG |
| 186 | AACGG |
| 187 | CGGCA |
| 188 | CAACG |
| 189 | CGGAA |
| 190 | CGCAC |
| 191 | ACGCU |
| 192 | AGCGC |
| 193 | ACCGC |
| 194 | GCGCA |
| 195 | CGAUC |
| 196 | CGUUC |
| 197 | CGUCA |
| 198 | CCGGA |
| 199 | CGACA |
| 200 | CAUCG |
| 201 | CGUCC |
| 202 | ACGUC |
| 203 | CCGAA |
| 204 | AUCCG |
| 205 | ACGGC |
| 206 | AUGCG |
| 207 | UCGCA |
| 208 | CGAGC |
| 209 | CGCAA |
| 210 | AACGC |
| 211 | AACCG |
| 212 | CGAAC |
| 213 | GACGA |
| 214 | ACCGA |
| 215 | UCCGA |
| 216 | CGUAG |
| 217 | GCGAA |
| 218 | ACCGG |
| 219 | GACGC |
| 220 | AGUCG |
| 221 | CCGUA |
| 222 | CCGAC |
| 223 | GCGAC |
| 224 | CGGUC |
| 225 | CGACC |
| 226 | CGGAC |
| 227 | ACGAC |
| 228 | AUCGG |
| 229 | CGGUA |
| 230 | CGCUA |
| 231 | GCGUA |
| 232 | GUCGA |
| 233 | CGUAC |
| 234 | CGCGA |
| 235 | ACGCG |
| 236 | CGACG |
| 237 | CCCAG |
| 238 | UCCCA |
| 239 | CAGAG |
| 240 | CCAGG |
| 241 | AGGAG |
| 242 | CAGGA |
| 243 | CCUCC |
| 244 | CUCAG |
| 245 | CCAGC |
| 246 | AGGCA |
| 247 | CUGCA |
| 248 | CAGCC |
| 249 | CACAG |
| 250 | CAGCA |
| 251 | CACUG |
| 252 | CUCCA |
| 253 | CUCCC |
| 254 | CAGGC |
| 255 | AGCCU |
| 256 | CACAC |
| 257 | CCUCA |
| 258 | AGCCA |
| 259 | CUGGA |
| 260 | GGAGA |
| 261 | CUUCC |
| 262 | AGCUG |
| 263 | CACCA |
| 264 | GAGGC |
| 265 | AGAGG |
| 266 | AGCAG |
| 267 | CUCUC |
| 268 | CUGCC |
| 269 | ACAGG |
| 270 | AGGGA |
| 271 | CCUGA |
| 272 | CCACA |
| 273 | CCACC |
| 274 | GAGGA |
| 275 | GGGAA |
| 276 | GCAGA |
| 277 | CCAGA |
| 278 | AGGUG |
| 279 | CUCAC |
| 280 | UGCCA |
| 281 | CAGGG |
| 282 | CCUUC |
| 283 | AGUGG |
| 284 | CCCAA |
| 285 | GCUGA |
| 286 | CAUGG |
| 287 | CCUGC |
| 288 | ACCUG |
| 289 | CUGGC |
| 290 | GAGAC |
| 291 | CUGUC |
| 292 | CCAAG |
| 293 | GCUCA |
| 294 | ACCCA |
| 295 | ACUGC |
| 296 | ACCUC |
| 297 | GGCCA |
| 298 | CCCAC |
| 299 | CCAUC |
| 300 | CAAGG |
| 301 | GCCCA |
| 302 | ACUCC |
| 303 | AAGGG |
| 304 | GGUGA |
| 305 | CACUC |
| 306 | AGAGC |
| 307 | GUGGA |
| 308 | AUGGG |
| 309 | GCACA |
| 310 | CCCUC |
| 311 | ACCAG |
| 312 | ACCAC |
| 313 | GGGCA |
| 314 | GCCAC |
| 315 | AGGCC |
| 316 | GAGCA |
| 317 | ACUGG |
| 318 | CAUGC |
| 319 | ACAGC |
| 320 | GUGCA |
| 321 | GCCAA |
| 322 | CACCC |
| 323 | AGCAC |
| 324 | AUCCC |
| 325 | AAGGC |
| 326 | AGCUC |
| 327 | GGCAA |
| 328 | GAGCC |
| 329 | AGUGC |
| 330 | AUGCC |
| 33 | GCAGC |
| 332 | GAAGC |
| 333 | AUGGC |
| 334 | AAGCC |
| 335 | CAUCC |
| 336 | GGACA |
| 337 | CAGAC |
| 338 | CUUGC |
| 339 | CUGAC |
| 340 | AACCC |
| 341 | AGACC |
| 342 | AGCCC |
| 343 | ACCCU |
| 344 | ACACC |
| 345 | CAAGC |
| 346 | CCAAC |
| 347 | GACCA |
| 348 | CAGUC |
| 349 | AGGGC |
| 350 | AGGAC |
| 351 | GGUCA |
| 352 | GCUCC |
| 353 | AGGUC |
| 354 | GGCAC |
| 355 | CAACC |
| 356 | AGUCC |
| 357 | GACAC |
| 358 | CCUAG |
| 359 | GGAAC |
| 360 | GUCCA |
| 361 | GUCAC |
| 362 | GCAAC |
| 363 | GACUC |
| 364 | GAACC |
| 365 | GGGAC |
| 366 | GCACC |
| 367 | GGCUA |
| 368 | GAUGC |
| 369 | CCCUA |
| 370 | CUACC |
| 371 | GAUCC |
| 372 | GCUAC |
| 373 | GCCUA |
| 374 | CCUAC |
| 375 | GACCC |
| 376 | GGGUA |
| 377 | CUAGC |
| 378 | GGACC |
| 379 | GGUAC |
| 380 | AGGCG |
| 381 | CCGAG |
| 382 | CACGC |
| 383 | CGAGA |
| 384 | CGGGA |
| 385 | CGUGA |
| 386 | CCACG |
| 387 | ACGUG |
| 388 | CCUCG |
| 389 | AGACG |
| 390 | CGCCA |
| 391 | CGGAG |
| 392 | AGCCG |
| 393 | ACGCC |
| 394 | CACGG |
| 395 | CACCG |
| 396 | ACACG |
| 397 | ACGGG |
| 398 | GCCGA |
| 399 | AGCGA |
| 400 | CAGCG |
| 401 | CCGUC |
| 402 | CGUGC |
| 403 | ACCCG |
| 404 | CGCAG |
| 405 | AAGCG |
| 406 | CACGA |
| 407 | CCCGA |
| 408 | CUCGA |
| 409 | ACGGA |
| 410 | GCGGA |
| 411 | CGCUC |
| 412 | ACGAG |
| 413 | CCGCA |
| 414 | CUCGC |
| 415 | AUCGC |
| 416 | ACGCA |
| 417 | ACUCG |
| 418 | CGAAG |
| 419 | GGCGA |
| 420 | ACCGU |
| 421 | AGCGG |
| 422 | AACGG |
| 423 | CGGCA |
| 424 | CAACG |
| 425 | CGGAA |
| 426 | CGCAC |
| 427 | ACGCU |
| 428 | AGCGC |
| 429 | ACCGC |
| 430 | GCGCA |
| 431 | CGAUC |
| 432 | CGUUC |
| 433 | CGUCA |
| 434 | CCGGA |
| 435 | CGACA |
| 436 | CAUCG |
| 437 | CGUCC |
| 438 | ACGUC |
| 439 | CCGAA |
| 440 | AUCCG |
| 441 | ACGGC |
| 442 | AUGCG |
| 443 | UCGCA |
| 444 | CGAGC |
| 445 | CGCAA |
| 446 | AACGC |
| 447 | AACCG |
| 448 | CGAAC |
| 449 | GACGA |
| 450 | ACCGA |
| 451 | UCCGA |
| 452 | CGUAG |
| 453 | GCGAA |
| 454 | ACCGG |
| 455 | GACGC |
| 456 | AGUCG |
| 457 | CCGUA |
| 458 | CCGAC |
| 459 | GCGAC |
| 460 | CGGUC |
| 461 | CGACC |
| 462 | CGGAC |
| 463 | ACGAC |
| 464 | AUCGG |
| 465 | CGGUA |
| 466 | CGCUA |
| 467 | GCGUA |
| 468 | GUCGA |
| 469 | CGUAC |
| 470 | CGCGA |
| 47 | ACGCG |
| 472 | CGACG |

TABLE 2
Exemplary sequences of internal anchors
of the modified sgRNAs (6-nucleotide long)

| SEQ ID NO. | Sequence |
|---|---|
| 473 | AGGCUG |
| 474 | CACACA |
| 475 | CCUCCC |
| 476 | CAGGAG |
| 477 | CUGGGA |
| 478 | CCCAGC |
| 479 | ACACAC |
| 480 | CUCCCA |
| 481 | CCCAGG |
| 482 | AGGCAG |
| 483 | ACAGAG |
| 484 | AGAGAG |
| 485 | CCAGGC |
| 486 | GCCUCC |
| 487 | CCAGCC |
| 488 | AGGAAG |
| 489 | AGGAGA |
| 490 | CCUCAG |
| 491 | GAGAGA |
| 492 | AGCCUG |
| 493 | GGAGAA |
| 494 | CAGAGA |
| 495 | ACUGCA |
| 496 | CUCAGC |
| 497 | AGACAG |
| 498 | CACUGC |
| 499 | AGGAGG |
| 500 | CCCAAA |
| 501 | CUGAGA |
| 502 | CUCCAG |
| 503 | CAGGCA |
| 504 | AGCUGG |
| 505 | AGUGAG |
| 506 | CACAGA |
| 507 | UCAGGA |
| 508 | CAGGAA |
| 509 | CCACCA |
| 510 | CCUGCC |
| 511 | CAGUGA |
| 512 | CUGGGC |
| 513 | CCUGGC |
| 514 | UCCCAA |
| 515 | CCAGGA |
| 516 | CAGAAG |
| 517 | CCAAAG |
| 518 | CCAGCA |
| 519 | GAGGCA |
| 520 | AUCCCA |
| 521 | AGCCUC |
| 522 | CAGUGG |
| 523 | GGCUCA |
| 524 | GACAGA |
| 525 | UGGGAA |
| 526 | GGAAGA |
| 527 | AGGGAA |
| 528 | GGGAAA |
| 529 | CUGGAA |
| 530 | UCUCCA |
| 531 | GAGGAA |
| 532 | CACCUG |
| 533 | GAAGGA |
| 534 | ACACAG |
| 535 | GGCUGA |
| 536 | GCCUCA |
| 537 | AGAGGA |
| 538 | GAGACA |
| 539 | GCUGGA |
| 540 | CUUUCC |
| 541 | CUGCAA |
| 542 | CUUCUC |
| 543 | CCUGAG |
| 544 | CCUGUA |
| 545 | CAGAGG |
| 573 | AAGCAG |
| 574 | CUUCCC |
| 575 | CCUUUC |
| 576 | AAUCCC |
| 577 | AGAGCA |
| 578 | GCAGGA |
| 579 | GCAGAA |
| 580 | UCCUCA |
| 581 | CCCACC |
| 582 | CUCUUC |
| 583 | CCUCCA |
| 584 | CAGCCA |
| 585 | AAGGGA |
| 586 | CUCACA |
| 587 | AAGGCA |
| 588 | AGAGAC |
| 589 | CAGGGA |
| 590 | CAGCAC |
| 591 | ACAGCA |
| 592 | ACAUGG |
| 593 | CUGGCC |
| 594 | UCACCA |
| 595 | ACAGUG |
| 596 | AAGAGG |
| 597 | CACCCA |
| 598 | CUCUGA |
| 599 | GGGAGA |
| 600 | AGACCA |
| 601 | AGCCAC |
| 602 | AGCACU |
| 603 | ACCACA |
| 604 | CACAUG |
| 605 | CUCCUC |
| 606 | CAGAGC |
| 607 | CAGCAA |
| 608 | AAGCCA |
| 609 | CCCAGA |
| 610 | CCACAG |
| 611 | CUGAAG |
| 612 | CAGCUA |
| 613 | GCUCAC |
| 614 | CUCUCC |
| 615 | AGGCAU |
| 616 | ACCUCA |
| 617 | CCCUCC |
| 618 | CCUGGA |
| 619 | ACCAUG |
| 620 | CAGACA |
| 621 | CCUCUC |
| 622 | UCCAGA |
| 623 | AGCUGA |
| 624 | CAAAGG |
| 625 | AUGGAG |
| 626 | AGAGUG |
| 627 | GUGAGA |
| 628 | AGGCCA |
| 629 | CUGCAC |
| 630 | ACUCCU |
| 631 | AAACCC |
| 632 | AAAGGG |
| 633 | UCAGCA |
| 634 | CCAGAG |
| 635 | CCAACA |
| 636 | AGCCAA |
| 637 | GAGGGA |
| 638 | GAAGCA |
| 639 | CAGAUG |
| 640 | GAGGCC |
| 641 | ACUGUG |
| 642 | CAUGCC |
| 643 | CCUGAA |
| 644 | CCAAGA |
| 645 | CUCUCA |
| 646 | UGCACA |
| 647 | AUGCUG |
| 648 | CCUCAA |
| 649 | AACUCC |
| 650 | CUGCCA |
| 651 | CACUCC |
| 652 | AGGUGA |
| 653 | CACAGG |
| 654 | AGCAAG |
| 655 | AAGCUG |
| 656 | ACAGGA |
| 657 | CACCUC |
| 658 | AGCAGG |
| 659 | AGGUCA |
| 660 | GCCACC |
| 661 | AGUGCA |
| 662 | AGAAGC |
| 663 | AGGCAA |
| 664 | ACACUG |
| 665 | CCAUUC |
| 666 | CACAGC |
| 667 | CUGCCC |
| 668 | CUCAAG |
| 669 | ACUCAG |
| 670 | CUGCUC |
| 671 | UCCACA |
| 672 | AUGGGA |
| 645 | CUCUCA |
| 646 | UGCACA |
| 647 | AUGCUG |
| 648 | CCUCAA |
| 649 | AACUCC |
| 650 | CUGCCA |
| 651 | CACUCC |
| 652 | AGGUGA |
| 653 | CACAGG |
| 654 | AGCAAG |
| 655 | AAGCUG |
| 656 | ACAGGA |
| 657 | CACCUC |
| 658 | AGCAGG |
| 659 | AGGUCA |
| 660 | GCCACC |
| 661 | AGUGCA |
| 662 | AGAAGC |
| 663 | AGGCAA |
| 664 | ACACUG |
| 665 | CCAUUC |
| 666 | CACAGC |
| 667 | CUGCCC |
| 668 | CUCAAG |
| 669 | ACUCAG |
| 670 | CUGCUC |
| 671 | UCCACA |
| 672 | AUGGGA |
| 673 | CAAGUG |
| 674 | GGAAAC |
| 675 | AGGACA |
| 676 | CAGCUC |
| 677 | CAAGGA |
| 678 | GAAACC |
| 679 | AUGGCA |
| 680 | CCUUCA |
| 681 | AGCUCA |
| 682 | CUGUCA |
| 683 | CAUGAG |
| 684 | CACACC |
| 685 | CUCCAA |
| 686 | GACACA |
| 687 | ACCUGG |
| 688 | CACUGA |
| 689 | AAGUGG |
| 690 | CAAGAG |
| 691 | AUGUGG |
| 692 | AGGAUG |
| 693 | ACCUCC |
| 694 | GAUGGA |
| 695 | AGAGGG |
| 696 | CCACCC |
| 697 | CCAGGG |
| 698 | CAUGGA |
| 699 | ACCUCU |
| 700 | GUGGGA |
| 701 | GUGGCA |
| 702 | CAUUCC |
| 703 | ACUGAG |
| 704 | AGUGGC |
| 705 | AGCACA |
| 706 | CCAUCA |
| 707 | AUGCAG |
| 708 | AAGUGC |
| 709 | CAAAGC |
| 710 | CUUUGC |
| 711 | GGUGGA |
| 712 | GUGGAA |
| 713 | AGAGCU |
| 714 | CAACAG |
| 715 | CCCACA |
| 716 | GGUGAA |
| 717 | ACUGCU |
| 718 | AGUCUC |
| 719 | GGCAAA |
| 720 | CCUGCA |
| 721 | AGGUUG |
| 722 | GCUGCA |
| 723 | UGAGCA |
| 724 | AGUGGA |
| 725 | AGACUG |
| 726 | AGCCCA |
| 727 | AACCUC |
| 728 | AUGCCA |
| 729 | CAAGCA |
| 730 | AACCCA |
| 731 | GCAGCA |
| 732 | ACAGGG |
| 733 | CUCCAC |
| 734 | GAGUGA |
| 735 | AGCAGC |
| 736 | CCCAAG |
| 737 | GCUGAA |
| 738 | CUUGGC |
| 739 | AAUGGG |
| 740 | CCUGAC |
| 741 | CCACAA |
| 742 | GGCCAA |
| 743 | ACACCA |
| 744 | CUGGCA |
| 745 | GUGUGA |
| 746 | UGGCAA |
| 747 | AGCCAU |
| 748 | CCAAGG |
| 749 | ACCAGC |
| 750 | AUCCUG |
| 751 | CAUCAC |
| 752 | CCCUGC |
| 753 | AUGAGG |
| 754 | CAGCCC |
| 755 | CAUGCA |
| 756 | GGCACA |
| 757 | AGAGGC |
| 758 | CCCUUC |
| 759 | AGUGUG |
| 760 | AACCUG |
| 761 | AGACAC |
| 762 | AAAGGC |
| 763 | CCACAC |
| 764 | GCAAGA |
| 765 | CCCAUC |
| 766 | CUGUGC |
| 767 | ACCCUG |
| 768 | AAAGCC |
| 769 | CUCUAC |
| 770 | ACAGGU |
| 771 | ACUGGA |
| 772 | ACAGCU |
| 473 | AGGCUG |
| 474 | CACACA |
| 475 | CCUCCC |
| 476 | CAGGAG |
| 477 | CUGGGA |
| 478 | CCCAGC |
| 479 | ACACAC |
| 480 | CUCCCA |
| 481 | CCCAGG |
| 482 | AGGCAG |
| 483 | ACAGAG |
| 484 | AGAGAG |
| 485 | CCAGGC |
| 486 | GCCUCC |
| 487 | CCAGCC |
| 488 | AGGAAG |
| 489 | AGGAGA |
| 490 | CCUCAG |
| 491 | GAGAGA |
| 492 | AGCCUG |
| 493 | GGAGAA |
| 494 | CAGAGA |
| 495 | ACUGCA |
| 496 | CUCAGC |
| 497 | AGACAG |
| 498 | CACUGC |
| 499 | AGGAGG |
| 500 | CCCAAA |
| 501 | CUGAGA |
| 502 | CUCCAG |
| 503 | CAGGCA |
| 504 | AGCUGG |
| 505 | AGUGAG |
| 506 | CACAGA |
| 507 | UCAGGA |
| 508 | CAGGAA |
| 509 | CCACCA |
| 510 | CCUGCC |
| 511 | CAGUGA |
| 512 | CUGGGC |
| 513 | CCUGGC |
| 514 | UCCCAA |
| 515 | CCAGGA |
| 516 | CAGAAG |
| 517 | CCAAAG |
| 518 | CCAGCA |
| 519 | GAGGCA |
| 520 | AUCCCA |
| 521 | AGCCUC |
| 522 | CAGUGG |
| 523 | GGCUCA |
| 524 | GACAGA |
| 525 | UGGGAA |
| 526 | GGAAGA |
| 527 | AGGGAA |
| 528 | GGGAAA |
| 529 | CUGGAA |
| 530 | UCUCCA |
| 531 | GAGGAA |
| 532 | CACCUG |
| 533 | GAAGGA |
| 534 | ACACAG |
| 535 | GGCUGA |
| 536 | GCCUCA |
| 537 | AGAGGA |
| 538 | GAGACA |
| 539 | GCUGGA |
| 540 | CUUUCC |
| 541 | CUGCAA |
| 542 | CUUCUC |
| 543 | CCUGAG |
| 544 | CCUGUA |
| 545 | CAGAGG |
| 546 | AGAUGG |
| 547 | AUGGUG |
| 548 | CACCAC |
| 549 | ACUCCA |
| 550 | ACCCAG |
| 551 | UGCCCA |
| 552 | AGAAGG |
| 553 | CUUCCA |
| 554 | AGCCAG |
| 555 | CCUUCC |
| 556 | GGAGGA |
| 557 | AGGGAG |
| 558 | CUGUGA |
| 559 | ACUCUG |
| 560 | CAGCAG |
| 561 | CCAGAA |
| 562 | GGCAGA |
| 563 | CUCAGA |
| 564 | UCUGCA |
| 565 | GUCUCA |
| 566 | CUCUGC |
| 567 | AAGGAG |
| 568 | GAGCCA |
| 569 | AGGUGG |
| 570 | CAUCUC |
| 571 | AGCAGA |
| 572 | ACAGGC |
| 773 | CAUCCA |
| 774 | ACUGGG |
| 775 | CACUCA |
| 776 | AUCCAG |
| 777 | CUGUUC |
| 778 | AUCUCC |
| 779 | CCUGUC |
| 780 | CUACAG |
| 781 | AGUGGG |
| 782 | GGUUCA |
| 783 | ACUUCC |
| 784 | AGCAUG |
| 785 | AGGGCA |
| 786 | UAGCCA |
| 787 | CUGACC |
| 788 | GAGACC |
| 789 | UGGACA |
| 790 | AAGGCU |
| 791 | CACUUC |
| 792 | AAUGGC |
| 793 | CAUCAG |
| 794 | CACUGG |
| 795 | CUGAGC |
| 796 | AUCCUC |
| 797 | CAGGCC |
| 798 | AACAGG |
| 799 | CAGAAC |
| 800 | AUCUGC |
| 801 | ACUUGG |
| 802 | CAAGAC |
| 803 | CUUGGA |
| 804 | AGCUAC |
| 805 | AGCUUC |
| 806 | GCAACA |
| 807 | ACACCU |
| 808 | ACCUGA |
| 809 | GAGCAA |
| 810 | GCCAAA |
| 811 | GAAAGC |
| 812 | CACCAA |
| 813 | CCCUGA |
| 814 | AACUGG |
| 815 | CCAUCC |
| 816 | CUUGCA |
| 817 | CAGGGC |
| 818 | GGAACA |
| 819 | CUUCAC |
| 820 | ACCACU |
| 821 | CUGACA |
| 822 | GCAGCC |
| 823 | CCAUGA |
| 824 | AAGGUG |
| 825 | CACCAG |
| 826 | ACAGAC |
| 827 | GUGACA |
| 828 | GCUUCC |
| 829 | GAACUC |
| 830 | AGACUC |
| 831 | AGUCAG |
| 832 | AAGAGC |
| 833 | CCCUCA |
| 834 | CACAUC |
| 835 | GCCACA |
| 836 | AAGCCU |
| 837 | AGUAGC |
| 838 | AGUCUG |
| 839 | CUGGUC |
| 840 | GGGCAA |
| 841 | AUGUGC |
| 842 | GAUCAC |
| 843 | CCUCAC |
| 844 | GCAUGA |
| 845 | CCUUGA |
| 846 | UGCCAA |
| 847 | CCAUGC |
| 848 | AACCAG |
| 849 | GCUUCA |
| 850 | ACAAGG |
| 851 | GUGCCA |
| 852 | CUCAUC |
| 853 | CUCACC |
| 854 | AGGCAC |
| 855 | AGCUGC |
| 856 | AGCUCC |
| 857 | GCUUGA |
| 858 | AUUCCC |
| 859 | AACAGC |
| 860 | CCAAAC |
| 861 | GAGAAC |
| 862 | AGCCCU |
| 863 | CAGUCA |
| 864 | AGGAGC |
| 865 | AUCUGG |
| 866 | CAAUGG |
| 867 | CACAAG |
| 868 | AGGGAU |
| 869 | GAAUGC |
| 870 | ACCAGA |
| 871 | AUCCAC |
| 872 | GUCCCA |
| 873 | GCCUGA |
| 874 | GCCAGA |
| 875 | GACCUC |
| 876 | AGGAAC |
| 877 | CAUGGC |
| 878 | ACCUUG |
| 879 | GAGUGC |
| 880 | AACUGC |
| 881 | ACAUGC |
| 882 | ACCCAA |
| 883 | GGAUCA |
| 884 | ACCACC |
| 885 | AAGACC |
| 886 | CAAACC |
| 887 | GGGUGA |
| 888 | GGCUAA |
| 889 | ACCAGG |
| 890 | AGGGUG |
| 891 | CAGUUC |
| 892 | GAGAUC |
| 893 | GAAGAC |
| 894 | ACAGCC |
| 895 | AUCACC |
| 896 | CAAGGC |
| 897 | ACCAUC |
| 898 | AAUGCC |
| 899 | ACCUGC |
| 900 | GAAGGC |
| 901 | AACCAC |
| 902 | GCUCCA |
| 903 | AACCCU |
| 904 | CAUUGC |
| 905 | CUGUCC |
| 906 | GGAUGA |
| 907 | GUUGCA |
| 908 | CAUGGG |
| 909 | GCUCAA |
| 910 | CAACUC |
| 911 | GCAGGC |
| 912 | GGAGCA |
| 913 | ACCAAG |
| 914 | CUACUC |
| 915 | AGCUUG |
| 916 | AUGAGC |
| 917 | AGAGCC |
| 918 | AGGACU |
| 919 | ACUCAC |
| 920 | CAUCCC |
| 921 | AAGGAC |
| 922 | GUUCCA |
| 923 | GGAGAC |
| 924 | GCUGCC |
| 925 | AGUCCC |
| 926 | GGGACA |
| 927 | UGACCA |
| 928 | CCACUC |
| 929 | GUCACA |
| 930 | CAUGUC |
| 931 | AUGUCC |
| 932 | AAGCAC |
| 933 | CAACAC |
| 934 | GAAGCC |
| 935 | UACCCA |
| 936 | GCACCA |
| 937 | AGAUGC |
| 938 | ACACUC |
| 939 | CAGUGC |
| 940 | CUCCUA |
| 941 | AGAGUC |
| 942 | ACCCAC |
| 943 | AAGCUC |
| 944 | GUCAGA |
| 945 | ACUCUC |
| 946 | GCACAC |
| 947 | AGACCU |
| 948 | CAGGUA |
| 949 | ACCUUC |
| 950 | CUUGCC |
| 951 | CUGCAG |
| 952 | GAACCA |
| 953 | UAGGCA |
| 954 | AGUCCA |
| 955 | CUAGGA |
| 956 | CAACCA |
| 957 | AGUUCC |
| 958 | AGUCAC |
| 959 | AGGCUC |
| 960 | GAAGUC |
| 961 | GAGCCC |
| 962 | AUCAGG |
| 963 | AGCAUC |
| 964 | UGGCCA |
| 965 | CAGGAC |
| 966 | GGUGCA |
| 967 | CCAGAC |
| 968 | AAGGCC |
| 969 | GUUGGA |
| 970 | GCAAAC |
| 971 | CAACUG |
| 972 | CUGAAC |
| 973 | GAGUCA |
| 974 | AUGCAC |
| 975 | GCACAA |
| 976 | CAAGGG |
| 977 | CAGAUC |
| 978 | CUGCUA |
| 979 | GCCAGC |
| 980 | AGGUGC |
| 981 | CCAGUC |
| 982 | CUACCA |
| 983 | AGUUGG |
| 984 | CCUUGC |
| 985 | CUUGUC |
| 986 | AGUGAC |
| 987 | GACUGA |
| 988 | AGGUUC |
| 989 | GAACAC |
| 990 | GACUCA |
| 991 | AUGCCC |
| 992 | CAGUAG |
| 993 | GUGCAA |
| 994 | UAGGGA |
| 995 | GGCCCA |
| 996 | AGGGAC |
| 997 | GGACAA |
| 998 | AGGUAG |
| 999 | ACUGCC |
| 1000 | ACUGUC |
| 1001 | GGGCCA |
| 1002 | AGAACC |
| 1003 | CCUAGA |
| 1004 | AGCAAC |
| 1005 | ACUCCC |
| 1006 | GAGAGC |
| 1007 | GAUGCA |
| 1008 | AGACCC |
| 1009 | ACCCAU |
| 1010 | AAGUCC |
| 1011 | AAGCCC |
| 1012 | CCUCUA |
| 1013 | GCCCAC |
| 1014 | AACACC |
| 1015 | AGGCCC |
| 1016 | AUCAGC |
| 1017 | GUCUGA |
| 1018 | ACUUGC |
| 1019 | ACAUCC |
| 1020 | CUAGAG |
| 1021 | CCAAGC |
| 1022 | GAUUCC |
| 1023 | CUGGAC |
| 1024 | GAUCCA |
| 1025 | GUGUCA |
| 1026 | GAACCC |
| 1027 | CAAGUC |
| 1028 | GCAUCA |
| 1029 | AUGCUC |
| 1030 | GGCAAC |
| 1031 | GGAGCC |
| 1032 | ACAGUC |
| 1033 | GCUCCC |
| 1034 | GGUGAC |
| 1035 | CAUUGG |
| 1036 | GACUCC |
| 1037 | CUCAAC |
| 1038 | GCAACC |
| 1039 | AGUGUC |
| 1040 | CACAAC |
| 1041 | AGCACC |
| 1042 | ACUGGC |
| 1043 | CCAGUA |
| 1044 | ACACCC |
| 1045 | AAGGGU |
| 1046 | ACCCUC |
| 1047 | GAGGGC |
| 1048 | GGUAGA |
| 1049 | ACCAAC |
| 1050 | AAGGGC |
| 1051 | AGGCUA |
| 1052 | AUGGCC |
| 1053 | ACCAGU |
| 1054 | AGGAUC |
| 1055 | CCCAAC |
| 1056 | AGGGCC |
| 1057 | CAAGCC |
| 1058 | AAGGUC |
| 1059 | GACCAA |
| 1060 | CCUACA |
| 1061 | AUUGCC |
| 1062 | CAGACC |
| 1063 | ACAAGC |
| 1064 | CAGUCC |
| 1065 | GGACAC |
| 1066 | GCCAAC |
| 1067 | GCUGUA |
| 1068 | CUGGUA |
| 1069 | GGCCAC |
| 1070 | GAUGGC |
| 1071 | CAAUGC |
| 1072 | CACCUA |
| 1073 | GAUUGC |
| 1074 | AGUGCC |
| 1075 | CAUGAC |
| 1076 | GACCCA |
| 1077 | GAGGAC |
| 1078 | CAGCUG |
| 1079 | CCCUAA |
| 1080 | CAAUCC |
| 1081 | GUGAAC |
| 1082 | CCAACC |
| 1083 | ACUGAC |
| 1084 | CAGGUC |
| 1085 | AGCCUA |
| 1086 | GAAUCC |
| 1087 | AGGGUC |
| 1088 | GACAGC |
| 1089 | CUAGCA |
| 1090 | AGUAGG |
| 1091 | GAGGUA |
| 1092 | AUGGAC |
| 1093 | GCCCAA |
| 1094 | AUUGGG |
| 1095 | AUGGGC |
| 1096 | CAUAGG |
| 1097 | GUGGUA |
| 1098 | CCUAAG |
| 1099 | GGGCAC |
| 1100 | ACAACC |
| 1101 | CUGUAC |
| 1102 | CCACUA |
| 1103 | GGCACC |
| 1104 | AGCUAG |
| 1105 | GGGAAC |
| 1106 | CUGAUC |
| 1107 | GCAGAC |
| 1108 | AUGGUC |
| 1109 | AUUGGC |
| 1110 | GGGUCA |
| 1111 | CAACCC |
| 1112 | AGAUCC |
| 1113 | CUUGAC |
| 1114 | GGACUA |
| 1115 | CCUUAG |
| 1116 | GCAGUA |
| 1117 | CCCAUA |
| 1118 | GAGCUA |
| 1119 | GAUGAC |
| 1120 | GGUUGA |
| 1121 | GCUACA |
| 1122 | GCACCC |
| 1123 | GGCUUA |
| 1124 | GGGAUA |
| 1125 | GGUCAA |
| 1126 | AGGGUA |
| 1127 | AUGACC |
| 1128 | ACCUAG |
| 1129 | GUAGGA |
| 1130 | GCUAGA |
| 1131 | CAUAGC |
| 1132 | GUCCAA |
| 1133 | AGUUGC |
| 1134 | GGUACA |
| 1135 | GAGCAC |
| 1136 | GCCAUA |
| 1137 | CCAUAG |
| 1138 | CUUAGC |
| 1139 | GAUGCC |
| 1140 | GCUCUA |
| 1141 | AGGACC |
| 1142 | CCCUUA |
| 1143 | AUAGGC |
| 1144 | CACGCC |
| 1145 | GGCAUA |
| 1146 | GCAUCC |
| 1147 | GUGCUA |
| 1148 | CCAAUC |
| 1149 | GACAUC |
| 1150 | CAGGCG |
| 1151 | GUAGCA |
| 1152 | GGACCA |
| 1153 | GACUGC |
| 1154 | GGGUAA |
| 1155 | CUAGGC |
| 1156 | GCAAGC |
| 1157 | AGGCCU |
| 1158 | GGAGUA |
| 1159 | GACCAC |
| 1160 | GGAACC |
| 1161 | ACCCUA |
| 1162 | GAGUCC |
| 1163 | GCUGAC |
| 1164 | AGGUCC |
| 1165 | CCUACC |
| 1166 | GGUCAC |
| 1167 | GCCUUA |
| 1168 | GUACCA |
| 1169 | CUUACC |
| 1170 | GACUAC |
| 1171 | CUAUGC |
| 1172 | ACCUAC |
| 1173 | CUACAC |
| 1174 | CUAGAC |
| 1175 | AUACCC |
| 1176 | GACACC |
| 1177 | CACUAC |
| 1178 | AUAGGG |
| 1179 | GGGACC |
| 1180 | CUAAGC |
| 1181 | CCCUAG |
| 1182 | AUAGCC |
| 1183 | CAUACC |
| 1184 | GCCUAA |
| 1185 | CCUUAC |
| 1186 | GGUCCA |
| 1187 | CUAUCC |
| 1188 | CCUAGC |
| 1189 | ACUAGG |
| 1190 | GACAAC |
| 1191 | CAGUAC |
| 1192 | CUAGCC |
| 1193 | CCUAAC |
| 1194 | GGUGUA |
| 1195 | AGGUAC |
| 1196 | CGGGAG |
| 1197 | CACUAG |
| 1198 | GUCCAC |
| 1199 | GAUCCC |
| 1200 | AGGCGG |
| 1201 | GGACCC |
| 1202 | CUACCC |
| 1203 | CUCGGC |
| 1204 | CCUAUC |
| 1205 | CCAUAC |
| 1206 | GGCCUA |
| 1207 | CCCUAC |
| 1208 | CCAUGG |
| 1209 | GGGUUA |
| 1210 | ACUACC |
| 1211 | GUCCUA |
| 1212 | GCACUA |
| 1213 | GCUAAC |
| 1214 | GUACAC |
| 1215 | GACCUA |
| 1216 | AGUACC |
| 1217 | CUAACC |
| 1218 | GGUCUA |
| 1219 | GGAUAC |
| 1220 | GCUUAC |
| 1221 | GGGCUA |
| 1222 | GUCAAC |
| 1223 | CGCCUC |
| 1224 | CUAGUC |
| 1225 | GAUACC |
| 1226 | GCCCUA |
| 1227 | GGUAAC |
| 1228 | GAUAGC |
| 1229 | AGACGG |
| 1230 | GGCUAC |
| 1231 | CGUCUC |
| 1232 | ACUAGC |
| 1233 | GCAUAC |
| 1234 | GGUUAC |
| 1235 | GAGUAC |
| 1236 | CGAGAC |
| 1237 | GUAGAC |
| 1238 | AGGCCG |
| 1239 | GCCUAC |
| 1240 | GCUACC |
| 1241 | CGCCCA |
| 1242 | GCGUGA |
| 1243 | GAGCUC |
| 1244 | CCUAGG |
| 1245 | ACGCCU |
| 1246 | CCGAGG |
| 1247 | GGCGGA |
| 1248 | CGCCAC |
| 1249 | GGGUAC |
| 1250 | CGUGAG |
| 1251 | ACCACG |
| 1252 | GCAUGC |
| 1253 | CCACCG |
| 1254 | CCACGC |
| 1255 | CCGAGA |
| 1256 | CCCGAG |
| 1257 | CGGCUC |
| 1258 | CCGGGA |
| 1259 | UCGAGA |
| 1260 | GUGCAC |
| 1261 | CGAGGC |
| 1262 | CCUCCG |
| 1263 | GGCCGA |
| 1264 | CGGGCA |
| 1265 | ACCCGG |
| 1266 | AGCCGA |
| 1267 | CUCCGC |
| 1268 | AUCUCG |
| 1269 | CCCGUC |
| 1270 | CGUGCC |
| 1271 | AUCACG |
| 1272 | AGCCGG |
| 1273 | CACACG |
| 1274 | CACCGC |
| 1275 | UCCCGA |
| 1276 | AGAUCG |
| 1277 | CACCCG |
| 1278 | CGUUUC |
| 1279 | ACCUCG |
| 1280 | ACGUGG |
| 1281 | ACGGUG |
| 1282 | ACGGAG |
| 1283 | AAGCGA |
| 1284 | AAACGG |
| 1285 | AGCGAG |
| 1286 | AGGCGC |
| 1287 | CAAGCG |
| 1288 | CUCGAA |
| 1289 | ACUCGG |
| 1290 | ACCGUG |
| 1291 | AGAGCG |
| 1292 | AGCGAU |
| 1293 | CCGCCA |
| 1294 | CACGAG |
| 1295 | ACACGU |
| 1296 | AUGGCG |
| 1297 | AACGUG |
| 1298 | CCCACG |
| 1299 | CGUGAA |
| 1300 | ACACGG |
| 1301 | ACGAGG |
| 1302 | AACACG |
| 1303 | GCGCCA |
| 1304 | ACGCCA |
| 1305 | UCACGA |
| 1306 | GGAUCC |
| 1307 | GCGAGA |
| 1308 | AACCCG |
| 1309 | CAGCCG |
| 1310 | ACGUGA |
| 1311 | ACGCCC |
| 1312 | GAGCGA |
| 1313 | CCACGG |
| 1314 | GACGGA |
| 1315 | CGAUUC |
| 1316 | ACGGGA |
| 1317 | CGGCCA |
| 1318 | GGCGCA |
| 1319 | ACUCCG |
| 1320 | CACGCA |
| 1321 | GGGCGA |
| 1322 | AAUCGC |
| 1323 | AAAGCG |
| 1324 | CGGGAA |
| 1325 | CGGUGA |
| 1326 | CGUGGC |
| 1327 | CGCUGC |
| 1328 | ACGAAG |
| 1329 | ACGUGC |
| 1330 | CCAGCG |
| 1331 | AGUUCG |
| 1332 | CGCACC |
| 1333 | CGUGGA |
| 1334 | CGACAG |
| 1335 | CGGCAG |
| 1336 | CCGCAG |
| 1337 | CGAGUA |
| 1338 | GUUCGA |
| 1339 | AACGGG |
| 1340 | CGUGCA |
| 1341 | ACGCUG |
| 1342 | CAGACG |
| 1343 | CCAACG |
| 1344 | GCGGGA |
| 1345 | CAGCGG |
| 1346 | CGGAGC |
| 1347 | AGCGUG |
| 1348 | CGCUCC |
| 1349 | CUGCGC |
| 1350 | AUCGUG |
| 1351 | CCGUGC |
| 1352 | CCUGCG |
| 1353 | ACCGCA |
| 1354 | ACGGAA |
| 1355 | CACGGC |
| 1356 | AGCCCG |
| 1357 | CACGGG |
| 1358 | CACGGA |
| 1359 | CAACGA |
| 1360 | CACGUA |
| 1361 | AGCGGG |
| 1362 | CGGAAG |
| 1363 | CGAGAA |
| 1364 | CGCACA |
| 1365 | AGAACG |
| 1366 | CGCAGC |
| 1367 | CAAACG |
| 1368 | ACGCAG |
| 1369 | AAGACG |
| 1370 | GGUACC |
| 1371 | CCGGAG |
| 1372 | CGAGGA |
| 1373 | GAUCGC |
| 1374 | ACCCGC |
| 1375 | CACGUG |
| 1376 | GCUAGC |
| 1377 | ACACGC |
| 1378 | AAGGCG |
| 1379 | CGGCUA |
| 1380 | ACUGCG |
| 1381 | ACGUCU |
| 1382 | CAGCGA |
| 1383 | CACGUC |
| 1384 | ACGUUG |
| 1385 | GCGACA |
| 1386 | AUCCCG |
| 1387 | AGGGCG |
| 1388 | CCUCGC |
| 1389 | CGGAGA |
| 1390 | CGCCAG |
| 1391 | CCGUGA |
| 1392 | ACGAGA |
| 1393 | ACAGCG |
| 1394 | AGCACG |
| 1395 | AUCCGC |
| 1396 | UCGCCA |
| 1397 | CUCGCC |
| 1398 | CCUUCG |
| 1399 | CCACGA |
| 1400 | CAGCGC |
| 1401 | CCCUCG |
| 1402 | CCCGGA |
| 1403 | CGAGAG |
| 1404 | GGCGAC |
| 1405 | CCCGCA |
| 1406 | CACGAA |
| 1407 | AGACGA |
| 1408 | CGAAGA |
| 1409 | AGCCGC |
| 1410 | CGGAAA |
| 1411 | AUCGAG |
| 1412 | CGCAGA |
| 1413 | CGGAUC |
| 1414 | CCGCAC |
| 1415 | CGAAAC |
| 1416 | AUCGCA |
| 1417 | AAACCG |
| 1418 | GCACGC |
| 1419 | CAUUCG |
| 1420 | CGUGUC |
| 1421 | CGUUCA |
| 1422 | AACGGA |
| 1423 | GCACGA |
| 1424 | ACGCAC |
| 1425 | ACGAUG |
| 1426 | AGGACG |
| 1427 | AACGCA |
| 1428 | CGCUUC |
| 1429 | CGUCCC |
| 1430 | AAGCGG |
| 1431 | AGGCGA |
| 1432 | ACACGA |
| 1433 | ACGGCA |
| 1434 | CCUCGA |
| 1435 | ACGUCA |
| 1436 | CCGCUC |
| 1437 | CAUGCG |
| 1438 | AAGCCG |
| 1439 | AACGAG |
| 1440 | CCGGCA |
| 1441 | AAGCGC |
| 1442 | GAGCGC |
| 1443 | AAACGC |
| 1444 | ACGUUC |
| 1445 | ACCGAG |
| 1446 | CGCUGA |
| 1447 | CCGAGC |
| 1448 | CCGUCC |
| 1449 | CGCUCA |
| 1450 | GCGAAA |
| 1451 | GCGGCA |
| 1452 | CCGAAG |
| 1453 | AACGCU |
| 1454 | ACACCG |
| 1455 | AGCGGC |
| 1456 | CGAAAG |
| 1457 | CUCGGA |
| 1458 | CGUGUA |
| 1459 | AGCGAA |
| 1460 | CCAUCG |
| 1461 | CCCGAC |
| 1462 | AGCGCC |
| 1463 | CGUUCC |
| 1464 | AUGACG |
| 1465 | CGAGCC |
| 1466 | CACUCG |
| 1467 | CUGCGA |
| 1468 | CUCGUC |
| 1469 | AAGCGU |
| 1470 | ACAACG |
| 1471 | GCCGCA |
| 1472 | AACUCG |
| 1473 | CCGAAA |
| 1474 | CGUCCA |
| 1475 | CUCCGA |
| 1476 | ACGAUC |
| 1477 | ACGUCC |
| 1478 | ACCGGG |
| 1479 | CACCGG |
| 1480 | CGGCAC |
| 1481 | AACGUC |
| 1482 | AGACGC |
| 1483 | CCGGAA |
| 1484 | CGUCAG |
| 1485 | ACUUCG |
| 1486 | ACGGCU |
| 1487 | CACCGA |
| 1488 | AGCGGA |
| 1489 | CGUGAC |
| 1490 | CGCAAA |
| 1491 | AGGUCG |
| 1492 | CGAUGA |
| 1493 | ACGUAG |
| 1494 | CGUCAC |
| 1495 | CCCGAA |
| 1496 | GAUCGA |
| 1497 | CGAGCA |
| 1498 | CGGUGC |
| 1499 | AGCGCA |
| 1500 | AUGCGG |
| 1501 | CAUACG |
| 1502 | CCGUCA |
| 1503 | AGACCG |
| 1504 | UCGUCA |
| 1505 | ACGCAU |
| 1506 | ACGGGC |
| 1507 | GGCGAA |
| 1508 | GAACGA |
| 1509 | AGCUCG |
| 1510 | ACGACA |
| 1511 | CCGCAA |
| 1512 | ACGGCC |
| 1513 | GCGCAC |
| 1514 | ACAUCG |
| 1515 | GCCGGA |
| 1516 | CAUCGC |
| 1517 | UCCGCA |
| 1518 | CGGGAC |
| 1519 | CAUCGA |
| 1520 | ACCGCC |
| 1521 | AUUCCG |
| 1522 | CAUCCG |
| 1523 | ACCCGU |
| 1524 | CUUCGA |
| 1525 | UCGGAA |
| 1526 | ACGCAA |
| 1527 | AUUGCG |
| 1528 | ACGAGU |
| 1529 | GAACGC |
| 1530 | ACCCGA |
| 1531 | AUUCGG |
| 1532 | UCGGCA |
| 1533 | AAUGCG |
| 1534 | CCGACC |
| 1535 | CCGUUC |
| 1536 | AUGCCG |
| 1537 | AUCGCC |
| 1538 | ACCGAA |
| 1539 | AACCGA |
| 1540 | UCCGAA |
| 1541 | ACCGCU |
| 1542 | CGUAAG |
| 1543 | ACUCGA |
| 1544 | CGUUGA |
| 1545 | AACCGU |
| 1546 | GCGUCC |
| 1547 | CUCGCA |
| 1548 | GCGGAA |
| 1549 | CGGCAA |
| 1550 | CGAAGC |
| 1551 | GCCCGA |
| 1552 | ACGCUC |
| 1553 | AGCGUC |
| 1554 | GUGCGA |
| 1555 | CGGACA |
| 1556 | AACGGU |
| 1557 | CGAACA |
| 1558 | ACGGAU |
| 1559 | GACGCA |
| 1560 | CUUCGC |
| 1561 | AGUGCG |
| 1562 | CCGUAA |
| 1563 | CGGGUC |
| 1564 | CGUAGA |
| 1565 | CGUUGC |
| 1566 | CGCCAA |
| 1567 | AGCGCU |
| 1568 | GACGAA |
| 1569 | GACGCC |
| 1570 | CGAGUC |
| 1571 | ACCGUA |
| 1572 | CGAUCC |
| 1573 | GCCGAA |
| 1574 | AUACGG |
| 1575 | CGAUCA |
| 1576 | CGCAAG |
| 1577 | GCGAGC |
| 1578 | AAGUCG |
| 1579 | CACGAC |
| 1580 | CCGACA |
| 1581 | AACGCC |
| 1582 | ACGGUA |
| 1583 | UACGCA |
| 1584 | AGUCGG |
| 1585 | ACUACG |
| 1586 | CGGUUC |
| 1587 | AUGCGC |
| 1588 | ACCGCG |
| 1589 | AAUCCG |
| 1590 | GACGGC |
| 1591 | ACCGUC |
| 1592 | CAGUCG |
| 1593 | CGCCUA |
| 1594 | CGUCUA |
| 1595 | ACGUAC |
| 1596 | CGCAUC |
| 1597 | CGGUCA |
| 1598 | CGUACA |
| 1599 | CAACGG |
| 1600 | UGCGAA |
| 1601 | CGAUGC |
| 1602 | AUGUCG |
| 1603 | CGACUC |
| 1604 | CCUACG |
| 1605 | GGACGA |
| 1606 | ACCGGC |
| 1607 | CGACAA |
| 1608 | UACGGA |
| 1609 | CCGGAC |
| 1610 | AACGGC |
| 1611 | CGUCAA |
| 1612 | CGUAUC |
| 1613 | AACCGC |
| 1614 | CAUCGG |
| 1615 | CGUUAG |
| 1616 | AUGCGA |
| 1617 | GCGCAA |
| 1618 | AAUCGG |
| 1619 | ACGAGC |
| 1620 | AUCGUC |
| 1621 | CUCGAC |
| 1622 | AGUCCG |
| 1623 | UCGCAA |
| 1624 | GCGUCA |
| 1625 | ACGACU |
| 1626 | AACCGG |
| 1627 | CGGUCC |
| 1628 | CGGGUA |
| 1629 | ACGGAC |
| 1630 | CGACCC |
| 1631 | UCGACA |
| 1632 | CGACAC |
| 1633 | ACGAAC |
| 1634 | GCUCGA |
| 1635 | CUACGA |
| 1636 | CCGGUC |
| 1637 | CGAAUC |
| 1638 | CAACGC |
| 1639 | AGCGAC |
| 1640 | CCGUAG |
| 1641 | ACUCGC |
| 1642 | AUCGGG |
| 1643 | CGGUAA |
| 1644 | AACGAC |
| 1645 | AGUCGA |
| 1646 | AUCCGG |
| 1647 | CCCGUA |
| 1648 | ACGCUA |
| 1649 | CAACCG |
| 1650 | ACGGUC |
| 1651 | GACGUA |
| 1652 | AUAGCG |
| 1653 | CGACCA |
| 1654 | CAAUCG |
| 1655 | CGGAAC |
| 1656 | CGGUAG |
| 1657 | CUCGUA |
| 1658 | AUCCGA |
| 1659 | GACCGA |
| 1660 | ACCGGA |
| 1661 | CGCAUA |
| 1662 | AUACGC |
| 1663 | AGCGUA |
| 1664 | GACCGC |
| 1665 | CGCUUA |
| 1666 | CGCUAA |
| 1667 | CCGUUA |
| 1668 | AUUCGC |
| 1669 | CGUAGC |
| 1670 | UAGCGA |
| 1671 | CGCAAC |
| 1672 | CGGACC |
| 1673 | AUCGGA |
| 1674 | CGUAAC |
| 1675 | CGAUAG |
| 1676 | GCCGAC |
| 1677 | CUCGAG |
| 1678 | ACCGAU |
| 1679 | AUACCG |
| 1680 | CUACGC |
| 1681 | AGUACG |
| 1682 | GUCGAA |
| 1683 | GGCGUA |
| 1684 | GCGGAC |
| 1685 | UACCGA |
| 1686 | CCGCUA |
| 1687 | GGUCGA |
| 1688 | GCGAUA |
| 1689 | ACGACC |
| 1690 | AGUCGC |
| 1691 | GCGCGA |
| 1692 | CGUUAC |
| 1693 | AUCGGC |
| 1694 | CGGAUA |
| 1695 | GCGUAA |
| 1696 | CGAACC |
| 1697 | GCGUUA |
| 1698 | GACGAC |
| 1699 | CGCUAC |
| 1700 | GCGACC |
| 1701 | CGGUUA |
| 1702 | CCGAAC |
| 1703 | GUCGGA |
| 1704 | GUCGCA |
| 1705 | GCCGUA |
| 1706 | CCGGUA |
| 1707 | CGUACC |
| 1708 | ACCGAC |
| 1709 | CCGAUC |
| 1710 | AUCGCG |
| 1711 | CGCUAG |
| 1712 | GUCCGA |
| 1713 | UCCGGA |
| 1714 | CCGAUA |
| 1715 | AUCGAC |
| 1716 | GCGGUA |
| 1717 | GCGCUA |
| 1718 | CACGCG |
| 1719 | GUACGA |
| 1720 | UGCGCA |
| 1721 | GUCGUA |
| 1722 | CGACUA |
| 1723 | CCGUAC |
| 1724 | CGAUAC |
| 1725 | CGGUAC |
| 1726 | CGCACG |
| 1727 | GCGAAC |
| 1728 | CGCGCA |
| 1729 | AGCGCG |
| 1730 | ACGCGG |
| 1731 | ACGGCG |
| 1732 | GACGUC |
| 1733 | CGAGCG |
| 1734 | CGGACG |
| 1735 | ACGCCG |
| 1736 | ACGCGC |
| 1737 | GCGUAC |
| 1738 | CGCGAG |
| 1739 | CGCGGA |
| 1740 | CGCGUC |
| 1741 | CGGCGA |
| 1742 | CCGCGA |
| 1743 | CGCCGA |
| 1744 | ACCGGU |
| 1745 | CCGACG |
| 1746 | CCGUCG |
| 1747 | CGUCGC |
| 1748 | CGACGC |
| 1749 | ACGACG |
| 1750 | AACGCG |
| 1751 | ACGUCG |
| 1752 | CGCGAC |
| 1753 | CGAACG |
| 1754 | CGACCG |
| 1755 | ACGCGA |
| 1756 | CGCGAA |
| 1757 | CGACGA |
| 1758 | GUCGAC |
| 1759 | CGUCGA |
| 1760 | CGCGUA |
| 1761 | ACGCGU |
| 1762 | UCGCGA |
| 1763 | CGAUCG |
| 1764 | CGUACG |
| 1765 | AGGCUG |
| 1766 | CACACA |
| 1767 | CCUCCC |
| 1768 | CAGGAG |
| 1769 | CUGGGA |
| 1770 | CCCAGC |
| 1771 | ACACAC |
| 1772 | CUCCCA |
| 1773 | CCCAGG |
| 1774 | AGGCAG |
| 1775 | ACAGAG |
| 1776 | AGAGAG |
| 1777 | CCAGGC |
| 1778 | GCCUCC |
| 1779 | CCAGCC |
| 1780 | AGGAAG |
| 1781 | AGGAGA |
| 1782 | CCUCAG |
| 1783 | GAGAGA |
| 1784 | AGCCUG |
| 1785 | GGAGAA |
| 1786 | CAGAGA |
| 1787 | ACUGCA |
| 1788 | CUCAGC |
| 1789 | AGACAG |
| 1790 | CACUGC |
| 1791 | AGGAGG |
| 1792 | CCCAAA |
| 1793 | CUGAGA |
| 1794 | CUCCAG |
| 1795 | CAGGCA |
| 1796 | AGCUGG |
| 1797 | AGUGAG |
| 1798 | CACAGA |
| 1799 | UCAGGA |
| 1800 | CAGGAA |
| 1801 | CCACCA |
| 1802 | CCUGCC |
| 1803 | CAGUGA |
| 1804 | CUGGGC |
| 1805 | CCUGGC |
| 1806 | UCCCAA |
| 1807 | CCAGGA |
| 1808 | CAGAAG |
| 1809 | CCAAAG |
| 1810 | CCAGCA |
| 1811 | GAGGCA |
| 1812 | AUCCCA |
| 1813 | AGCCUC |
| 1814 | CAGUGG |
| 1815 | GGCUCA |
| 1816 | GACAGA |
| 1817 | UGGGAA |
| 1818 | GGAAGA |
| 1819 | AGGGAA |
| 1820 | GGGAAA |
| 1821 | CUGGAA |
| 1822 | UCUCCA |
| 1823 | GAGGAA |
| 1824 | CACCUG |
| 1825 | GAAGGA |
| 1826 | ACACAG |
| 1827 | GGCUGA |
| 1828 | GCCUCA |
| 1829 | AGAGGA |
| 1830 | GAGACA |
| 1831 | GCUGGA |
| 1832 | CUUUCC |
| 1833 | CUGCAA |
| 1834 | CUUCUC |
| 1835 | CCUGAG |
| 1836 | CCUGUA |
| 1837 | CAGAGG |
| 1838 | AGAUGG |
| 1839 | AUGGUG |
| 1840 | CACCAC |
| 1841 | ACUCCA |
| 1842 | ACCCAG |
| 1843 | UGCCCA |
| 1844 | AGAAGG |
| 1845 | CUUCCA |
| 1846 | AGCCAG |
| 1847 | CCUUCC |
| 1848 | GGAGGA |
| 1849 | AGGGAG |
| 1850 | CUGUGA |
| 1851 | ACUCUG |
| 1852 | CAGCAG |
| 1853 | CCAGAA |
| 1854 | GGCAGA |
| 1855 | CUCAGA |
| 1856 | UCUGCA |
| 1857 | GUCUCA |
| 1858 | CUCUGC |
| 1859 | AAGGAG |
| 1860 | GAGCCA |
| 1861 | AGGUGG |
| 1862 | CAUCUC |
| 1863 | AGCAGA |
| 1864 | ACAGGC |
| 1865 | AAGCAG |
| 1866 | CUUCCC |
| 1867 | CCUUUC |
| 1868 | AAUCCC |
| 1869 | AGAGCA |
| 1870 | GCAGGA |
| 1871 | GCAGAA |
| 1872 | UCCUCA |
| 1873 | CCCACC |
| 1874 | CUCUUC |
| 1875 | CCUCCA |
| 1876 | CAGCCA |
| 1877 | AAGGGA |
| 1878 | CUCACA |
| 1879 | AAGGCA |
| 1880 | AGAGAC |
| 1881 | CAGGGA |
| 1882 | CAGCAC |
| 1883 | ACAGCA |
| 1884 | ACAUGG |
| 1885 | CUGGCC |
| 1886 | UCACCA |
| 1887 | ACAGUG |
| 1888 | AAGAGG |
| 1889 | CACCCA |
| 1890 | CUCUGA |
| 1891 | GGGAGA |
| 1892 | AGACCA |
| 1893 | AGCCAC |
| 1894 | AGCACU |
| 1895 | ACCACA |
| 1896 | CACAUG |
| 1897 | CUCCUC |
| 1898 | CAGAGC |
| 1899 | CAGCAA |
| 1900 | AAGCCA |
| 1901 | CCCAGA |
| 1902 | CCACAG |
| 1903 | CUGAAG |
| 1904 | CAGCUA |
| 1905 | GCUCAC |
| 1906 | CUCUCC |
| 1907 | AGGCAU |
| 1908 | ACCUCA |
| 1909 | CCCUCC |
| 1910 | CCUGGA |
| 1911 | ACCAUG |
| 1912 | CAGACA |
| 1913 | CCUCUC |
| 1914 | UCCAGA |
| 1915 | AGCUGA |
| 1916 | CAAAGG |
| 1917 | AUGGAG |
| 1918 | AGAGUG |
| 1919 | GUGAGA |
| 1920 | AGGCCA |
| 1921 | CUGCAC |
| 1922 | ACUCCU |
| 1923 | AAACCC |
| 1924 | AAAGGG |
| 1925 | UCAGCA |
| 1926 | CCAGAG |
| 1927 | CCAACA |
| 1928 | AGCCAA |
| 1929 | GAGGGA |
| 1930 | GAAGCA |
| 1931 | CAGAUG |
| 1932 | GAGGCC |
| 1933 | ACUGUG |
| 1934 | CAUGCC |
| 1935 | CCUGAA |
| 1936 | CCAAGA |
| 1937 | CUCUCA |
| 1938 | UGCACA |
| 1939 | AUGCUG |
| 1940 | CCUCAA |
| 1941 | AACUCC |
| 1942 | CUGCCA |
| 1943 | CACUCC |
| 1944 | AGGUGA |
| 1945 | CACAGG |
| 1946 | AGCAAG |
| 1947 | AAGCUG |
| 1948 | ACAGGA |
| 1949 | CACCUC |
| 1950 | AGCAGG |
| 1951 | AGGUCA |
| 1952 | GCCACC |
| 1953 | AGUGCA |
| 1954 | AGAAGC |
| 1955 | AGGCAA |
| 1956 | ACACUG |
| 1957 | CCAUUC |
| 1958 | CACAGC |
| 1959 | CUGCCC |
| 1960 | CUCAAG |
| 1961 | ACUCAG |
| 1962 | CUGCUC |
| 1963 | UCCACA |
| 1964 | AUGGGA |
| 1965 | CAAGUG |
| 1966 | GGAAAC |
| 1967 | AGGACA |
| 1968 | CAGCUC |
| 1969 | CAAGGA |
| 1970 | GAAACC |
| 1971 | AUGGCA |
| 1972 | CCUUCA |
| 1973 | AGCUCA |
| 1974 | CUGUCA |
| 1975 | CAUGAG |
| 1976 | CACACC |
| 1977 | CUCCAA |
| 1978 | GACACA |
| 1979 | ACCUGG |
| 1980 | CACUGA |
| 1981 | AAGUGG |
| 1982 | CAAGAG |
| 1983 | AUGUGG |
| 1984 | AGGAUG |
| 1985 | ACCUCC |
| 1986 | GAUGGA |
| 1987 | AGAGGG |
| 1988 | CCACCC |
| 1989 | CCAGGG |
| 1990 | CAUGGA |
| 1991 | ACCUCU |
| 1992 | GUGGGA |
| 1993 | GUGGCA |
| 1994 | CAUUCC |
| 1995 | ACUGAG |
| 1996 | AGUGGC |
| 1997 | AGCACA |
| 1998 | CCAUCA |
| 1999 | AUGCAG |
| 2000 | AAGUGC |
| 2001 | CAAAGC |
| 2002 | CUUUGC |
| 2003 | GGUGGA |
| 2004 | GUGGAA |
| 2005 | AGAGCU |
| 2006 | CAACAG |
| 2007 | CCCACA |
| 2008 | GGUGAA |
| 2009 | ACUGCU |
| 2010 | AGUCUC |
| 2011 | GGCAAA |
| 2012 | CCUGCA |
| 2013 | AGGUUG |
| 2014 | GCUGCA |
| 2015 | UGAGCA |
| 2016 | AGUGGA |
| 2017 | AGACUG |
| 2018 | AGCCCA |
| 2019 | AACCUC |
| 2020 | AUGCCA |
| 2021 | CAAGCA |
| 2022 | AACCCA |
| 2023 | GCAGCA |
| 2024 | ACAGGG |
| 2025 | CUCCAC |
| 2026 | GAGUGA |
| 2027 | AGCAGC |
| 2028 | CCCAAG |
| 2029 | GCUGAA |
| 2030 | CUUGGC |
| 2031 | AAUGGG |
| 2032 | CCUGAC |
| 2033 | CCACAA |
| 2034 | GGCCAA |
| 2035 | ACACCA |
| 2036 | CUGGCA |
| 2037 | GUGUGA |
| 2038 | UGGCAA |
| 2039 | AGCCAU |
| 2040 | CCAAGG |
| 2041 | ACCAGC |
| 2042 | AUCCUG |
| 2043 | CAUCAC |
| 2044 | CCCUGC |
| 2045 | AUGAGG |
| 2046 | CAGCCC |
| 2047 | CAUGCA |
| 2048 | GGCACA |
| 2049 | AGAGGC |
| 2050 | CCCUUC |
| 2051 | AGUGUG |
| 2052 | AACCUG |
| 2053 | AGACAC |
| 2054 | AAAGGC |
| 2055 | CCACAC |
| 2056 | GCAAGA |
| 2057 | CCCAUC |
| 2058 | CUGUGC |
| 2059 | ACCCUG |
| 2060 | AAAGCC |
| 2061 | CUCUAC |
| 2062 | ACAGGU |
| 2063 | ACUGGA |
| 2064 | ACAGCU |
| 2065 | CAUCCA |
| 2066 | ACUGGG |
| 2067 | CACUCA |
| 2068 | AUCCAG |
| 2069 | CUGUUC |
| 2070 | AUCUCC |
| 2071 | CCUGUC |
| 2072 | CUACAG |
| 2073 | AGUGGG |
| 2074 | GGUUCA |
| 2075 | ACUUCC |
| 2076 | AGCAUG |
| 2077 | AGGGCA |
| 2078 | UAGCCA |
| 2079 | CUGACC |
| 2080 | GAGACC |
| 2081 | UGGACA |
| 2082 | AAGGCU |
| 2083 | CACUUC |
| 2084 | AAUGGC |
| 2085 | CAUCAG |
| 2086 | CACUGG |
| 2087 | CUGAGC |
| 2088 | AUCCUC |
| 2089 | CAGGCC |
| 2090 | AACAGG |
| 2091 | CAGAAC |
| 2092 | AUCUGC |
| 2093 | ACUUGG |
| 2094 | CAAGAC |
| 2095 | CUUGGA |
| 2096 | AGCUAC |
| 2097 | AGCUUC |
| 2098 | GCAACA |
| 2099 | ACACCU |
| 2100 | ACCUGA |
| 2101 | GAGCAA |
| 2102 | GCCAAA |
| 2103 | GAAAGC |
| 2104 | CACCAA |
| 2105 | CCCUGA |
| 2106 | AACUGG |
| 2107 | CCAUCC |
| 2108 | CUUGCA |
| 2109 | CAGGGC |
| 2110 | GGAACA |
| 2111 | CUUCAC |
| 2112 | ACCACU |
| 2113 | CUGACA |
| 2114 | GCAGCC |
| 2115 | CCAUGA |
| 2116 | AAGGUG |
| 2117 | CACCAG |
| 2118 | ACAGAC |
| 2119 | GUGACA |
| 2120 | GCUUCC |
| 2121 | GAACUC |
| 2122 | AGACUC |
| 2123 | AGUCAG |
| 2124 | AAGAGC |
| 2125 | CCCUCA |
| 2126 | CACAUC |
| 2127 | GCCACA |
| 2128 | AAGCCU |
| 2129 | AGUAGC |
| 2130 | AGUCUG |
| 2131 | CUGGUC |
| 2132 | GGGCAA |
| 2133 | AUGUGC |
| 2134 | GAUCAC |
| 2135 | CCUCAC |
| 2136 | GCAUGA |
| 2137 | CCUUGA |
| 2138 | UGCCAA |
| 2139 | CCAUGC |
| 2140 | AACCAG |
| 2141 | GCUUCA |
| 2142 | ACAAGG |
| 2143 | GUGCCA |
| 2144 | CUCAUC |
| 2145 | CUCACC |
| 2146 | AGGCAC |
| 2147 | AGCUGC |
| 2148 | AGCUCC |
| 2149 | GCUUGA |
| 2150 | AUUCCC |
| 2151 | AACAGC |
| 2152 | CCAAAC |
| 2153 | GAGAAC |
| 2154 | AGCCCU |
| 2155 | CAGUCA |
| 2156 | AGGAGC |
| 2157 | AUCUGG |
| 2158 | CAAUGG |
| 2159 | CACAAG |
| 2160 | AGGGAU |
| 2161 | GAAUGC |
| 2162 | ACCAGA |
| 2163 | AUCCAC |
| 2164 | GUCCCA |
| 2165 | GCCUGA |
| 2166 | GCCAGA |
| 2167 | GACCUC |
| 2168 | AGGAAC |
| 2169 | CAUGGC |
| 2170 | ACCUUG |
| 2171 | GAGUGC |
| 2172 | AACUGC |
| 2173 | ACAUGC |
| 2174 | ACCCAA |
| 2175 | GGAUCA |
| 2176 | ACCACC |
| 2177 | AAGACC |
| 2178 | CAAACC |
| 2179 | GGGUGA |
| 2180 | GGCUAA |
| 2181 | ACCAGG |
| 2182 | AGGGUG |
| 2183 | CAGUUC |
| 2184 | GAGAUC |
| 2185 | GAAGAC |
| 2186 | ACAGCC |
| 2187 | AUCACC |
| 2188 | CAAGGC |
| 2189 | ACCAUC |
| 2190 | AAUGCC |
| 2191 | ACCUGC |
| 2192 | GAAGGC |
| 2193 | AACCAC |
| 2194 | GCUCCA |
| 2195 | AACCCU |
| 2196 | CAUUGC |
| 2197 | CUGUCC |
| 2198 | GGAUGA |
| 2199 | GUUGCA |
| 2200 | CAUGGG |
| 2201 | GCUCAA |
| 2202 | CAACUC |
| 2203 | GCAGGC |
| 2204 | GGAGCA |
| 2205 | ACCAAG |
| 2206 | CUACUC |
| 2207 | AGCUUG |
| 2208 | AUGAGC |
| 2209 | AGAGCC |
| 2210 | AGGACU |
| 2211 | ACUCAC |
| 2212 | CAUCCC |
| 2213 | AAGGAC |
| 2214 | GUUCCA |
| 2215 | GGAGAC |
| 2216 | GCUGCC |
| 2217 | AGUCCC |
| 2218 | GGGACA |
| 2219 | UGACCA |
| 2220 | CCACUC |
| 2221 | GUCACA |
| 2222 | CAUGUC |
| 2223 | AUGUCC |
| 2224 | AAGCAC |
| 2225 | CAACAC |
| 2226 | GAAGCC |
| 2227 | UACCCA |
| 2228 | GCACCA |
| 2229 | AGAUGC |
| 2230 | ACACUC |
| 2231 | CAGUGC |
| 2232 | CUCCUA |
| 2233 | AGAGUC |
| 2234 | ACCCAC |
| 2235 | AAGCUC |
| 2236 | GUCAGA |
| 2237 | ACUCUC |
| 2238 | GCACAC |
| 2239 | AGACCU |
| 2240 | CAGGUA |
| 2241 | ACCUUC |
| 2242 | CUUGCC |
| 2243 | CUGCAG |
| 2244 | GAACCA |
| 2245 | UAGGCA |
| 2246 | AGUCCA |
| 2247 | CUAGGA |
| 2248 | CAACCA |
| 2249 | AGUUCC |
| 2250 | AGUCAC |
| 2251 | AGGCUC |
| 2252 | GAAGUC |
| 2253 | GAGCCC |
| 2254 | AUCAGG |
| 2255 | AGCAUC |
| 2256 | UGGCCA |
| 2257 | CAGGAC |
| 2258 | GGUGCA |
| 2259 | CCAGAC |
| 2260 | AAGGCC |
| 2261 | GUUGGA |
| 2262 | GCAAAC |
| 2263 | CAACUG |
| 2264 | CUGAAC |
| 2265 | GAGUCA |
| 2266 | AUGCAC |
| 2267 | GCACAA |
| 2268 | CAAGGG |
| 2269 | CAGAUC |
| 2270 | CUGCUA |
| 2271 | GCCAGC |
| 2272 | AGGUGC |
| 2273 | CCAGUC |
| 2274 | CUACCA |
| 2275 | AGUUGG |
| 2276 | CCUUGC |
| 2277 | CUUGUC |
| 2278 | AGUGAC |
| 2279 | GACUGA |
| 2280 | AGGUUC |
| 2281 | GAACAC |
| 2282 | GACUCA |
| 2283 | AUGCCC |
| 2284 | CAGUAG |
| 2285 | GUGCAA |
| 2286 | UAGGGA |
| 2287 | GGCCCA |
| 2288 | AGGGAC |
| 2289 | GGACAA |
| 2290 | AGGUAG |
| 2291 | ACUGCC |
| 2292 | ACUGUC |
| 2293 | GGGCCA |
| 2294 | AGAACC |
| 2295 | CCUAGA |
| 2296 | AGCAAC |
| 2297 | ACUCCC |
| 2298 | GAGAGC |
| 2299 | GAUGCA |
| 2300 | AGACCC |
| 2301 | ACCCAU |
| 2302 | AAGUCC |
| 2303 | AAGCCC |
| 2304 | CCUCUA |
| 2305 | GCCCAC |
| 2306 | AACACC |
| 2307 | AGGCCC |
| 2308 | AUCAGC |
| 2309 | GUCUGA |
| 2310 | ACUUGC |
| 2311 | ACAUCC |
| 2312 | CUAGAG |
| 2313 | CCAAGC |
| 2314 | GAUUCC |
| 2315 | CUGGAC |
| 2316 | GAUCCA |
| 2317 | GUGUCA |
| 2318 | GAACCC |
| 2319 | CAAGUC |
| 2320 | GCAUCA |
| 2321 | AUGCUC |
| 2322 | GGCAAC |
| 2323 | GGAGCC |
| 2324 | ACAGUC |
| 2325 | GCUCCC |
| 2326 | GGUGAC |
| 2327 | CAUUGG |
| 2328 | GACUCC |
| 2329 | CUCAAC |
| 2330 | GCAACC |
| 2331 | AGUGUC |
| 2332 | CACAAC |
| 2333 | AGCACC |
| 2334 | ACUGGC |
| 2335 | CCAGUA |
| 2336 | ACACCC |
| 2337 | AAGGGU |
| 2338 | ACCCUC |
| 2339 | GAGGGC |
| 2340 | GGUAGA |
| 2341 | ACCAAC |
| 2342 | AAGGGC |
| 2343 | AGGCUA |
| 2344 | AUGGCC |
| 2345 | ACCAGU |
| 2346 | AGGAUC |
| 2347 | CCCAAC |
| 2348 | AGGGCC |
| 2349 | CAAGCC |
| 2350 | AAGGUC |
| 2351 | GACCAA |
| 2352 | CCUACA |
| 2353 | AUUGCC |
| 2354 | CAGACC |
| 2355 | ACAAGC |
| 2356 | CAGUCC |
| 2357 | GGACAC |
| 2358 | GCCAAC |
| 2359 | GCUGUA |
| 2360 | CUGGUA |
| 2361 | GGCCAC |
| 2362 | GAUGGC |
| 2363 | CAAUGC |
| 2364 | CACCUA |
| 2365 | GAUUGC |
| 2366 | AGUGCC |
| 2367 | CAUGAC |
| 2368 | GACCCA |
| 2369 | GAGGAC |
| 2370 | CAGCUG |
| 2371 | CCCUAA |
| 2372 | CAAUCC |
| 2373 | GUGAAC |
| 2374 | CCAACC |
| 2375 | ACUGAC |
| 2376 | CAGGUC |
| 2377 | AGCCUA |
| 2378 | GAAUCC |
| 2379 | AGGGUC |
| 2380 | GACAGC |
| 2381 | CUAGCA |
| 2382 | AGUAGG |
| 2383 | GAGGUA |
| 2384 | AUGGAC |
| 2385 | GCCCAA |
| 2386 | AUUGGG |
| 2387 | AUGGGC |
| 2388 | CAUAGG |
| 2389 | GUGGUA |
| 2390 | CCUAAG |
| 2391 | GGGCAC |
| 2392 | ACAACC |
| 2393 | CUGUAC |
| 2394 | CCACUA |
| 2395 | GGCACC |
| 2396 | AGCUAG |
| 2397 | GGGAAC |
| 2398 | CUGAUC |
| 2399 | GCAGAC |
| 2400 | AUGGUC |
| 2401 | AUUGGC |
| 2402 | GGGUCA |
| 2403 | CAACCC |
| 2404 | AGAUCC |
| 2405 | CUUGAC |
| 2406 | GGACUA |
| 2407 | CCUUAG |
| 2408 | GCAGUA |
| 2409 | CCCAUA |
| 2410 | GAGCUA |
| 2411 | GAUGAC |
| 2412 | GGUUGA |
| 2413 | GCUACA |
| 2414 | GCACCC |
| 2415 | GGCUUA |
| 2416 | GGGAUA |
| 2417 | GGUCAA |
| 2418 | AGGGUA |
| 2419 | AUGACC |
| 2420 | ACCUAG |
| 2421 | GUAGGA |
| 2422 | GCUAGA |
| 2423 | CAUAGC |
| 2424 | GUCCAA |
| 2425 | AGUUGC |
| 2426 | GGUACA |
| 2427 | GAGCAC |
| 2428 | GCCAUA |
| 2429 | CCAUAG |
| 2430 | CUUAGC |
| 2431 | GAUGCC |
| 2432 | GCUCUA |
| 2433 | AGGACC |
| 2434 | CCCUUA |
| 2435 | AUAGGC |
| 2436 | CACGCC |
| 2437 | GGCAUA |
| 2438 | GCAUCC |
| 2439 | GUGCUA |
| 2440 | CCAAUC |
| 2441 | GACAUC |
| 2442 | CAGGCG |
| 2443 | GUAGCA |
| 2444 | GGACCA |
| 2445 | GACUGC |
| 2446 | GGGUAA |
| 2447 | CUAGGC |
| 2448 | GCAAGC |
| 2449 | AGGCCU |
| 2450 | GGAGUA |
| 2451 | GACCAC |
| 2452 | GGAACC |
| 2453 | ACCCUA |
| 2454 | GAGUCC |
| 2455 | GCUGAC |
| 2456 | AGGUCC |
| 2457 | CCUACC |
| 2458 | GGUCAC |
| 2459 | GCCUUA |
| 2460 | GUACCA |
| 2461 | CUUACC |
| 2462 | GACUAC |
| 2463 | CUAUGC |
| 2464 | ACCUAC |
| 2465 | CUACAC |
| 2466 | CUAGAC |
| 2467 | AUACCC |
| 2468 | GACACC |
| 2469 | CACUAC |
| 2470 | AUAGGG |
| 2471 | GGGACC |
| 2472 | CUAAGC |
| 2473 | CCCUAG |
| 2474 | AUAGCC |
| 2475 | CAUACC |
| 2476 | GCCUAA |
| 2477 | CCUUAC |
| 2478 | GGUCCA |
| 2479 | CUAUCC |
| 2480 | CCUAGC |
| 2481 | ACUAGG |
| 2482 | GACAAC |
| 2483 | CAGUAC |
| 2484 | CUAGCC |
| 2485 | CCUAAC |
| 2486 | GGUGUA |
| 2487 | AGGUAC |
| 2488 | CGGGAG |
| 2489 | CACUAG |
| 2490 | GUCCAC |
| 2491 | GAUCCC |
| 2492 | AGGCGG |
| 2493 | GGACCC |
| 2494 | CUACCC |
| 2495 | CUCGGC |
| 2496 | CCUAUC |
| 2497 | CCAUAC |
| 2498 | GGCCUA |
| 2499 | CCCUAC |
| 2500 | CCAUGG |
| 2501 | GGGUUA |
| 2502 | ACUACC |
| 2503 | GUCCUA |
| 2504 | GCACUA |
| 2505 | GCUAAC |
| 2506 | GUACAC |
| 2507 | GACCUA |
| 2508 | AGUACC |
| 2509 | CUAACC |
| 2510 | GGUCUA |
| 2511 | GGAUAC |
| 2512 | GCUUAC |
| 2513 | GGGCUA |
| 2514 | GUCAAC |
| 2515 | CGCCUC |
| 2516 | CUAGUC |
| 2517 | GAUACC |
| 2518 | GCCCUA |
| 2519 | GGUAAC |
| 2520 | GAUAGC |
| 2521 | AGACGG |
| 2522 | GGCUAC |
| 2523 | CGUCUC |
| 2524 | ACUAGC |
| 2525 | GCAUAC |
| 2526 | GGUUAC |
| 2527 | GAGUAC |
| 2528 | CGAGAC |
| 2529 | GUAGAC |
| 2530 | AGGCCG |
| 2531 | GCCUAC |
| 2532 | GCUACC |
| 2533 | CGCCCA |
| 2534 | GCGUGA |
| 2535 | GAGCUC |
| 2536 | CCUAGG |
| 2537 | ACGCCU |
| 2538 | CCGAGG |
| 2539 | GGCGGA |
| 2540 | CGCCAC |
| 2541 | GGGUAC |
| 2542 | CGUGAG |
| 2543 | ACCACG |
| 2544 | GCAUGC |
| 2545 | CCACCG |
| 2546 | CCACGC |
| 2547 | CCGAGA |
| 2548 | CCCGAG |
| 2549 | CGGCUC |
| 2550 | CCGGGA |
| 2551 | UCGAGA |
| 2552 | GUGCAC |
| 2553 | CGAGGC |
| 2554 | CCUCCG |
| 2555 | GGCCGA |
| 2556 | CGGGCA |
| 2557 | ACCCGG |
| 2558 | AGCCGA |
| 2559 | CUCCGC |
| 2560 | AUCUCG |
| 2561 | CCCGUC |
| 2562 | CGUGCC |
| 2563 | AUCACG |
| 2564 | AGCCGG |
| 2565 | CACACG |
| 2566 | CACCGC |
| 2567 | UCCCGA |
| 2568 | AGAUCG |
| 2569 | CACCCG |
| 2570 | CGUUUC |
| 2571 | ACCUCG |
| 2572 | ACGUGG |
| 2573 | ACGGUG |
| 2574 | ACGGAG |
| 2575 | AAGCGA |
| 2576 | AAACGG |
| 2577 | AGCGAG |
| 2578 | AGGCGC |
| 2579 | CAAGCG |
| 2580 | CUCGAA |
| 2581 | ACUCGG |
| 2582 | ACCGUG |
| 2583 | AGAGCG |
| 2584 | AGCGAU |
| 2585 | CCGCCA |
| 2586 | CACGAG |
| 2587 | ACACGU |
| 2588 | AUGGCG |
| 2589 | AACGUG |
| 2590 | CCCACG |
| 2591 | CGUGAA |
| 2592 | ACACGG |
| 2593 | ACGAGG |
| 2594 | AACACG |
| 2595 | GCGCCA |
| 2596 | ACGCCA |
| 2597 | UCACGA |
| 2598 | GGAUCC |
| 2599 | GCGAGA |
| 2600 | AACCCG |
| 2601 | CAGCCG |
| 2602 | ACGUGA |
| 2603 | ACGCCC |
| 2604 | GAGCGA |
| 2605 | CCACGG |
| 2606 | GACGGA |
| 2607 | CGAUUC |
| 2608 | ACGGGA |
| 2609 | CGGCCA |
| 2610 | GGCGCA |
| 2611 | ACUCCG |
| 2612 | CACGCA |
| 2613 | GGGCGA |
| 2614 | AAUCGC |
| 2615 | AAAGCG |
| 2616 | CGGGAA |
| 2617 | CGGUGA |
| 2618 | CGUGGC |
| 2619 | CGCUGC |
| 2620 | ACGAAG |
| 2621 | ACGUGC |
| 2622 | CCAGCG |
| 2623 | AGUUCG |
| 2624 | CGCACC |
| 2625 | CGUGGA |
| 2626 | CGACAG |
| 2627 | CGGCAG |
| 2628 | CCGCAG |
| 2629 | CGAGUA |
| 2630 | GUUCGA |
| 2631 | AACGGG |
| 2632 | CGUGCA |
| 2633 | ACGCUG |
| 2634 | CAGACG |
| 2635 | CCAACG |
| 2636 | GCGGGA |
| 2637 | CAGCGG |
| 2638 | CGGAGC |
| 2639 | AGCGUG |
| 2640 | CGCUCC |
| 2641 | CUGCGC |
| 2642 | AUCGUG |
| 2643 | CCGUGC |
| 2644 | CCUGCG |
| 2645 | ACCGCA |
| 2646 | ACGGAA |
| 2647 | CACGGC |
| 2648 | AGCCCG |
| 2649 | CACGGG |
| 2650 | CACGGA |
| 2651 | CAACGA |
| 2652 | CACGUA |
| 2653 | AGCGGG |
| 2654 | CGGAAG |
| 2655 | CGAGAA |
| 2656 | CGCACA |
| 2657 | AGAACG |
| 2658 | CGCAGC |
| 2659 | CAAACG |
| 2660 | ACGCAG |
| 2661 | AAGACG |
| 2662 | GGUACC |
| 2663 | CCGGAG |
| 2664 | CGAGGA |
| 2665 | GAUCGC |
| 2666 | ACCCGC |
| 2667 | CACGUG |
| 2668 | GCUAGC |
| 2669 | ACACGC |
| 2670 | AAGGCG |
| 2671 | CGGCUA |
| 2672 | ACUGCG |
| 2673 | ACGUCU |
| 2674 | CAGCGA |
| 2675 | CACGUC |
| 2676 | ACGUUG |
| 2677 | GCGACA |
| 2678 | AUCCCG |
| 2679 | AGGGCG |
| 2680 | CCUCGC |
| 2681 | CGGAGA |
| 2682 | CGCCAG |
| 2683 | CCGUGA |
| 2684 | ACGAGA |
| 2685 | ACAGCG |
| 2686 | AGCACG |
| 2687 | AUCCGC |
| 2688 | UCGCCA |
| 2689 | CUCGCC |
| 2690 | CCUUCG |
| 2691 | CCACGA |
| 2692 | CAGCGC |
| 2693 | CCCUCG |
| 2694 | CCCGGA |
| 2695 | CGAGAG |
| 2696 | GGCGAC |
| 2697 | CCCGCA |
| 2698 | CACGAA |
| 2699 | AGACGA |
| 2700 | CGAAGA |
| 2701 | AGCCGC |
| 2702 | CGGAAA |
| 2703 | AUCGAG |
| 2704 | CGCAGA |
| 2705 | CGGAUC |
| 2706 | CCGCAC |
| 2707 | CGAAAC |
| 2708 | AUCGCA |
| 2709 | AAACCG |
| 2710 | GCACGC |
| 2711 | CAUUCG |
| 2712 | CGUGUC |
| 2713 | CGUUCA |
| 2714 | AACGGA |
| 2715 | GCACGA |
| 2716 | ACGCAC |
| 2717 | ACGAUG |
| 2718 | AGGACG |
| 2719 | AACGCA |
| 2720 | CGCUUC |
| 2721 | CGUCCC |
| 2722 | AAGCGG |
| 2723 | AGGCGA |
| 2724 | ACACGA |
| 2725 | ACGGCA |
| 2726 | CCUCGA |
| 2727 | ACGUCA |
| 2728 | CCGCUC |
| 2729 | CAUGCG |
| 2730 | AAGCCG |
| 2731 | AACGAG |
| 2732 | CCGGCA |
| 2733 | AAGCGC |
| 2734 | GAGCGC |
| 2735 | AAACGC |
| 2736 | ACGUUC |
| 2737 | ACCGAG |
| 2738 | CGCUGA |
| 2739 | CCGAGC |
| 2740 | CCGUCC |
| 2741 | CGCUCA |
| 2742 | GCGAAA |
| 2743 | GCGGCA |
| 2744 | CCGAAG |
| 2745 | AACGCU |
| 2746 | ACACCG |
| 2747 | AGCGGC |
| 2748 | CGAAAG |
| 2749 | CUCGGA |
| 2750 | CGUGUA |
| 2751 | AGCGAA |
| 2752 | CCAUCG |
| 2753 | CCCGAC |
| 2754 | AGCGCC |
| 2755 | CGUUCC |
| 2756 | AUGACG |
| 2757 | CGAGCC |
| 2758 | CACUCG |
| 2759 | CUGCGA |
| 2760 | CUCGUC |
| 2761 | AAGCGU |
| 2762 | ACAACG |
| 2763 | GCCGCA |
| 2764 | AACUCG |
| 2765 | CCGAAA |
| 2766 | CGUCCA |
| 2767 | CUCCGA |
| 2768 | ACGAUC |
| 2769 | ACGUCC |
| 2770 | ACCGGG |
| 2771 | CACCGG |
| 2772 | CGGCAC |
| 2773 | AACGUC |
| 2774 | AGACGC |
| 2775 | CCGGAA |
| 2776 | CGUCAG |
| 2777 | ACUUCG |
| 2778 | ACGGCU |
| 2779 | CACCGA |
| 2780 | AGCGGA |
| 2781 | CGUGAC |
| 2782 | CGCAAA |
| 2783 | AGGUCG |
| 2784 | CGAUGA |
| 2785 | ACGUAG |
| 2786 | CGUCAC |
| 2787 | CCCGAA |
| 2788 | GAUCGA |
| 2789 | CGAGCA |
| 2790 | CGGUGC |
| 2791 | AGCGCA |
| 2792 | AUGCGG |
| 2793 | CAUACG |
| 2794 | CCGUCA |
| 2795 | AGACCG |
| 2796 | UCGUCA |
| 2797 | ACGCAU |
| 2798 | ACGGGC |
| 2799 | GGCGAA |
| 2800 | GAACGA |
| 2801 | AGCUCG |
| 2802 | ACGACA |
| 2803 | CCGCAA |
| 2804 | ACGGCC |
| 2805 | GCGCAC |
| 2806 | ACAUCG |
| 2807 | GCCGGA |
| 2808 | CAUCGC |
| 2809 | UCCGCA |
| 2810 | CGGGAC |
| 2811 | CAUCGA |
| 2812 | ACCGCC |
| 2813 | AUUCCG |
| 2814 | CAUCCG |
| 2815 | ACCCGU |
| 2816 | CUUCGA |
| 2817 | UCGGAA |
| 2818 | ACGCAA |
| 2819 | AUUGCG |
| 2820 | ACGAGU |
| 2821 | GAACGC |
| 2822 | ACCCGA |
| 2823 | AUUCGG |
| 2824 | UCGGCA |
| 2825 | AAUGCG |
| 2826 | CCGACC |
| 2827 | CCGUUC |
| 2828 | AUGCCG |
| 2829 | AUCGCC |
| 2830 | ACCGAA |
| 2831 | AACCGA |
| 2832 | UCCGAA |
| 2833 | ACCGCU |
| 2834 | CGUAAG |
| 2835 | ACUCGA |
| 2836 | CGUUGA |
| 2837 | AACCGU |
| 2838 | GCGUCC |
| 2839 | CUCGCA |
| 2840 | GCGGAA |
| 2841 | CGGCAA |
| 2842 | CGAAGC |
| 2843 | GCCCGA |
| 2844 | ACGCUC |
| 2845 | AGCGUC |
| 2846 | GUGCGA |
| 2847 | CGGACA |
| 2848 | AACGGU |
| 2849 | CGAACA |
| 2850 | ACGGAU |
| 2851 | GACGCA |
| 2852 | CUUCGC |
| 2853 | AGUGCG |
| 2854 | CCGUAA |
| 2855 | CGGGUC |
| 2856 | CGUAGA |
| 2857 | CGUUGC |
| 2858 | CGCCAA |
| 2859 | AGCGCU |
| 2860 | GACGAA |
| 2861 | GACGCC |
| 2862 | CGAGUC |
| 2863 | ACCGUA |
| 2864 | CGAUCC |
| 2865 | GCCGAA |
| 2866 | AUACGG |
| 2867 | CGAUCA |
| 2868 | CGCAAG |
| 2869 | GCGAGC |
| 2870 | AAGUCG |
| 2871 | CACGAC |
| 2872 | CCGACA |
| 2873 | AACGCC |
| 2874 | ACGGUA |
| 2875 | UACGCA |
| 2876 | AGUCGG |
| 2877 | ACUACG |
| 2878 | CGGUUC |
| 2879 | AUGCGC |
| 2880 | ACCGCG |
| 2881 | AAUCCG |
| 2882 | GACGGC |
| 2883 | ACCGUC |
| 2884 | CAGUCG |
| 2885 | CGCCUA |
| 2886 | CGUCUA |
| 2887 | ACGUAC |
| 2888 | CGCAUC |
| 2889 | CGGUCA |
| 2890 | CGUACA |
| 2891 | CAACGG |
| 2892 | UGCGAA |
| 2893 | CGAUGC |
| 2894 | AUGUCG |
| 2895 | CGACUC |
| 2896 | CCUACG |
| 2897 | GGACGA |
| 2898 | ACCGGC |
| 2899 | CGACAA |
| 2900 | UACGGA |
| 2901 | CCGGAC |
| 2902 | AACGGC |
| 2903 | CGUCAA |
| 2904 | CGUAUC |
| 2905 | AACCGC |
| 2906 | CAUCGG |
| 2907 | CGUUAG |
| 2908 | AUGCGA |
| 2909 | GCGCAA |
| 2910 | AAUCGG |
| 2911 | ACGAGC |
| 2912 | AUCGUC |
| 2913 | CUCGAC |
| 2914 | AGUCCG |
| 2915 | UCGCAA |
| 2916 | GCGUCA |
| 2917 | ACGACU |
| 2918 | AACCGG |
| 2919 | CGGUCC |
| 2920 | CGGGUA |
| 2921 | ACGGAC |
| 2922 | CGACCC |
| 2923 | UCGACA |
| 2924 | CGACAC |
| 2925 | ACGAAC |
| 2926 | GCUCGA |
| 2927 | CUACGA |
| 2928 | CCGGUC |
| 2929 | CGAAUC |
| 2930 | CAACGC |
| 2931 | AGCGAC |
| 2932 | CCGUAG |
| 2933 | ACUCGC |
| 2934 | AUCGGG |
| 2935 | CGGUAA |
| 2936 | AACGAC |
| 2937 | AGUCGA |
| 2938 | AUCCGG |
| 2939 | CCCGUA |
| 2940 | ACGCUA |
| 2941 | CAACCG |
| 2942 | ACGGUC |
| 2943 | GACGUA |
| 2944 | AUAGCG |
| 2945 | CGACCA |
| 2946 | CAAUCG |
| 2947 | CGGAAC |
| 2948 | CGGUAG |
| 2949 | CUCGUA |
| 2950 | AUCCGA |
| 2951 | GACCGA |
| 2952 | ACCGGA |
| 2953 | CGCAUA |
| 2954 | AUACGC |
| 2955 | AGCGUA |
| 2956 | GACCGC |
| 2957 | CGCUUA |
| 2958 | CGCUAA |
| 2959 | CCGUUA |
| 2960 | AUUCGC |
| 2961 | CGUAGC |
| 2962 | UAGCGA |
| 2963 | CGCAAC |
| 2964 | CGGACC |
| 2965 | AUCGGA |
| 2966 | CGUAAC |
| 2967 | CGAUAG |
| 2968 | GCCGAC |
| 2969 | CUCGAG |
| 2970 | ACCGAU |
| 2971 | AUACCG |
| 2972 | CUACGC |
| 2973 | AGUACG |
| 2974 | GUCGAA |
| 2975 | GGCGUA |
| 2976 | GCGGAC |
| 2977 | UACCGA |
| 2978 | CCGCUA |
| 2979 | GGUCGA |
| 2980 | GCGAUA |
| 2981 | ACGACC |
| 2982 | AGUCGC |
| 2983 | GCGCGA |
| 2984 | CGUUAC |
| 2985 | AUCGGC |
| 2986 | CGGAUA |
| 2987 | GCGUAA |
| 2988 | CGAACC |
| 2989 | GCGUUA |
| 2990 | GACGAC |
| 2991 | CGCUAC |
| 2992 | GCGACC |
| 2993 | CGGUUA |
| 2994 | CCGAAC |
| 2995 | GUCGGA |
| 2996 | GUCGCA |
| 2997 | GCCGUA |
| 2998 | CCGGUA |
| 2999 | CGUACC |
| 3000 | ACCGAC |
| 3001 | CCGAUC |
| 3002 | AUCGCG |
| 3003 | CGCUAG |
| 3004 | GUCCGA |
| 3005 | UCCGGA |
| 3006 | CCGAUA |
| 3007 | AUCGAC |
| 3008 | GCGGUA |
| 3009 | GCGCUA |
| 3010 | CACGCG |
| 3011 | GUACGA |
| 3012 | UGCGCA |
| 3013 | GUCGUA |
| 3014 | CGACUA |
| 3015 | CCGUAC |
| 3016 | CGAUAC |
| 3017 | CGGUAC |
| 3018 | CGCACG |
| 3019 | GCGAAC |
| 3020 | CGCGCA |
| 3021 | AGCGCG |
| 3022 | ACGCGG |
| 3023 | ACGGCG |
| 3024 | GACGUC |
| 3025 | CGAGCG |
| 3026 | CGGACG |
| 3027 | ACGCCG |
| 3028 | ACGCGC |
| 3029 | GCGUAC |
| 3030 | CGCGAG |
| 3031 | CGCGGA |
| 3032 | CGCGUC |
| 3033 | CGGCGA |
| 3034 | CCGCGA |
| 3035 | CGCCGA |
| 3036 | ACCGGU |
| 3037 | CCGACG |
| 3038 | CCGUCG |
| 3039 | CGUCGC |
| 3040 | CGACGC |
| 3041 | ACGACG |
| 3042 | AACGCG |
| 3043 | ACGUCG |
| 3044 | CGCGAC |
| 3045 | CGAACG |
| 3046 | CGACCG |
| 3047 | ACGCGA |
| 3048 | CGCGAA |
| 3049 | CGACGA |
| 3050 | GUCGAC |
| 3051 | CGUCGA |
| 3052 | CGCGUA |
| 3053 | ACGCGU |
| 3054 | UCGCGA |
| 3055 | CGAUCG |
| 3056 | CGUACG |

TABLE 3
Exemplary sequences of binding segments
of the donor DNAs (5-nucleotide long)

| SEQ | |
|---|---|
| ID | |
| NO. | Sequence |
| 3057 | CCCAG |
| 3058 | TCCCA |
| 3059 | CAGAG |
| 3060 | CCAGG |
| 3061 | AGGAG |
| 3062 | CAGGA |
| 3063 | CCTCC |
| 3064 | CTCAG |
| 3065 | CCAGC |
| 3066 | AGGCA |
| 3067 | CTGCA |
| 3068 | CAGCC |
| 3069 | CACAG |
| 3070 | CAGCA |
| 3071 | CACTG |
| 3072 | CTCCA |
| 3073 | CTCCC |
| 3074 | CAGGC |
| 3075 | AGCCT |
| 3076 | CACAC |
| 3077 | CCTCA |
| 3078 | AGCCA |
| 3079 | CTGGA |
| 3080 | GGAGA |
| 3081 | CTTCC |
| 3082 | AGCTG |
| 3083 | CACCA |
| 3084 | GAGGC |
| 3085 | AGAGG |
| 3086 | AGCAG |
| 3087 | CTCTC |
| 3088 | CTGCC |
| 3089 | ACAGG |
| 3090 | AGGGA |
| 3091 | CCTGA |
| 3092 | CCACA |
| 3093 | CCACC |
| 3094 | GAGGA |
| 3095 | GGGAA |
| 3096 | GCAGA |
| 3097 | CCAGA |
| 3098 | AGGTG |
| 3099 | CTCAC |
| 3100 | TGCCA |
| 3101 | CAGGG |
| 3102 | CCTTC |
| 3103 | AGTGG |
| 3104 | CCCAA |
| 3105 | GCTGA |
| 3106 | CATGG |
| 3107 | CCTGC |
| 3108 | ACCTG |
| 3109 | CTGGC |
| 3110 | GAGAC |
| 3111 | CTGTC |
| 3112 | CCAAG |
| 3113 | GCTCA |
| 3114 | ACCCA |
| 3115 | ACTGC |
| 3116 | ACCTC |
| 3117 | GGCCA |
| 3118 | CCCAC |
| 3119 | CCATC |
| 3120 | CAAGG |
| 3121 | GCCCA |
| 3122 | ACTCC |
| 3123 | AAGGG |
| 3124 | GGTGA |
| 3125 | CACTC |
| 3126 | AGAGC |
| 3127 | GTGGA |
| 3128 | ATGGG |
| 3129 | GCACA |
| 3130 | CCCTC |
| 3131 | ACCAG |
| 3132 | ACCAC |
| 3133 | GGGCA |
| 3134 | GCCAC |
| 3135 | AGGCC |
| 3136 | GAGCA |
| 3137 | ACTGG |
| 3138 | CATGC |
| 3139 | ACAGC |
| 3140 | GTGCA |
| 3141 | GCCAA |
| 3142 | CACCC |
| 3143 | AGCAC |
| 3144 | ATCCC |
| 3145 | AAGGC |
| 3146 | AGCTC |
| 3147 | GGCAA |
| 3148 | GAGCC |
| 3149 | AGTGC |
| 3150 | ATGCC |
| 3151 | GCAGC |
| 3152 | GAAGC |
| 3153 | ATGGC |
| 3154 | AAGCC |
| 3155 | CATCC |
| 3156 | GGACA |
| 3157 | CAGAC |
| 3158 | CTTGC |
| 3159 | CTGAC |
| 3160 | AACCC |
| 3161 | AGACC |
| 3162 | AGCCC |
| 3163 | ACCCT |
| 3164 | ACACC |
| 3165 | CAAGC |
| 3166 | CCAAC |
| 3167 | GACCA |
| 3168 | CAGTC |
| 3169 | AGGGC |
| 3170 | AGGAC |
| 3171 | GGTCA |
| 3172 | GCTCC |
| 3173 | AGGTC |
| 3174 | GGCAC |
| 3175 | CAACC |
| 3176 | AGTCC |
| 3177 | GACAC |
| 3178 | CCTAG |
| 3179 | GGAAC |
| 3180 | GTCCA |
| 3181 | GTCAC |
| 3182 | GCAAC |
| 3183 | GACTC |
| 3184 | GAACC |
| 3185 | GGGAC |
| 3186 | GCACC |
| 3187 | GGCTA |
| 3188 | GATGC |
| 3189 | CCCTA |
| 3190 | CTACC |
| 3191 | GATCC |
| 3192 | GCTAC |
| 3193 | GCCTA |
| 3194 | CCTAC |
| 3195 | GACCC |
| 3196 | GGGTA |
| 3197 | CTAGC |
| 3198 | GGACC |
| 3199 | GGTAC |
| 3200 | AGGCG |
| 3201 | CCGAG |
| 3202 | CACGC |
| 3203 | CGAGA |
| 3204 | CGGGA |
| 3205 | CGTGA |
| 3206 | CCACG |
| 3207 | ACGTG |
| 3208 | CCTCG |
| 3209 | AGACG |
| 3210 | CGCCA |
| 3211 | CGGAG |
| 3212 | AGCCG |
| 3213 | ACGCC |
| 3214 | CACGG |
| 3215 | CACCG |
| 3216 | ACACG |
| 3217 | ACGGG |
| 3218 | GCCGA |
| 3219 | AGCGA |
| 3220 | CAGCG |
| 3221 | CCGTC |
| 3222 | CGTGC |
| 3223 | ACCCG |
| 3224 | CGCAG |
| 3225 | AAGCG |
| 3226 | CACGA |
| 3227 | CCCGA |
| 3228 | CTCGA |
| 3229 | ACGGA |
| 3230 | GCGGA |
| 3231 | CGCTC |
| 3232 | ACGAG |
| 3233 | CCGCA |
| 3234 | CTCGC |
| 3235 | ATCGC |
| 3236 | ACGCA |
| 3237 | ACTCG |
| 3238 | CGAAG |
| 3239 | GGCGA |
| 3240 | ACCGT |
| 3241 | AGCGG |
| 3242 | AACGG |
| 3243 | CGGCA |
| 3244 | CAACG |
| 3245 | CGGAA |
| 3246 | CGCAC |
| 3247 | ACGCT |
| 3248 | AGCGC |
| 3249 | ACCGC |
| 3250 | GCGCA |
| 3251 | CGATC |
| 3252 | CGTTC |
| 3253 | CGTCA |
| 3254 | CCGGA |
| 3255 | CGACA |
| 3256 | CATCG |
| 3257 | CGTCC |
| 3258 | ACGTC |
| 3259 | CCGAA |
| 3260 | ATCCG |
| 3261 | ACGGC |
| 3262 | ATGCG |
| 3263 | TCGCA |
| 3264 | CGAGC |
| 3265 | CGCAA |
| 3266 | AACGC |
| 3267 | AACCG |
| 3268 | CGAAC |
| 3269 | GACGA |
| 3270 | ACCGA |
| 3271 | TCCGA |
| 3272 | CGTAG |
| 3273 | GCGAA |
| 3274 | ACCGG |
| 3275 | GACGC |
| 3276 | AGTCG |
| 3277 | CCGTA |
| 3278 | CCGAC |
| 3279 | GCGAC |
| 3280 | CGGTC |
| 3281 | CGACC |
| 3282 | CGGAC |
| 3283 | ACGAC |
| 3284 | ATCGG |
| 3285 | CGGTA |
| 3286 | CGCTA |
| 3287 | GCGTA |
| 3288 | GTCGA |
| 3289 | CGTAC |
| 3290 | CGCGA |
| 3291 | ACGCG |
| 3292 | CGACG |
| 3293 | CCCAG |
| 3294 | TCCCA |
| 3295 | CAGAG |
| 3296 | CCAGG |
| 3297 | AGGAG |
| 3298 | CAGGA |
| 3299 | CCTCC |
| 3300 | CTCAG |
| 3301 | CCAGC |
| 3302 | AGGCA |
| 3303 | CTGCA |
| 3304 | CAGCC |
| 3305 | CACAG |
| 3306 | CAGCA |
| 3307 | CACTG |
| 3308 | CTCCA |
| 3309 | CTCCC |
| 3310 | CAGGC |
| 3311 | AGCCT |
| 3312 | CACAC |
| 3313 | CCTCA |
| 3314 | AGCCA |
| 3315 | CTGGA |
| 3316 | GGAGA |
| 3317 | CTTCC |
| 3318 | AGCTG |
| 3319 | CACCA |
| 3320 | GAGGC |
| 3321 | AGAGG |
| 3322 | AGCAG |
| 3323 | CTCTC |
| 3324 | CTGCC |
| 3325 | ACAGG |
| 3326 | AGGGA |
| 3327 | CCTGA |
| 3328 | CCACA |
| 3329 | CCACC |
| 3330 | GAGGA |
| 3331 | GGGAA |
| 3332 | GCAGA |
| 3333 | CCAGA |
| 3334 | AGGTG |
| 3335 | CTCAC |
| 3336 | TGCCA |
| 3337 | CAGGG |
| 3338 | CCTTC |
| 3339 | AGTGG |
| 3340 | CCCAA |
| 3341 | GCTGA |
| 3342 | CATGG |
| 3343 | CCTGC |
| 3344 | ACCTG |
| 3345 | CTGGC |
| 3346 | GAGAC |
| 3347 | CTGTC |
| 3348 | CCAAG |
| 3349 | GCTCA |
| 3350 | ACCCA |
| 3351 | ACTGC |
| 3352 | ACCTC |
| 3353 | GGCCA |
| 3354 | CCCAC |
| 3355 | CCATC |
| 3356 | CAAGG |
| 3357 | GCCCA |
| 3358 | ACTCC |
| 3359 | AAGGG |
| 3360 | GGTGA |
| 3361 | CACTC |
| 3362 | AGAGC |
| 3363 | GTGGA |
| 3364 | ATGGG |
| 3365 | GCACA |
| 3366 | CCCTC |
| 3367 | ACCAG |
| 3368 | ACCAC |
| 3369 | GGGCA |
| 3370 | GCCAC |
| 3371 | AGGCC |
| 3372 | GAGCA |
| 3373 | ACTGG |
| 3374 | CATGC |
| 3375 | ACAGC |
| 3376 | GTGCA |
| 3377 | GCCAA |
| 3378 | CACCC |
| 3379 | AGCAC |
| 3380 | ATCCC |
| 3381 | AAGGC |
| 3382 | AGCTC |
| 3383 | GGCAA |
| 3384 | GAGCC |
| 3385 | AGTGC |
| 3386 | ATGCC |
| 3387 | GCAGC |
| 3388 | GAAGC |
| 3389 | ATGGC |
| 3390 | AAGCC |
| 3391 | CATCC |
| 3392 | GGACA |
| 3393 | CAGAC |
| 3394 | CTTGC |
| 3395 | CTGAC |
| 3396 | AACCC |
| 3397 | AGACC |
| 3398 | AGCCC |
| 3399 | ACCCT |
| 3400 | ACACC |
| 3401 | CAAGC |
| 3402 | CCAAC |
| 3403 | GACCA |
| 3404 | CAGTC |
| 3405 | AGGGC |
| 3406 | AGGAC |
| 3407 | GGTCA |
| 3408 | GCTCC |
| 3409 | AGGTC |
| 3410 | GGCAC |
| 3411 | CAACC |
| 3412 | AGTCC |
| 3413 | GACAC |
| 3414 | CCTAG |
| 3415 | GGAAC |
| 3416 | GTCCA |
| 3417 | GTCAC |
| 3418 | GCAAC |
| 3419 | GACTC |
| 3420 | GAACC |
| 3421 | GGGAC |
| 3422 | GCACC |
| 3423 | GGCTA |
| 3424 | GATGC |
| 3425 | CCCTA |
| 3426 | CTACC |
| 3427 | GATCC |
| 3428 | GCTAC |
| 3429 | GCCTA |
| 3430 | CCTAC |
| 3431 | GACCC |
| 3432 | GGGTA |
| 3433 | CTAGC |
| 3434 | GGACC |
| 3435 | GGTAC |
| 3436 | AGGCG |
| 3437 | CCGAG |
| 3438 | CACGC |
| 3439 | CGAGA |
| 3440 | CGGGA |
| 3441 | CGTGA |
| 3442 | CCACG |
| 3443 | ACGTG |
| 3444 | CCTCG |
| 3445 | AGACG |
| 3446 | CGCCA |
| 3447 | CGGAG |
| 3448 | AGCCG |
| 3449 | ACGCC |
| 3450 | CACGG |
| 3451 | CACCG |
| 3452 | ACACG |
| 3453 | ACGGG |
| 3454 | GCCGA |
| 3455 | AGCGA |
| 3456 | CAGCG |
| 3457 | CCGTC |
| 3458 | CGTGC |
| 3459 | ACCCG |
| 3460 | CGCAG |
| 3461 | AAGCG |
| 3462 | CACGA |
| 3463 | CCCGA |
| 3464 | CTCGA |
| 3465 | ACGGA |
| 3466 | GCGGA |
| 3467 | CGCTC |
| 3468 | ACGAG |
| 3469 | CCGCA |
| 3470 | CTCGC |
| 3471 | ATCGC |
| 3472 | ACGCA |
| 3473 | ACTCG |
| 3474 | CGAAG |
| 3475 | GGCGA |
| 3476 | ACCGT |
| 3477 | AGCGG |
| 3478 | AACGG |
| 3479 | CGGCA |
| 3480 | CAACG |
| 3481 | CGGAA |
| 3482 | CGCAC |
| 3483 | ACGCT |
| 3484 | AGCGC |
| 3485 | ACCGC |
| 3486 | GCGCA |
| 3487 | CGATC |
| 3488 | CGTTC |
| 3489 | CGTCA |
| 3490 | CCGGA |
| 3491 | CGACA |
| 3492 | CATCG |
| 3493 | CGTCC |
| 3494 | ACGTC |
| 3495 | CCGAA |
| 3496 | ATCCG |
| 3497 | ACGGC |
| 3498 | ATGCG |
| 3499 | TCGCA |
| 3500 | CGAGC |
| 3501 | CGCAA |
| 3502 | AACGC |
| 3503 | AACCG |
| 3504 | CGAAC |
| 3505 | GACGA |
| 3506 | ACCGA |
| 3507 | TCCGA |
| 3508 | CGTAG |
| 3509 | GCGAA |
| 3510 | ACCGG |
| 3511 | GACGC |
| 3512 | AGTCG |
| 3513 | CCGTA |
| 3514 | CCGAC |
| 3515 | GCGAC |
| 3516 | CGGTC |
| 3517 | CGACC |
| 3518 | CGGAC |
| 3519 | ACGAC |
| 3520 | ATCGG |
| 3521 | CGGTA |
| 3522 | CGCTA |
| 3523 | GCGTA |
| 3524 | GTCGA |
| 3525 | CGTAC |
| 3526 | CGCGA |
| 3527 | ACGCG |
| 3528 | CGACG |

TABLE 4
Exemplary sequences of binding segments
of the donor DNAs (6-nucleotide long)

| SEQ | |
|---|---|
| ID | |
| NO. | Sequence |
| 3529 | AGGCTG |
| 3530 | CACACA |
| 3531 | CCTCCC |
| 3532 | CAGGAG |
| 3533 | CTGGGA |
| 3534 | CCCAGC |
| 3535 | ACACAC |
| 3536 | CTCCCA |
| 3537 | CCCAGG |
| 3538 | AGGCAG |
| 3539 | ACAGAG |
| 3540 | AGAGAG |
| 3541 | CCAGGC |
| 3542 | GCCTCC |
| 3543 | CCAGCC |
| 3544 | AGGAAG |
| 3545 | AGGAGA |
| 3546 | CCTCAG |
| 3547 | GAGAGA |
| 3548 | AGCCTG |
| 3549 | GGAGAA |
| 3550 | CAGAGA |
| 3551 | ACTGCA |
| 3552 | CTCAGC |
| 3553 | AGACAG |
| 3554 | CACTGC |
| 3555 | AGGAGG |
| 3556 | CCCAAA |
| 3557 | CTGAGA |
| 3558 | CTCCAG |
| 3559 | CAGGCA |
| 3560 | AGCTGG |
| 3561 | AGTGAG |
| 3562 | CACAGA |
| 3563 | TCAGGA |
| 3564 | CAGGAA |
| 3565 | CCACCA |
| 3566 | CCTGCC |
| 3567 | CAGTGA |
| 3568 | CTGGGC |
| 3569 | CCTGGC |
| 3570 | TCCCAA |
| 3571 | CCAGGA |
| 3572 | CAGAAG |
| 3573 | CCAAAG |
| 3574 | CCAGCA |
| 3575 | GAGGCA |
| 3576 | ATCCCA |
| 3577 | AGCCTC |
| 3578 | CAGTGG |
| 3579 | GGCTCA |
| 3580 | GACAGA |
| 3581 | TGGGAA |
| 3582 | GGAAGA |
| 3583 | AGGGAA |
| 3584 | GGGAAA |
| 3585 | CTGGAA |
| 3586 | TCTCCA |
| 3587 | GAGGAA |
| 3588 | CACCTG |
| 3589 | GAAGGA |
| 3590 | ACACAG |
| 3591 | GGCTGA |
| 3592 | GCCTCA |
| 3593 | AGAGGA |
| 3594 | GAGACA |
| 3595 | GCTGGA |
| 3596 | CTTTCC |
| 3597 | CTGCAA |
| 3598 | CTTCTC |
| 3599 | CCTGAG |
| 3600 | CCTGTA |
| 3601 | CAGAGG |
| 3602 | AGATGG |
| 3603 | ATGGTG |
| 3604 | CACCAC |
| 3605 | ACTCCA |
| 3606 | ACCCAG |
| 3607 | TGCCCA |
| 3608 | AGAAGG |
| 3609 | CTTCCA |
| 3610 | AGCCAG |
| 3611 | CCTTCC |
| 3612 | GGAGGA |
| 3613 | AGGGAG |
| 3614 | CTGTGA |
| 3615 | ACTCTG |
| 3616 | CAGCAG |
| 3617 | CCAGAA |
| 3618 | GGCAGA |
| 3619 | CTCAGA |
| 3620 | TCTGCA |
| 3621 | GTCTCA |
| 3622 | CTCTGC |
| 3623 | AAGGAG |
| 3624 | GAGCCA |
| 3625 | AGGTGG |
| 3626 | CATCTC |
| 3627 | AGCAGA |
| 3628 | ACAGGC |
| 3629 | AAGCAG |
| 3630 | CTTCCC |
| 3631 | CCTTTC |
| 3632 | AATCCC |
| 3633 | AGAGCA |
| 3634 | GCAGGA |
| 3635 | GCAGAA |
| 3636 | TCCTCA |
| 3637 | CCCACC |
| 3638 | CTCTTC |
| 3639 | CCTCCA |
| 3640 | CAGCCA |
| 3641 | AAGGGA |
| 3642 | CTCACA |
| 3643 | AAGGCA |
| 3644 | AGAGAC |
| 3645 | CAGGGA |
| 3646 | CAGCAC |
| 3647 | ACAGCA |
| 3648 | ACATGG |
| 3649 | CTGGCC |
| 3650 | TCACCA |
| 3651 | ACAGTG |
| 3652 | AAGAGG |
| 3653 | CACCCA |
| 3654 | CTCTGA |
| 3655 | GGGAGA |
| 3656 | AGACCA |
| 3657 | AGCCAC |
| 3658 | AGCACT |
| 3659 | ACCACA |
| 3660 | CACATG |
| 3661 | CTCCTC |
| 3662 | CAGAGC |
| 3663 | CAGCAA |
| 3664 | AAGCCA |
| 3665 | CCCAGA |
| 3666 | CCACAG |
| 3667 | CTGAAG |
| 3668 | CAGCTA |
| 3669 | GCTCAC |
| 3670 | CTCTCC |
| 3671 | AGGCAT |
| 3672 | ACCTCA |
| 3673 | CCCTCC |
| 3674 | CCTGGA |
| 3675 | ACCATG |
| 3676 | CAGACA |
| 3677 | CCTCTC |
| 3678 | TCCAGA |
| 3679 | AGCTGA |
| 3680 | CAAAGG |
| 3681 | ATGGAG |
| 3682 | AGAGTG |
| 3683 | GTGAGA |
| 3684 | AGGCCA |
| 3685 | CTGCAC |
| 3686 | ACTCCT |
| 3687 | AAACCC |
| 3688 | AAAGGG |
| 3689 | TCAGCA |
| 3690 | CCAGAG |
| 3691 | CCAACA |
| 3692 | AGCCAA |
| 3693 | GAGGGA |
| 3694 | GAAGCA |
| 3695 | CAGATG |
| 3696 | GAGGCC |
| 3697 | ACTGTG |
| 3698 | CATGCC |
| 3699 | CCTGAA |
| 3700 | CCAAGA |
| 3701 | CTCTCA |
| 3702 | TGCACA |
| 3703 | ATGCTG |
| 3704 | CCTCAA |
| 3705 | AACTCC |
| 3706 | CTGCCA |
| 3707 | CACTCC |
| 3708 | AGGTGA |
| 3709 | CACAGG |
| 3710 | AGCAAG |
| 3711 | AAGCTG |
| 3712 | ACAGGA |
| 3713 | CACCTC |
| 3714 | AGCAGG |
| 3715 | AGGTCA |
| 3716 | GCCACC |
| 3717 | AGTGCA |
| 3718 | AGAAGC |
| 3719 | AGGCAA |
| 3720 | ACACTG |
| 3721 | CCATTC |
| 3722 | CACAGC |
| 3723 | CTGCCC |
| 3724 | CTCAAG |
| 3725 | ACTCAG |
| 3726 | CTGCTC |
| 3727 | TCCACA |
| 3728 | ATGGGA |
| 3729 | CAAGTG |
| 3730 | GGAAAC |
| 3731 | AGGACA |
| 3732 | CAGCTC |
| 3733 | CAAGGA |
| 3734 | GAAACC |
| 3735 | ATGGCA |
| 3736 | CCTTCA |
| 3737 | AGCTCA |
| 3738 | CTGTCA |
| 3739 | CATGAG |
| 3740 | CACACC |
| 3741 | CTCCAA |
| 3742 | GACACA |
| 3743 | ACCTGG |
| 3744 | CACTGA |
| 3745 | AAGTGG |
| 3746 | CAAGAG |
| 3747 | ATGTGG |
| 3748 | AGGATG |
| 3749 | ACCTCC |
| 3750 | GATGGA |
| 3751 | AGAGGG |
| 3752 | CCACCC |
| 3753 | CCAGGG |
| 3754 | CATGGA |
| 3755 | ACCTCT |
| 3756 | GTGGGA |
| 3757 | GTGGCA |
| 3758 | CATTCC |
| 3759 | ACTGAG |
| 3760 | AGTGGC |
| 3761 | AGCACA |
| 3762 | CCATCA |
| 3763 | ATGCAG |
| 3764 | AAGTGC |
| 3765 | CAAAGC |
| 3766 | CTTTGC |
| 3767 | GGTGGA |
| 3768 | GTGGAA |
| 3769 | AGAGCT |
| 3770 | CAACAG |
| 3771 | CCCACA |
| 3772 | GGTGAA |
| 3773 | ACTGCT |
| 3774 | AGTCTC |
| 3775 | GGCAAA |
| 3776 | CCTGCA |
| 3777 | AGGTTG |
| 3778 | GCTGCA |
| 3779 | TGAGCA |
| 3780 | AGTGGA |
| 3781 | AGACTG |
| 3782 | AGCCCA |
| 3783 | AACCTC |
| 3784 | ATGCCA |
| 3785 | CAAGCA |
| 3786 | AACCCA |
| 3787 | GCAGCA |
| 3788 | ACAGGG |
| 3789 | CTCCAC |
| 3790 | GAGTGA |
| 3791 | AGCAGC |
| 3792 | CCCAAG |
| 3793 | GCTGAA |
| 3794 | CTTGGC |
| 3795 | AATGGG |
| 3796 | CCTGAC |
| 3797 | CCACAA |
| 3798 | GGCCAA |
| 3799 | ACACCA |
| 3800 | CTGGCA |
| 3801 | GTGTGA |
| 3802 | TGGCAA |
| 3803 | AGCCAT |
| 3804 | CCAAGG |
| 3805 | ACCAGC |
| 3806 | ATCCTG |
| 3807 | CATCAC |
| 3808 | CCCTGC |
| 3809 | ATGAGG |
| 3810 | CAGCCC |
| 3811 | CATGCA |
| 3812 | GGCACA |
| 3813 | AGAGGC |
| 3814 | CCCTTC |
| 3815 | AGTGTG |
| 3816 | AACCTG |
| 3817 | AGACAC |
| 3818 | AAAGGC |
| 3819 | CCACAC |
| 3820 | GCAAGA |
| 3821 | CCCATC |
| 3822 | CTGTGC |
| 3823 | ACCCTG |
| 3824 | AAAGCC |
| 3825 | CTCTAC |
| 3826 | ACAGGT |
| 3827 | ACTGGA |
| 3828 | ACAGCT |
| 3829 | CATCCA |
| 3830 | ACTGGG |
| 3831 | CACTCA |
| 3832 | ATCCAG |
| 3833 | CTGTTC |
| 3834 | ATCTCC |
| 3835 | CCTGTC |
| 3836 | CTACAG |
| 3837 | AGTGGG |
| 3838 | GGTTCA |
| 3839 | ACTTCC |
| 3840 | AGCATG |
| 3841 | AGGGCA |
| 3842 | TAGCCA |
| 3843 | CTGACC |
| 3844 | GAGACC |
| 3845 | TGGACA |
| 3846 | AAGGCT |
| 3847 | CACTTC |
| 3848 | AATGGC |
| 3849 | CATCAG |
| 3850 | CACTGG |
| 3851 | CTGAGC |
| 3852 | ATCCTC |
| 3853 | CAGGCC |
| 3854 | AACAGG |
| 3855 | CAGAAC |
| 3856 | ATCTGC |
| 3857 | ACTTGG |
| 3858 | CAAGAC |
| 3859 | CTTGGA |
| 3860 | AGCTAC |
| 3861 | AGCTTC |
| 3862 | GCAACA |
| 3863 | ACACCT |
| 3864 | ACCTGA |
| 3865 | GAGCAA |
| 3866 | GCCAAA |
| 3867 | GAAAGC |
| 3868 | CACCAA |
| 3869 | CCCTGA |
| 3870 | AACTGG |
| 3871 | CCATCC |
| 3872 | CTTGCA |
| 3873 | CAGGGC |
| 3874 | GGAACA |
| 3875 | CTTCAC |
| 3876 | ACCACT |
| 3877 | CTGACA |
| 3878 | GCAGCC |
| 3879 | CCATGA |
| 3880 | AAGGTG |
| 3881 | CACCAG |
| 3882 | ACAGAC |
| 3883 | GTGACA |
| 3884 | GCTTCC |
| 3885 | GAACTC |
| 3886 | AGACTC |
| 3887 | AGTCAG |
| 3888 | AAGAGC |
| 3889 | CCCTCA |
| 3890 | CACATC |
| 3891 | GCCACA |
| 3892 | AAGCCT |
| 3893 | AGTAGC |
| 3894 | AGTCTG |
| 3895 | CTGGTC |
| 3896 | GGGCAA |
| 3897 | ATGTGC |
| 3898 | GATCAC |
| 3899 | CCTCAC |
| 3900 | GCATGA |
| 3901 | CCTTGA |
| 3902 | TGCCAA |
| 3903 | CCATGC |
| 3904 | AACCAG |
| 3905 | GCTTCA |
| 3906 | ACAAGG |
| 3907 | GTGCCA |
| 3908 | CTCATC |
| 3909 | CTCACC |
| 3910 | AGGCAC |
| 3911 | AGCTGC |
| 3912 | AGCTCC |
| 3913 | GCTTGA |
| 3914 | ATTCCC |
| 3915 | AACAGC |
| 3916 | CCAAAC |
| 3917 | GAGAAC |
| 3918 | AGCCCT |
| 3919 | CAGTCA |
| 3920 | AGGAGC |
| 3921 | ATCTGG |
| 3922 | CAATGG |
| 3923 | CACAAG |
| 3924 | AGGGAT |
| 3925 | GAATGC |
| 3926 | ACCAGA |
| 3927 | ATCCAC |
| 3928 | GTCCCA |
| 3929 | GCCTGA |
| 3930 | GCCAGA |
| 3931 | GACCTC |
| 3932 | AGGAAC |
| 3933 | CATGGC |
| 3934 | ACCTTG |
| 3935 | GAGTGC |
| 3936 | AACTGC |
| 3937 | ACATGC |
| 3938 | ACCCAA |
| 3939 | GGATCA |
| 3940 | ACCACC |
| 3941 | AAGACC |
| 3942 | CAAACC |
| 3943 | GGGTGA |
| 3944 | GGCTAA |
| 3945 | ACCAGG |
| 3946 | AGGGTG |
| 3947 | CAGTTC |
| 3948 | GAGATC |
| 3949 | GAAGAC |
| 3950 | ACAGCC |
| 3951 | ATCACC |
| 3952 | CAAGGC |
| 3953 | ACCATC |
| 3954 | AATGCC |
| 3955 | ACCTGC |
| 3956 | GAAGGC |
| 3957 | AACCAC |
| 3958 | GCTCCA |
| 3959 | AACCCT |
| 3960 | CATTGC |
| 3961 | CTGTCC |
| 3962 | GGATGA |
| 3963 | GTTGCA |
| 3964 | CATGGG |
| 3965 | GCTCAA |
| 3966 | CAACTC |
| 3967 | GCAGGC |
| 3968 | GGAGCA |
| 3969 | ACCAAG |
| 3970 | CTACTC |
| 3971 | AGCTTG |
| 3972 | ATGAGC |
| 3973 | AGAGCC |
| 3974 | AGGACT |
| 3975 | ACTCAC |
| 3976 | CATCCC |
| 3977 | AAGGAC |
| 3978 | GTTCCA |
| 3979 | GGAGAC |
| 3980 | GCTGCC |
| 3981 | AGTCCC |
| 3982 | GGGACA |
| 3983 | TGACCA |
| 3984 | CCACTC |
| 3985 | GTCACA |
| 3986 | CATGTC |
| 3987 | ATGTCC |
| 3988 | AAGCAC |
| 3989 | CAACAC |
| 3990 | GAAGCC |
| 3991 | TACCCA |
| 3992 | GCACCA |
| 3993 | AGATGC |
| 3994 | ACACTC |
| 3995 | CAGTGC |
| 3996 | CTCCTA |
| 3997 | AGAGTC |
| 3998 | ACCCAC |
| 3999 | AAGCTC |
| 4000 | GTCAGA |
| 4001 | ACTCTC |
| 4002 | GCACAC |
| 4003 | AGACCT |
| 4004 | CAGGTA |
| 4005 | ACCTTC |
| 4006 | CTTGCC |
| 4007 | CTGCAG |
| 4008 | GAACCA |
| 4009 | TAGGCA |
| 4010 | AGTCCA |
| 4011 | CTAGGA |
| 4012 | CAACCA |
| 4013 | AGTTCC |
| 4014 | AGTCAC |
| 4015 | AGGCTC |
| 4016 | GAAGTC |
| 4017 | GAGCCC |
| 4018 | ATCAGG |
| 4019 | AGCATC |
| 4020 | TGGCCA |
| 4021 | CAGGAC |
| 4022 | GGTGCA |
| 4023 | CCAGAC |
| 4024 | AAGGCC |
| 4025 | GTTGGA |
| 4026 | GCAAAC |
| 4027 | CAACTG |
| 4028 | CTGAAC |
| 4029 | GAGTCA |
| 4030 | ATGCAC |
| 4031 | GCACAA |
| 4032 | CAAGGG |
| 4033 | CAGATC |
| 4034 | CTGCTA |
| 4035 | GCCAGC |
| 4036 | AGGTGC |
| 4037 | CCAGTC |
| 4038 | CTACCA |
| 4039 | AGTTGG |
| 4040 | CCTTGC |
| 4041 | CTTGTC |
| 4042 | AGTGAC |
| 4043 | GACTGA |
| 4044 | AGGTTC |
| 4045 | GAACAC |
| 4046 | GACTCA |
| 4047 | ATGCCC |
| 4048 | CAGTAG |
| 4049 | GTGCAA |
| 4050 | TAGGGA |
| 4051 | GGCCCA |
| 4052 | AGGGAC |
| 4053 | GGACAA |
| 4054 | AGGTAG |
| 4055 | ACTGCC |
| 4056 | ACTGTC |
| 4057 | GGGCCA |
| 4058 | AGAACC |
| 4059 | CCTAGA |
| 4060 | AGCAAC |
| 4061 | ACTCCC |
| 4062 | GAGAGC |
| 4063 | GATGCA |
| 4064 | AGACCC |
| 4065 | ACCCAT |
| 4066 | AAGTCC |
| 4067 | AAGCCC |
| 4068 | CCTCTA |
| 4069 | GCCCAC |
| 4070 | AACACC |
| 4071 | AGGCCC |
| 4072 | ATCAGC |
| 4073 | GTCTGA |
| 4074 | ACTTGC |
| 4075 | ACATCC |
| 4076 | CTAGAG |
| 4077 | CCAAGC |
| 4078 | GATTCC |
| 4079 | CTGGAC |
| 4080 | GATCCA |
| 4081 | GTGTCA |
| 4082 | GAACCC |
| 4083 | CAAGTC |
| 4084 | GCATCA |
| 4085 | ATGCTC |
| 4086 | GGCAAC |
| 4087 | GGAGCC |
| 4088 | ACAGTC |
| 4089 | GCTCCC |
| 4090 | GGTGAC |
| 4091 | CATTGG |
| 4092 | GACTCC |
| 4093 | CTCAAC |
| 4094 | GCAACC |
| 4095 | AGTGTC |
| 4096 | CACAAC |
| 4097 | AGCACC |
| 4098 | ACTGGC |
| 4099 | CCAGTA |
| 4100 | ACACCC |
| 4101 | AAGGGT |
| 4102 | ACCCTC |
| 4103 | GAGGGC |
| 4104 | GGTAGA |
| 4105 | ACCAAC |
| 4106 | AAGGGC |
| 4107 | AGGCTA |
| 4108 | ATGGCC |
| 4109 | ACCAGT |
| 4110 | AGGATC |
| 4111 | CCCAAC |
| 4112 | AGGGCC |
| 4113 | CAAGCC |
| 4114 | AAGGTC |
| 4115 | GACCAA |
| 4116 | CCTACA |
| 4117 | ATTGCC |
| 4118 | CAGACC |
| 4119 | ACAAGC |
| 4120 | CAGTCC |
| 4121 | GGACAC |
| 4122 | GCCAAC |
| 4123 | GCTGTA |
| 4124 | CTGGTA |
| 4125 | GGCCAC |
| 4126 | GATGGC |
| 4127 | CAATGC |
| 4128 | CACCTA |
| 4129 | GATTGC |
| 4130 | AGTGCC |
| 4131 | CATGAC |
| 4132 | GACCCA |
| 4133 | GAGGAC |
| 4134 | CAGCTG |
| 4135 | CCCTAA |
| 4136 | CAATCC |
| 4137 | GTGAAC |
| 4138 | CCAACC |
| 4139 | ACTGAC |
| 4140 | CAGGTC |
| 4141 | AGCCTA |
| 4142 | GAATCC |
| 4143 | AGGGTC |
| 4144 | GACAGC |
| 4145 | CTAGCA |
| 4146 | AGTAGG |
| 4147 | GAGGTA |
| 4148 | ATGGAC |
| 4149 | GCCCAA |
| 4150 | ATTGGG |
| 4151 | ATGGGC |
| 4152 | CATAGG |
| 4153 | GTGGTA |
| 4154 | CCTAAG |
| 4155 | GGGCAC |
| 4156 | ACAACC |
| 4157 | CTGTAC |
| 4158 | CCACTA |
| 4159 | GGCACC |
| 4160 | AGCTAG |
| 4161 | GGGAAC |
| 4162 | CTGATC |
| 4163 | GCAGAC |
| 4164 | ATGGTC |
| 4165 | ATTGGC |
| 4166 | GGGTCA |
| 4167 | CAACCC |
| 4168 | AGATCC |
| 4169 | CTTGAC |
| 4170 | GGACTA |
| 4171 | CCTTAG |
| 4172 | GCAGTA |
| 4173 | CCCATA |
| 4174 | GAGCTA |
| 4175 | GATGAC |
| 4176 | GGTTGA |
| 4177 | GCTACA |
| 4178 | GCACCC |
| 4179 | GGCTTA |
| 4180 | GGGATA |
| 4181 | GGTCAA |
| 4182 | AGGGTA |
| 4183 | ATGACC |
| 4184 | ACCTAG |
| 4185 | GTAGGA |
| 4186 | GCTAGA |
| 4187 | CATAGC |
| 4188 | GTCCAA |
| 4189 | AGTTGC |
| 4190 | GGTACA |
| 4191 | GAGCAC |
| 4192 | GCCATA |
| 4193 | CCATAG |
| 4194 | CTTAGC |
| 4195 | GATGCC |
| 4196 | GCTCTA |
| 4197 | AGGACC |
| 4198 | CCCTTA |
| 4199 | ATAGGC |
| 4200 | CACGCC |
| 4201 | GGCATA |
| 4202 | GCATCC |
| 4203 | GTGCTA |
| 4204 | CCAATC |
| 4205 | GACATC |
| 4206 | CAGGCG |
| 4207 | GTAGCA |
| 4208 | GGACCA |
| 4209 | GACTGC |
| 4210 | GGGTAA |
| 4211 | CTAGGC |
| 4212 | GCAAGC |
| 4213 | AGGCCT |
| 4214 | GGAGTA |
| 4215 | GACCAC |
| 4216 | GGAACC |
| 4217 | ACCCTA |
| 4218 | GAGTCC |
| 4219 | GCTGAC |
| 4220 | AGGTCC |
| 4221 | CCTACC |
| 4222 | GGTCAC |
| 4223 | GCCTTA |
| 4224 | GTACCA |
| 4225 | CTTACC |
| 4226 | GACTAC |
| 4227 | CTATGC |
| 4228 | ACCTAC |
| 4229 | CTACAC |
| 4230 | CTAGAC |
| 4231 | ATACCC |
| 4232 | GACACC |
| 4233 | CACTAC |
| 4234 | ATAGGG |
| 4235 | GGGACC |
| 4236 | CTAAGC |
| 4237 | CCCTAG |
| 4238 | ATAGCC |
| 4239 | CATACC |
| 4240 | GCCTAA |
| 4241 | CCTTAC |
| 4242 | GGTCCA |
| 4243 | CTATCC |
| 4244 | CCTAGC |
| 4245 | ACTAGG |
| 4246 | GACAAC |
| 4247 | CAGTAC |
| 4248 | CTAGCC |
| 4249 | CCTAAC |
| 4250 | GGTGTA |
| 4251 | AGGTAC |
| 4252 | CGGGAG |
| 4253 | CACTAG |
| 4254 | GTCCAC |
| 4255 | GATCCC |
| 4256 | AGGCGG |
| 4257 | GGACCC |
| 4258 | CTACCC |
| 4259 | CTCGGC |
| 4260 | CCTATC |
| 4261 | CCATAC |
| 4262 | GGCCTA |
| 4263 | CCCTAC |
| 4264 | CCATGG |
| 4265 | GGGTTA |
| 4266 | ACTACC |
| 4267 | GTCCTA |
| 4268 | GCACTA |
| 4269 | GCTAAC |
| 4270 | GTACAC |
| 4271 | GACCTA |
| 4272 | AGTACC |
| 4273 | CTAACC |
| 4274 | GGTCTA |
| 4275 | GGATAC |
| 4276 | GCTTAC |
| 4277 | GGGCTA |
| 4278 | GTCAAC |
| 4279 | CGCCTC |
| 4280 | CTAGTC |
| 4281 | GATACC |
| 4282 | GCCCTA |
| 4283 | GGTAAC |
| 4284 | GATAGC |
| 4285 | AGACGG |
| 4286 | GGCTAC |
| 4287 | CGTCTC |
| 4288 | ACTAGC |
| 4289 | GCATAC |
| 4290 | GGTTAC |
| 4291 | GAGTAC |
| 4292 | CGAGAC |
| 4293 | GTAGAC |
| 4294 | AGGCCG |
| 4295 | GCCTAC |
| 4296 | GCTACC |
| 4297 | CGCCCA |
| 4298 | GCGTGA |
| 4299 | GAGCTC |
| 4300 | CCTAGG |
| 4301 | ACGCCT |
| 4302 | CCGAGG |
| 4303 | GGCGGA |
| 4304 | CGCCAC |
| 4305 | GGGTAC |
| 4306 | CGTGAG |
| 4307 | ACCACG |
| 4308 | GCATGC |
| 4309 | CCACCG |
| 4310 | CCACGC |
| 4311 | CCGAGA |
| 4312 | CCCGAG |
| 4313 | CGGCTC |
| 4314 | CCGGGA |
| 4315 | TCGAGA |
| 4316 | GTGCAC |
| 4317 | CGAGGC |
| 4318 | CCTCCG |
| 4319 | GGCCGA |
| 4320 | CGGGCA |
| 4321 | ACCCGG |
| 4322 | AGCCGA |
| 4323 | CTCCGC |
| 4324 | ATCTCG |
| 4325 | CCCGTC |
| 4326 | CGTGCC |
| 4327 | ATCACG |
| 4328 | AGCCGG |
| 4329 | CACACG |
| 4330 | CACCGC |
| 4331 | TCCCGA |
| 4332 | AGATCG |
| 4333 | CACCCG |
| 4334 | CGTTTC |
| 4335 | ACCTCG |
| 4336 | ACGTGG |
| 4337 | ACGGTG |
| 4338 | ACGGAG |
| 4339 | AAGCGA |
| 4340 | AAACGG |
| 4341 | AGCGAG |
| 4342 | AGGCGC |
| 4343 | CAAGCG |
| 4344 | CTCGAA |
| 4345 | ACTCGG |
| 4346 | ACCGTG |
| 4347 | AGAGCG |
| 4348 | AGCGAT |
| 4349 | CCGCCA |
| 4350 | CACGAG |
| 4351 | ACACGT |
| 4352 | ATGGCG |
| 4353 | AACGTG |
| 4354 | CCCACG |
| 4355 | CGTGAA |
| 4356 | ACACGG |
| 4357 | ACGAGG |
| 4358 | AACACG |
| 4359 | GCGCCA |
| 4360 | ACGCCA |
| 4361 | TCACGA |
| 4362 | GGATCC |
| 4363 | GCGAGA |
| 4364 | AACCCG |
| 4365 | CAGCCG |
| 4366 | ACGTGA |
| 4367 | ACGCCC |
| 4368 | GAGCGA |
| 4369 | CCACGG |
| 4370 | GACGGA |
| 4371 | CGATTC |
| 4372 | ACGGGA |
| 4373 | CGGCCA |
| 4374 | GGCGCA |
| 4375 | ACTCCG |
| 4376 | CACGCA |
| 4377 | GGGCGA |
| 4378 | AATCGC |
| 4379 | AAAGCG |
| 4380 | CGGGAA |
| 4381 | CGGTGA |
| 4382 | CGTGGC |
| 4383 | CGCTGC |
| 4384 | ACGAAG |
| 4385 | ACGTGC |
| 4386 | CCAGCG |
| 4387 | AGTTCG |
| 4388 | CGCACC |
| 4389 | CGTGGA |
| 4390 | CGACAG |
| 4391 | CGGCAG |
| 4392 | CCGCAG |
| 4393 | CGAGTA |
| 4394 | GTTCGA |
| 4395 | AACGGG |
| 4396 | CGTGCA |
| 4397 | ACGCTG |
| 4398 | CAGACG |
| 4399 | CCAACG |
| 4400 | GCGGGA |
| 4401 | CAGCGG |
| 4402 | CGGAGC |
| 4403 | AGCGTG |
| 4404 | CGCTCC |
| 4405 | CTGCGC |
| 4406 | ATCGTG |
| 4407 | CCGTGC |
| 4408 | CCTGCG |
| 4409 | ACCGCA |
| 4410 | ACGGAA |
| 4411 | CACGGC |
| 4412 | AGCCCG |
| 4413 | CACGGG |
| 4414 | CACGGA |
| 4415 | CAACGA |
| 4416 | CACGTA |
| 4417 | AGCGGG |
| 4418 | CGGAAG |
| 4419 | CGAGAA |
| 4420 | CGCACA |
| 4421 | AGAACG |
| 4422 | CGCAGC |
| 4423 | CAAACG |
| 4424 | ACGCAG |
| 4425 | AAGACG |
| 4426 | GGTACC |
| 4427 | CCGGAG |
| 4428 | CGAGGA |
| 4429 | GATCGC |
| 4430 | ACCCGC |
| 4431 | CACGTG |
| 4432 | GCTAGC |
| 4433 | ACACGC |
| 4434 | AAGGCG |
| 4435 | CGGCTA |
| 4436 | ACTGCG |
| 4437 | ACGTCT |
| 4438 | CAGCGA |
| 4439 | CACGTC |
| 4440 | ACGTTG |
| 4441 | GCGACA |
| 4442 | ATCCCG |
| 4443 | AGGGCG |
| 4444 | CCTCGC |
| 4445 | CGGAGA |
| 4446 | CGCCAG |
| 4447 | CCGTGA |
| 4448 | ACGAGA |
| 4449 | ACAGCG |
| 4450 | AGCACG |
| 4451 | ATCCGC |
| 4452 | TCGCCA |
| 4453 | CTCGCC |
| 4454 | CCTTCG |
| 4455 | CCACGA |
| 4456 | CAGCGC |
| 4457 | CCCTCG |
| 4458 | CCCGGA |
| 4459 | CGAGAG |
| 4460 | GGCGAC |
| 4461 | CCCGCA |
| 4462 | CACGAA |
| 4463 | AGACGA |
| 4464 | CGAAGA |
| 4465 | AGCCGC |
| 4466 | CGGAAA |
| 4467 | ATCGAG |
| 4468 | CGCAGA |
| 4469 | CGGATC |
| 4470 | CCGCAC |
| 4471 | CGAAAC |
| 4472 | ATCGCA |
| 4473 | AAACCG |
| 4474 | GCACGC |
| 4475 | CATTCG |
| 4476 | CGTGTC |
| 4477 | CGTTCA |
| 4478 | AACGGA |
| 4479 | GCACGA |
| 4480 | ACGCAC |
| 4481 | ACGATG |
| 4482 | AGGACG |
| 4483 | AACGCA |
| 4484 | CGCTTC |
| 4485 | CGTCCC |
| 4486 | AAGCGG |
| 4487 | AGGCGA |
| 4488 | ACACGA |
| 4489 | ACGGCA |
| 4490 | CCTCGA |
| 4491 | ACGTCA |
| 4492 | CCGCTC |
| 4493 | CATGCG |
| 4494 | AAGCCG |
| 4495 | AACGAG |
| 4496 | CCGGCA |
| 4497 | AAGCGC |
| 4498 | GAGCGC |
| 4499 | AAACGC |
| 4500 | ACGTTC |
| 4501 | ACCGAG |
| 4502 | CGCTGA |
| 4503 | CCGAGC |
| 4504 | CCGTCC |
| 4505 | CGCTCA |
| 4506 | GCGAAA |
| 4507 | GCGGCA |
| 4508 | CCGAAG |
| 4509 | AACGCT |
| 4510 | ACACCG |
| 4511 | AGCGGC |
| 4512 | CGAAAG |
| 4513 | CTCGGA |
| 4514 | CGTGTA |
| 4515 | AGCGAA |
| 4516 | CCATCG |
| 4517 | CCCGAC |
| 4518 | AGCGCC |
| 4519 | CGTTCC |
| 4520 | ATGACG |
| 4521 | CGAGCC |
| 4522 | CACTCG |
| 4523 | CTGCGA |
| 4524 | CTCGTC |
| 4525 | AAGCGT |
| 4526 | ACAACG |
| 4527 | GCCGCA |
| 4528 | AACTCG |
| 4529 | CCGAAA |
| 4530 | CGTCCA |
| 4531 | CTCCGA |
| 4532 | ACGATC |
| 4533 | ACGTCC |
| 4534 | ACCGGG |
| 4535 | CACCGG |
| 4536 | CGGCAC |
| 4537 | AACGTC |
| 4538 | AGACGC |
| 4539 | CCGGAA |
| 4540 | CGTCAG |
| 4541 | ACTTCG |
| 4542 | ACGGCT |
| 4543 | CACCGA |
| 4544 | AGCGGA |
| 4545 | CGTGAC |
| 4546 | CGCAAA |
| 4547 | AGGTCG |
| 4548 | CGATGA |
| 4549 | ACGTAG |
| 4550 | CGTCAC |
| 4551 | CCCGAA |
| 4552 | GATCGA |
| 4553 | CGAGCA |
| 4554 | CGGTGC |
| 4555 | AGCGCA |
| 4556 | ATGCGG |
| 4557 | CATACG |
| 4558 | CCGTCA |
| 4559 | AGACCG |
| 4560 | TCGTCA |
| 4561 | ACGCAT |
| 4562 | ACGGGC |
| 4563 | GGCGAA |
| 4564 | GAACGA |
| 4565 | AGCTCG |
| 4566 | ACGACA |
| 4567 | CCGCAA |
| 4568 | ACGGCC |
| 4569 | GCGCAC |
| 4570 | ACATCG |
| 4571 | GCCGGA |
| 4572 | CATCGC |
| 4573 | TCCGCA |
| 4574 | CGGGAC |
| 4575 | CATCGA |
| 4576 | ACCGCC |
| 4577 | ATTCCG |
| 4578 | CATCCG |
| 4579 | ACCCGT |
| 4580 | CTTCGA |
| 4581 | TCGGAA |
| 4582 | ACGCAA |
| 4583 | ATTGCG |
| 4584 | ACGAGT |
| 4585 | GAACGC |
| 4586 | ACCCGA |
| 4587 | ATTCGG |
| 4588 | TCGGCA |
| 4589 | AATGCG |
| 4590 | CCGACC |
| 4591 | CCGTTC |
| 4592 | ATGCCG |
| 4593 | ATCGCC |
| 4594 | ACCGAA |
| 4595 | AACCGA |
| 4596 | TCCGAA |
| 4597 | ACCGCT |
| 4598 | CGTAAG |
| 4599 | ACTCGA |
| 4600 | CGTTGA |
| 4601 | AACCGT |
| 4602 | GCGTCC |
| 4603 | CTCGCA |
| 4604 | GCGGAA |
| 4605 | CGGCAA |
| 4606 | CGAAGC |
| 4607 | GCCCGA |
| 4608 | ACGCTC |
| 4609 | AGCGTC |
| 4610 | GTGCGA |
| 4611 | CGGACA |
| 4612 | AACGGT |
| 4613 | CGAACA |
| 4614 | ACGGAT |
| 4615 | GACGCA |
| 4616 | CTTCGC |
| 4617 | AGTGCG |
| 4618 | CCGTAA |
| 4619 | CGGGTC |
| 4620 | CGTAGA |
| 4621 | CGTTGC |
| 4622 | CGCCAA |
| 4623 | AGCGCT |
| 4624 | GACGAA |
| 4625 | GACGCC |
| 4626 | CGAGTC |
| 4627 | ACCGTA |
| 4628 | CGATCC |
| 4629 | GCCGAA |
| 4630 | ATACGG |
| 4631 | CGATCA |
| 4632 | CGCAAG |
| 4633 | GCGAGC |
| 4634 | AAGTCG |
| 4635 | CACGAC |
| 4636 | CCGACA |
| 4637 | AACGCC |
| 4638 | ACGGTA |
| 4639 | TACGCA |
| 4640 | AGTCGG |
| 4641 | ACTACG |
| 4642 | CGGTTC |
| 4643 | ATGCGC |
| 4644 | ACCGCG |
| 4645 | AATCCG |
| 4646 | GACGGC |
| 4647 | ACCGTC |
| 4648 | CAGTCG |
| 4649 | CGCCTA |
| 4650 | CGTCTA |
| 4651 | ACGTAC |
| 4652 | CGCATC |
| 4653 | CGGTCA |
| 4654 | CGTACA |
| 4655 | CAACGG |
| 4656 | TGCGAA |
| 4657 | CGATGC |
| 4658 | ATGTCG |
| 4659 | CGACTC |
| 4660 | CCTACG |
| 4661 | GGACGA |
| 4662 | ACCGGC |
| 4663 | CGACAA |
| 4664 | TACGGA |
| 4665 | CCGGAC |
| 4666 | AACGGC |
| 4667 | CGTCAA |
| 4668 | CGTATC |
| 4669 | AACCGC |
| 4670 | CATCGG |
| 4671 | CGTTAG |
| 4672 | ATGCGA |
| 4673 | GCGCAA |
| 4674 | AATCGG |
| 4675 | ACGAGC |
| 4676 | ATCGTC |
| 4677 | CTCGAC |
| 4678 | AGTCCG |
| 4679 | TCGCAA |
| 4680 | GCGTCA |
| 4681 | ACGACT |
| 4682 | AACCGG |
| 4683 | CGGTCC |
| 4684 | CGGGTA |
| 4685 | ACGGAC |
| 4686 | CGACCC |
| 4687 | TCGACA |
| 4688 | CGACAC |
| 4689 | ACGAAC |
| 4690 | GCTCGA |
| 4691 | CTACGA |
| 4692 | CCGGTC |
| 4693 | CGAATC |
| 4694 | CAACGC |
| 4695 | AGCGAC |
| 4696 | CCGTAG |
| 4697 | ACTCGC |
| 4698 | ATCGGG |
| 4699 | CGGTAA |
| 4700 | AACGAC |
| 4701 | AGTCGA |
| 4702 | ATCCGG |
| 4703 | CCCGTA |
| 4704 | ACGCTA |
| 4705 | CAACCG |
| 4706 | ACGGTC |
| 4707 | GACGTA |
| 4708 | ATAGCG |
| 4709 | CGACCA |
| 4710 | CAATCG |
| 4711 | CGGAAC |
| 4712 | CGGTAG |
| 4713 | CTCGTA |
| 4714 | ATCCGA |
| 4715 | GACCGA |
| 4716 | ACCGGA |
| 4717 | CGCATA |
| 4718 | ATACGC |
| 4719 | AGCGTA |
| 4720 | GACCGC |
| 4721 | CGCTTA |
| 4722 | CGCTAA |
| 4723 | CCGTTA |
| 4724 | ATTCGC |
| 4725 | CGTAGC |
| 4726 | TAGCGA |
| 4727 | CGCAAC |
| 4728 | CGGACC |
| 4729 | ATCGGA |
| 4730 | CGTAAC |
| 4731 | CGATAG |
| 4732 | GCCGAC |
| 4733 | CTCGAG |
| 4734 | ACCGAT |
| 4735 | ATACCG |
| 4736 | CTACGC |
| 4737 | AGTACG |
| 4738 | GTCGAA |
| 4739 | GGCGTA |
| 4740 | GCGGAC |
| 4741 | TACCGA |
| 4742 | CCGCTA |
| 4743 | GGTCGA |
| 4744 | GCGATA |
| 4745 | ACGACC |
| 4746 | AGTCGC |
| 4747 | GCGCGA |
| 4748 | CGTTAC |
| 4749 | ATCGGC |
| 4750 | CGGATA |
| 4751 | GCGTAA |
| 4752 | CGAACC |
| 4753 | GCGTTA |
| 4754 | GACGAC |
| 4755 | CGCTAC |
| 4756 | GCGACC |
| 4757 | CGGTTA |
| 4758 | CCGAAC |
| 4759 | GTCGGA |
| 4760 | GTCGCA |
| 4761 | GCCGTA |
| 4762 | CCGGTA |
| 4763 | CGTACC |
| 4764 | ACCGAC |
| 4765 | CCGATC |
| 4766 | ATCGCG |
| 4767 | CGCTAG |
| 4768 | GTCCGA |
| 4769 | TCCGGA |
| 4770 | CCGATA |
| 4771 | ATCGAC |
| 4772 | GCGGTA |
| 4773 | GCGCTA |
| 4774 | CACGCG |
| 4775 | GTACGA |
| 4776 | TGCGCA |
| 4777 | GTCGTA |
| 4778 | CGACTA |
| 4779 | CCGTAC |
| 4780 | CGATAC |
| 4781 | CGGTAC |
| 4782 | CGCACG |
| 4783 | GCGAAC |
| 4784 | CGCGCA |
| 4785 | AGCGCG |
| 4786 | ACGCGG |
| 4787 | ACGGCG |
| 4788 | GACGTC |
| 4789 | CGAGCG |
| 4790 | CGGACG |
| 4791 | ACGCCG |
| 4792 | ACGCGC |
| 4793 | GCGTAC |
| 4794 | CGCGAG |
| 4795 | CGCGGA |
| 4796 | CGCGTC |
| 4797 | CGGCGA |
| 4798 | CCGCGA |
| 4799 | CGCCGA |
| 4800 | ACCGGT |
| 4801 | CCGACG |
| 4802 | CCGTCG |
| 4803 | CGTCGC |
| 4804 | CGACGC |
| 4805 | ACGACG |
| 4806 | AACGCG |
| 4807 | ACGTCG |
| 4808 | CGCGAC |
| 4809 | CGAACG |
| 4810 | CGACCG |
| 4811 | ACGCGA |
| 4812 | CGCGAA |
| 4813 | CGACGA |
| 4814 | GTCGAC |
| 4815 | CGTCGA |
| 4816 | CGCGTA |
| 4817 | ACGCGT |
| 4818 | TCGCGA |
| 4819 | CGATCG |
| 4820 | CGTACG |
| 4821 | AGGCTG |
| 4822 | CACACA |
| 4823 | CCTCCC |
| 4824 | CAGGAG |
| 4825 | CTGGGA |
| 4826 | CCCAGC |
| 4827 | ACACAC |
| 4828 | CTCCCA |
| 4829 | CCCAGG |
| 4830 | AGGCAG |
| 4831 | ACAGAG |
| 4832 | AGAGAG |
| 4833 | CCAGGC |
| 4834 | GCCTCC |
| 4835 | CCAGCC |
| 4836 | AGGAAG |
| 4837 | AGGAGA |
| 4838 | CCTCAG |
| 4839 | GAGAGA |
| 4840 | AGCCTG |
| 4841 | GGAGAA |
| 4842 | CAGAGA |
| 4843 | ACTGCA |
| 4844 | CTCAGC |
| 4845 | AGACAG |
| 4846 | CACTGC |
| 4847 | AGGAGG |
| 4848 | CCCAAA |
| 4849 | CTGAGA |
| 4850 | CTCCAG |
| 4851 | CAGGCA |
| 4852 | AGCTGG |
| 4853 | AGTGAG |
| 4854 | CACAGA |
| 4855 | TCAGGA |
| 4856 | CAGGAA |
| 4857 | CCACCA |
| 4858 | CCTGCC |
| 4859 | CAGTGA |
| 4860 | CTGGGC |
| 4861 | CCTGGC |
| 4862 | TCCCAA |
| 4863 | CCAGGA |
| 4864 | CAGAAG |
| 4865 | CCAAAG |
| 4866 | CCAGCA |
| 4867 | GAGGCA |
| 4868 | ATCCCA |
| 4869 | AGCCTC |
| 4870 | CAGTGG |
| 4871 | GGCTCA |
| 4872 | GACAGA |
| 4873 | TGGGAA |
| 4874 | GGAAGA |
| 4875 | AGGGAA |
| 4876 | GGGAAA |
| 4877 | CTGGAA |
| 4878 | TCTCCA |
| 4879 | GAGGAA |
| 4880 | CACCTG |
| 4881 | GAAGGA |
| 4882 | ACACAG |
| 4883 | GGCTGA |
| 4884 | GCCTCA |
| 4885 | AGAGGA |
| 4886 | GAGACA |
| 4887 | GCTGGA |
| 4888 | CTTTCC |
| 4889 | CTGCAA |
| 4890 | CTTCTC |
| 4891 | CCTGAG |
| 4892 | CCTGTA |
| 4893 | CAGAGG |
| 4894 | AGATGG |
| 4895 | ATGGTG |
| 4896 | CACCAC |
| 4897 | ACTCCA |
| 4898 | ACCCAG |
| 4899 | TGCCCA |
| 4900 | AGAAGG |
| 4901 | CTTCCA |
| 4902 | AGCCAG |
| 4903 | CCTTCC |
| 4904 | GGAGGA |
| 4905 | AGGGAG |
| 4906 | CTGTGA |
| 4907 | ACTCTG |
| 4908 | CAGCAG |
| 4909 | CCAGAA |
| 4910 | GGCAGA |
| 4911 | CTCAGA |
| 4912 | TCTGCA |
| 4913 | GTCTCA |
| 4914 | CTCTGC |
| 4915 | AAGGAG |
| 4916 | GAGCCA |
| 4917 | AGGTGG |
| 4918 | CATCTC |
| 4919 | AGCAGA |
| 4920 | ACAGGC |
| 4921 | AAGCAG |
| 4922 | CTTCCC |
| 4923 | CCTTTC |
| 4924 | AATCCC |
| 4925 | AGAGCA |
| 4926 | GCAGGA |
| 4927 | GCAGAA |
| 4928 | TCCTCA |
| 4929 | CCCACC |
| 4930 | CTCTTC |
| 4931 | CCTCCA |
| 4932 | CAGCCA |
| 4933 | AAGGGA |
| 4934 | CTCACA |
| 4935 | AAGGCA |
| 4936 | AGAGAC |
| 4937 | CAGGGA |
| 4938 | CAGCAC |
| 4939 | ACAGCA |
| 4940 | ACATGG |
| 4941 | CTGGCC |
| 4942 | TCACCA |
| 4943 | ACAGTG |
| 4944 | AAGAGG |
| 4945 | CACCCA |
| 4946 | CTCTGA |
| 4947 | GGGAGA |
| 4948 | AGACCA |
| 4949 | AGCCAC |
| 4950 | AGCACT |
| 4951 | ACCACA |
| 4952 | CACATG |
| 4953 | CTCCTC |
| 4954 | CAGAGC |
| 4955 | CAGCAA |
| 4956 | AAGCCA |
| 4957 | CCCAGA |
| 4958 | CCACAG |
| 4959 | CTGAAG |
| 4960 | CAGCTA |
| 4961 | GCTCAC |
| 4962 | CTCTCC |
| 4963 | AGGCAT |
| 4964 | ACCTCA |
| 4965 | CCCTCC |
| 4966 | CCTGGA |
| 4967 | ACCATG |
| 4968 | CAGACA |
| 4969 | CCTCTC |
| 4970 | TCCAGA |
| 4971 | AGCTGA |
| 4972 | CAAAGG |
| 4973 | ATGGAG |
| 4974 | AGAGTG |
| 4975 | GTGAGA |
| 4976 | AGGCCA |
| 4977 | CTGCAC |
| 4978 | ACTCCT |
| 4979 | AAACCC |
| 4980 | AAAGGG |
| 4981 | TCAGCA |
| 4982 | CCAGAG |
| 4983 | CCAACA |
| 4984 | AGCCAA |
| 4985 | GAGGGA |
| 4986 | GAAGCA |
| 4987 | CAGATG |
| 4988 | GAGGCC |
| 4989 | ACTGTG |
| 4990 | CATGCC |
| 4991 | CCTGAA |
| 4992 | CCAAGA |
| 4993 | CTCTCA |
| 4994 | TGCACA |
| 4995 | ATGCTG |
| 4996 | CCTCAA |
| 4997 | AACTCC |
| 4998 | CTGCCA |
| 4999 | CACTCC |
| 5000 | AGGTGA |
| 5001 | CACAGG |
| 5002 | AGCAAG |
| 5003 | AAGCTG |
| 5004 | ACAGGA |
| 5005 | CACCTC |
| 5006 | AGCAGG |
| 5007 | AGGTCA |
| 5008 | GCCACC |
| 5009 | AGTGCA |
| 5010 | AGAAGC |
| 5011 | AGGCAA |
| 5012 | ACACTG |
| 5013 | CCATTC |
| 5014 | CACAGC |
| 5015 | CTGCCC |
| 5016 | CTCAAG |
| 5017 | ACTCAG |
| 5018 | CTGCTC |
| 5019 | TCCACA |
| 5020 | ATGGGA |
| 5021 | CAAGTG |
| 5022 | GGAAAC |
| 5023 | AGGACA |
| 5024 | CAGCTC |
| 5025 | CAAGGA |
| 5026 | GAAACC |
| 5027 | ATGGCA |
| 5028 | CCTTCA |
| 5029 | AGCTCA |
| 5030 | CTGTCA |
| 5031 | CATGAG |
| 5032 | CACACC |
| 5033 | CTCCAA |
| 5034 | GACACA |
| 5035 | ACCTGG |
| 5036 | CACTGA |
| 5037 | AAGTGG |
| 5038 | CAAGAG |
| 5039 | ATGTGG |
| 5040 | AGGATG |
| 5041 | ACCTCC |
| 5042 | GATGGA |
| 5043 | AGAGGG |
| 5044 | CCACCC |
| 5045 | CCAGGG |
| 5046 | CATGGA |
| 5047 | ACCTCT |
| 5048 | GTGGGA |
| 5049 | GTGGCA |
| 5050 | CATTCC |
| 5051 | ACTGAG |
| 5052 | AGTGGC |
| 5053 | AGCACA |
| 5054 | CCATCA |
| 5055 | ATGCAG |
| 5056 | AAGTGC |
| 5057 | CAAAGC |
| 5058 | CTTTGC |
| 5059 | GGTGGA |
| 5060 | GTGGAA |
| 5061 | AGAGCT |
| 5062 | CAACAG |
| 5063 | CCCACA |
| 5064 | GGTGAA |
| 5065 | ACTGCT |
| 5066 | AGTCTC |
| 5067 | GGCAAA |
| 5068 | CCTGCA |
| 5069 | AGGTTG |
| 5070 | GCTGCA |
| 5071 | TGAGCA |
| 5072 | AGTGGA |
| 5073 | AGACTG |
| 5074 | AGCCCA |
| 5075 | AACCTC |
| 5076 | ATGCCA |
| 5077 | CAAGCA |
| 5078 | AACCCA |
| 5079 | GCAGCA |
| 5080 | ACAGGG |
| 5081 | CTCCAC |
| 5082 | GAGTGA |
| 5083 | AGCAGC |
| 5084 | CCCAAG |
| 5085 | GCTGAA |
| 5086 | CTTGGC |
| 5087 | AATGGG |
| 5088 | CCTGAC |
| 5089 | CCACAA |
| 5090 | GGCCAA |
| 5091 | ACACCA |
| 5092 | CTGGCA |
| 5093 | GTGTGA |
| 5094 | TGGCAA |
| 5095 | AGCCAT |
| 5096 | CCAAGG |
| 5097 | ACCAGC |
| 5098 | ATCCTG |
| 5099 | CATCAC |
| 5100 | CCCTGC |
| 5101 | ATGAGG |
| 5102 | CAGCCC |
| 5103 | CATGCA |
| 5104 | GGCACA |
| 5105 | AGAGGC |
| 5106 | CCCTTC |
| 5107 | AGTGTG |
| 5108 | AACCTG |
| 5109 | AGACAC |
| 5110 | AAAGGC |
| 5111 | CCACAC |
| 5112 | GCAAGA |
| 5113 | CCCATC |
| 5114 | CTGTGC |
| 5115 | ACCCTG |
| 5116 | AAAGCC |
| 5117 | CTCTAC |
| 5118 | ACAGGT |
| 5119 | ACTGGA |
| 5120 | ACAGCT |
| 5121 | CATCCA |
| 5122 | ACTGGG |
| 5123 | CACTCA |
| 5124 | ATCCAG |
| 5125 | CTGTTC |
| 5126 | ATCTCC |
| 5127 | CCTGTC |
| 5128 | CTACAG |
| 5129 | AGTGGG |
| 5130 | GGTTCA |
| 5131 | ACTTCC |
| 5132 | AGCATG |
| 5133 | AGGGCA |
| 5134 | TAGCCA |
| 5135 | CTGACC |
| 5136 | GAGACC |
| 5137 | TGGACA |
| 5138 | AAGGCT |
| 5139 | CACTTC |
| 5140 | AATGGC |
| 5141 | CATCAG |
| 5142 | CACTGG |
| 5143 | CTGAGC |
| 5144 | ATCCTC |
| 5145 | CAGGCC |
| 5146 | AACAGG |
| 5147 | CAGAAC |
| 5148 | ATCTGC |
| 5149 | ACTTGG |
| 5150 | CAAGAC |
| 5151 | CTTGGA |
| 5152 | AGCTAC |
| 5153 | AGCTTC |
| 5154 | GCAACA |
| 5155 | ACACCT |
| 5156 | ACCTGA |
| 5157 | GAGCAA |
| 5158 | GCCAAA |
| 5159 | GAAAGC |
| 5160 | CACCAA |
| 5161 | CCCTGA |
| 5162 | AACTGG |
| 5163 | CCATCC |
| 5164 | CTTGCA |
| 5165 | CAGGGC |
| 5166 | GGAACA |
| 5167 | CTTCAC |
| 5168 | ACCACT |
| 5169 | CTGACA |
| 5170 | GCAGCC |
| 5171 | CCATGA |
| 5172 | AAGGTG |
| 5173 | CACCAG |
| 5174 | ACAGAC |
| 5175 | GTGACA |
| 5176 | GCTTCC |
| 5177 | GAACTC |
| 5178 | AGACTC |
| 5179 | AGTCAG |
| 5180 | AAGAGC |
| 5181 | CCCTCA |
| 5182 | CACATC |
| 5183 | GCCACA |
| 5184 | AAGCCT |
| 5185 | AGTAGC |
| 5186 | AGTCTG |
| 5187 | CTGGTC |
| 5188 | GGGCAA |
| 5189 | ATGTGC |
| 5190 | GATCAC |
| 5191 | CCTCAC |
| 5192 | GCATGA |
| 5193 | CCTTGA |
| 5194 | TGCCAA |
| 5195 | CCATGC |
| 5196 | AACCAG |
| 5197 | GCTTCA |
| 5198 | ACAAGG |
| 5199 | GTGCCA |
| 5200 | CTCATC |
| 5201 | CTCACC |
| 5202 | AGGCAC |
| 5203 | AGCTGC |
| 5204 | AGCTCC |
| 5205 | GCTTGA |
| 5206 | ATTCCC |
| 5207 | AACAGC |
| 5208 | CCAAAC |
| 5209 | GAGAAC |
| 5210 | AGCCCT |
| 5211 | CAGTCA |
| 5212 | AGGAGC |
| 5213 | ATCTGG |
| 5214 | CAATGG |
| 5215 | CACAAG |
| 5216 | AGGGAT |
| 5217 | GAATGC |
| 5218 | ACCAGA |
| 5219 | ATCCAC |
| 5220 | GTCCCA |
| 5221 | GCCTGA |
| 5222 | GCCAGA |
| 5223 | GACCTC |
| 5224 | AGGAAC |
| 5225 | CATGGC |
| 5226 | ACCTTG |
| 5227 | GAGTGC |
| 5228 | AACTGC |
| 5229 | ACATGC |
| 5230 | ACCCAA |
| 5231 | GGATCA |
| 5232 | ACCACC |
| 5233 | AAGACC |
| 5234 | CAAACC |
| 5235 | GGGTGA |
| 5236 | GGCTAA |
| 5237 | ACCAGG |
| 5238 | AGGGTG |
| 5239 | CAGTTC |
| 5240 | GAGATC |
| 5241 | GAAGAC |
| 5242 | ACAGCC |
| 5243 | ATCACC |
| 5244 | CAAGGC |
| 5245 | ACCATC |
| 5246 | AATGCC |
| 5247 | ACCTGC |
| 5248 | GAAGGC |
| 5249 | AACCAC |
| 5250 | GCTCCA |
| 5251 | AACCCT |
| 5252 | CATTGC |
| 5253 | CTGTCC |
| 5254 | GGATGA |
| 5255 | GTTGCA |
| 5256 | CATGGG |
| 5257 | GCTCAA |
| 5258 | CAACTC |
| 5259 | GCAGGC |
| 5260 | GGAGCA |
| 5261 | ACCAAG |
| 5262 | CTACTC |
| 5263 | AGCTTG |
| 5264 | ATGAGC |
| 5265 | AGAGCC |
| 5266 | AGGACT |
| 5267 | ACTCAC |
| 5268 | CATCCC |
| 5269 | AAGGAC |
| 5270 | GTTCCA |
| 5271 | GGAGAC |
| 5272 | GCTGCC |
| 5273 | AGTCCC |
| 5274 | GGGACA |
| 5275 | TGACCA |
| 5276 | CCACTC |
| 5277 | GTCACA |
| 5278 | CATGTC |
| 5279 | ATGTCC |
| 5280 | AAGCAC |
| 5281 | CAACAC |
| 5282 | GAAGCC |
| 5283 | TACCCA |
| 5284 | GCACCA |
| 5285 | AGATGC |
| 5286 | ACACTC |
| 5287 | CAGTGC |
| 5288 | CTCCTA |
| 5289 | AGAGTC |
| 5290 | ACCCAC |
| 5291 | AAGCTC |
| 5292 | GTCAGA |
| 5293 | ACTCTC |
| 5294 | GCACAC |
| 5295 | AGACCT |
| 5296 | CAGGTA |
| 5297 | ACCTTC |
| 5298 | CTTGCC |
| 5299 | CTGCAG |
| 5300 | GAACCA |
| 5301 | TAGGCA |
| 5302 | AGTCCA |
| 5303 | CTAGGA |
| 5304 | CAACCA |
| 5305 | AGTTCC |
| 5306 | AGTCAC |
| 5307 | AGGCTC |
| 5308 | GAAGTC |
| 5309 | GAGCCC |
| 5310 | ATCAGG |
| 5311 | AGCATC |
| 5312 | TGGCCA |
| 5313 | CAGGAC |
| 5314 | GGTGCA |
| 5315 | CCAGAC |
| 5316 | AAGGCC |
| 5317 | GTTGGA |
| 5318 | GCAAAC |
| 5319 | CAACTG |
| 5320 | CTGAAC |
| 5321 | GAGTCA |
| 5322 | ATGCAC |
| 5323 | GCACAA |
| 5324 | CAAGGG |
| 5325 | CAGATC |
| 5326 | CTGCTA |
| 5327 | GCCAGC |
| 5328 | AGGTGC |
| 5329 | CCAGTC |
| 5330 | CTACCA |
| 5331 | AGTTGG |
| 5332 | CCTTGC |
| 5333 | CTTGTC |
| 5334 | AGTGAC |
| 5335 | GACTGA |
| 5336 | AGGTTC |
| 5337 | GAACAC |
| 5338 | GACTCA |
| 5339 | ATGCCC |
| 5340 | CAGTAG |
| 5341 | GTGCAA |
| 5342 | TAGGGA |
| 5343 | GGCCCA |
| 5344 | AGGGAC |
| 5345 | GGACAA |
| 5346 | AGGTAG |
| 5347 | ACTGCC |
| 5348 | ACTGTC |
| 5349 | GGGCCA |
| 5350 | AGAACC |
| 5351 | CCTAGA |
| 5352 | AGCAAC |
| 5353 | ACTCCC |
| 5354 | GAGAGC |
| 5355 | GATGCA |
| 5356 | AGACCC |
| 5357 | ACCCAT |
| 5358 | AAGTCC |
| 5359 | AAGCCC |
| 5360 | CCTCTA |
| 5361 | GCCCAC |
| 5362 | AACACC |
| 5363 | AGGCCC |
| 5364 | ATCAGC |
| 5365 | GTCTGA |
| 5366 | ACTTGC |
| 5367 | ACATCC |
| 5368 | CTAGAG |
| 5369 | CCAAGC |
| 5370 | GATTCC |
| 5371 | CTGGAC |
| 5372 | GATCCA |
| 5373 | GTGTCA |
| 5374 | GAACCC |
| 5375 | CAAGTC |
| 5376 | GCATCA |
| 5377 | ATGCTC |
| 5378 | GGCAAC |
| 5379 | GGAGCC |
| 5380 | ACAGTC |
| 5381 | GCTCCC |
| 5382 | GGTGAC |
| 5383 | CATTGG |
| 5384 | GACTCC |
| 5385 | CTCAAC |
| 5386 | GCAACC |
| 5387 | AGTGTC |
| 5388 | CACAAC |
| 5389 | AGCACC |
| 5390 | ACTGGC |
| 5391 | CCAGTA |
| 5392 | ACACCC |
| 5393 | AAGGGT |
| 5394 | ACCCTC |
| 5395 | GAGGGC |
| 5396 | GGTAGA |
| 5397 | ACCAAC |
| 5398 | AAGGGC |
| 5399 | AGGCTA |
| 5400 | ATGGCC |
| 5401 | ACCAGT |
| 5402 | AGGATC |
| 5403 | CCCAAC |
| 5404 | AGGGCC |
| 5405 | CAAGCC |
| 5406 | AAGGTC |
| 5407 | GACCAA |
| 5408 | CCTACA |
| 5409 | ATTGCC |
| 5410 | CAGACC |
| 5411 | ACAAGC |
| 5412 | CAGTCC |
| 5413 | GGACAC |
| 5414 | GCCAAC |
| 5415 | GCTGTA |
| 5416 | CTGGTA |
| 5417 | GGCCAC |
| 5418 | GATGGC |
| 5419 | CAATGC |
| 5420 | CACCTA |
| 5421 | GATTGC |
| 5422 | AGTGCC |
| 5423 | CATGAC |
| 5424 | GACCCA |
| 5425 | GAGGAC |
| 5426 | CAGCTG |
| 5427 | CCCTAA |
| 5428 | CAATCC |
| 5429 | GTGAAC |
| 5430 | CCAACC |
| 5431 | ACTGAC |
| 5432 | CAGGTC |
| 5433 | AGCCTA |
| 5434 | GAATCC |
| 5435 | AGGGTC |
| 5436 | GACAGC |
| 5437 | CTAGCA |
| 5438 | AGTAGG |
| 5439 | GAGGTA |
| 5440 | ATGGAC |
| 5441 | GCCCAA |
| 5442 | ATTGGG |
| 5443 | ATGGGC |
| 5444 | CATAGG |
| 5445 | GTGGTA |
| 5446 | CCTAAG |
| 5447 | GGGCAC |
| 5448 | ACAACC |
| 5449 | CTGTAC |
| 5450 | CCACTA |
| 5451 | GGCACC |
| 5452 | AGCTAG |
| 5453 | GGGAAC |
| 5454 | CTGATC |
| 5455 | GCAGAC |
| 5456 | ATGGTC |
| 5457 | ATTGGC |
| 5458 | GGGTCA |
| 5459 | CAACCC |
| 5460 | AGATCC |
| 5461 | CTTGAC |
| 5462 | GGACTA |
| 5463 | CCTTAG |
| 5464 | GCAGTA |
| 5465 | CCCATA |
| 5466 | GAGCTA |
| 5467 | GATGAC |
| 5468 | GGTTGA |
| 5469 | GCTACA |
| 5470 | GCACCC |
| 5471 | GGCTTA |
| 5472 | GGGATA |
| 5473 | GGTCAA |
| 5474 | AGGGTA |
| 5475 | ATGACC |
| 5476 | ACCTAG |
| 5477 | GTAGGA |
| 5478 | GCTAGA |
| 5479 | CATAGC |
| 5480 | GTCCAA |
| 5481 | AGTTGC |
| 5482 | GGTACA |
| 5483 | GAGCAC |
| 5484 | GCCATA |
| 5485 | CCATAG |
| 5486 | CTTAGC |
| 5487 | GATGCC |
| 5488 | GCTCTA |
| 5489 | AGGACC |
| 5490 | CCCTTA |
| 5491 | ATAGGC |
| 5492 | CACGCC |
| 5493 | GGCATA |
| 5494 | GCATCC |
| 5495 | GTGCTA |
| 5496 | CCAATC |
| 5497 | GACATC |
| 5498 | CAGGCG |
| 5499 | GTAGCA |
| 5500 | GGACCA |
| 5501 | GACTGC |
| 5502 | GGGTAA |
| 5503 | CTAGGC |
| 5504 | GCAAGC |
| 5505 | AGGCCT |
| 5506 | GGAGTA |
| 5507 | GACCAC |
| 5508 | GGAACC |
| 5509 | ACCCTA |
| 5510 | GAGTCC |
| 5511 | GCTGAC |
| 5512 | AGGTCC |
| 5513 | CCTACC |
| 5514 | GGTCAC |
| 5515 | GCCTTA |
| 5516 | GTACCA |
| 5517 | CTTACC |
| 5518 | GACTAC |
| 5519 | CTATGC |
| 5520 | ACCTAC |
| 5521 | CTACAC |
| 5522 | CTAGAC |
| 5523 | ATACCC |
| 5524 | GACACC |
| 5525 | CACTAC |
| 5526 | ATAGGG |
| 5527 | GGGACC |
| 5528 | CTAAGC |
| 5529 | CCCTAG |
| 5530 | ATAGCC |
| 5531 | CATACC |
| 5532 | GCCTAA |
| 5533 | CCTTAC |
| 5534 | GGTCCA |
| 5535 | CTATCC |
| 5536 | CCTAGC |
| 5537 | ACTAGG |
| 5538 | GACAAC |
| 5539 | CAGTAC |
| 5540 | CTAGCC |
| 5541 | CCTAAC |
| 5542 | GGTGTA |
| 5543 | AGGTAC |
| 5544 | CGGGAG |
| 5545 | CACTAG |
| 5546 | GTCCAC |
| 5547 | GATCCC |
| 5548 | AGGCGG |
| 5549 | GGACCC |
| 5550 | CTACCC |
| 5551 | CTCGGC |
| 5552 | CCTATC |
| 5553 | CCATAC |
| 5554 | GGCCTA |
| 5555 | CCCTAC |
| 5556 | CCATGG |
| 5557 | GGGTTA |
| 5558 | ACTACC |
| 5559 | GTCCTA |
| 5560 | GCACTA |
| 5561 | GCTAAC |
| 5562 | GTACAC |
| 5563 | GACCTA |
| 5564 | AGTACC |
| 5565 | CTAACC |
| 5566 | GGTCTA |
| 5567 | GGATAC |
| 5568 | GCTTAC |
| 5569 | GGGCTA |
| 5570 | GTCAAC |
| 5571 | CGCCTC |
| 5572 | CTAGTC |
| 5573 | GATACC |
| 5574 | GCCCTA |
| 5575 | GGTAAC |
| 5576 | GATAGC |
| 5577 | AGACGG |
| 5578 | GGCTAC |
| 5579 | CGTCTC |
| 5580 | ACTAGC |
| 5581 | GCATAC |
| 5582 | GGTTAC |
| 5583 | GAGTAC |
| 5584 | CGAGAC |
| 5585 | GTAGAC |
| 5586 | AGGCCG |
| 5587 | GCCTAC |
| 5588 | GCTACC |
| 5589 | CGCCCA |
| 5590 | GCGTGA |
| 5591 | GAGCTC |
| 5592 | CCTAGG |
| 5593 | ACGCCT |
| 5594 | CCGAGG |
| 5595 | GGCGGA |
| 5596 | CGCCAC |
| 5597 | GGGTAC |
| 5598 | CGTGAG |
| 5599 | ACCACG |
| 5600 | GCATGC |
| 5601 | CCACCG |
| 5602 | CCACGC |
| 5603 | CCGAGA |
| 5604 | CCCGAG |
| 5605 | CGGCTC |
| 5606 | CCGGGA |
| 5607 | TCGAGA |
| 5608 | GTGCAC |
| 5609 | CGAGGC |
| 5610 | CCTCCG |
| 5611 | GGCCGA |
| 5612 | CGGGCA |
| 5613 | ACCCGG |
| 5614 | AGCCGA |
| 5615 | CTCCGC |
| 5616 | ATCTCG |
| 5617 | CCCGTC |
| 5618 | CGTGCC |
| 5619 | ATCACG |
| 5620 | AGCCGG |
| 5621 | CACACG |
| 5622 | CACCGC |
| 5623 | TCCCGA |
| 5624 | AGATCG |
| 5625 | CACCCG |
| 5626 | CGTTTC |
| 5627 | ACCTCG |
| 5628 | ACGTGG |
| 5629 | ACGGTG |
| 5630 | ACGGAG |
| 5631 | AAGCGA |
| 5632 | AAACGG |
| 5633 | AGCGAG |
| 5634 | AGGCGC |
| 5635 | CAAGCG |
| 5636 | CTCGAA |
| 5637 | ACTCGG |
| 5638 | ACCGTG |
| 5639 | AGAGCG |
| 5640 | AGCGAT |
| 5641 | CCGCCA |
| 5642 | CACGAG |
| 5643 | ACACGT |
| 5644 | ATGGCG |
| 5645 | AACGTG |
| 5646 | CCCACG |
| 5647 | CGTGAA |
| 5648 | ACACGG |
| 5649 | ACGAGG |
| 5650 | AACACG |
| 5651 | GCGCCA |
| 5652 | ACGCCA |
| 5653 | TCACGA |
| 5654 | GGATCC |
| 5655 | GCGAGA |
| 5656 | AACCCG |
| 5657 | CAGCCG |
| 5658 | ACGTGA |
| 5659 | ACGCCC |
| 5660 | GAGCGA |
| 5661 | CCACGG |
| 5662 | GACGGA |
| 5663 | CGATTC |
| 5664 | ACGGGA |
| 5665 | CGGCCA |
| 5666 | GGCGCA |
| 5667 | ACTCCG |
| 5668 | CACGCA |
| 5669 | GGGCGA |
| 5670 | AATCGC |
| 5671 | AAAGCG |
| 5672 | CGGGAA |
| 5673 | CGGTGA |
| 5674 | CGTGGC |
| 5675 | CGCTGC |
| 5676 | ACGAAG |
| 5677 | ACGTGC |
| 5678 | CCAGCG |
| 5679 | AGTTCG |
| 5680 | CGCACC |
| 5681 | CGTGGA |
| 5682 | CGACAG |
| 5683 | CGGCAG |
| 5684 | CCGCAG |
| 5685 | CGAGTA |
| 5686 | GTTCGA |
| 5687 | AACGGG |
| 5688 | CGTGCA |
| 5689 | ACGCTG |
| 5690 | CAGACG |
| 5691 | CCAACG |
| 5692 | GCGGGA |
| 5693 | CAGCGG |
| 5694 | CGGAGC |
| 5695 | AGCGTG |
| 5696 | CGCTCC |
| 5697 | CTGCGC |
| 5698 | ATCGTG |
| 5699 | CCGTGC |
| 5700 | CCTGCG |
| 5701 | ACCGCA |
| 5702 | ACGGAA |
| 5703 | CACGGC |
| 5704 | AGCCCG |
| 5705 | CACGGG |
| 5706 | CACGGA |
| 5707 | CAACGA |
| 5708 | CACGTA |
| 5709 | AGCGGG |
| 5710 | CGGAAG |
| 5711 | CGAGAA |
| 5712 | CGCACA |
| 5713 | AGAACG |
| 5714 | CGCAGC |
| 5715 | CAAACG |
| 5716 | ACGCAG |
| 5717 | AAGACG |
| 5718 | GGTACC |
| 5719 | CCGGAG |
| 5720 | CGAGGA |
| 5721 | GATCGC |
| 5722 | ACCCGC |
| 5723 | CACGTG |
| 5724 | GCTAGC |
| 5725 | ACACGC |
| 5726 | AAGGCG |
| 5727 | CGGCTA |
| 5728 | ACTGCG |
| 5729 | ACGTCT |
| 5730 | CAGCGA |
| 5731 | CACGTC |
| 5732 | ACGTTG |
| 5733 | GCGACA |
| 5734 | ATCCCG |
| 5735 | AGGGCG |
| 5736 | CCTCGC |
| 5737 | CGGAGA |
| 5738 | CGCCAG |
| 5739 | CCGTGA |
| 5740 | ACGAGA |
| 5741 | ACAGCG |
| 5742 | AGCACG |
| 5743 | ATCCGC |
| 5744 | TCGCCA |
| 5745 | CTCGCC |
| 5746 | CCTTCG |
| 5747 | CCACGA |
| 5748 | CAGCGC |
| 5749 | CCCTCG |
| 5750 | CCCGGA |
| 5751 | CGAGAG |
| 5752 | GGCGAC |
| 5753 | CCCGCA |
| 5754 | CACGAA |
| 5755 | AGACGA |
| 5756 | CGAAGA |
| 5757 | AGCCGC |
| 5758 | CGGAAA |
| 5759 | ATCGAG |
| 5760 | CGCAGA |
| 5761 | CGGATC |
| 5762 | CCGCAC |
| 5763 | CGAAAC |
| 5764 | ATCGCA |
| 5765 | AAACCG |
| 5766 | GCACGC |
| 5767 | CATTCG |
| 5768 | CGTGTC |
| 5769 | CGTTCA |
| 5770 | AACGGA |
| 5771 | GCACGA |
| 5772 | ACGCAC |
| 5773 | ACGATG |
| 5774 | AGGACG |
| 5775 | AACGCA |
| 5776 | CGCTTC |
| 5777 | CGTCCC |
| 5778 | AAGCGG |
| 5779 | AGGCGA |
| 5780 | ACACGA |
| 5781 | ACGGCA |
| 5782 | CCTCGA |
| 5783 | ACGTCA |
| 5784 | CCGCTC |
| 5785 | CATGCG |
| 5786 | AAGCCG |
| 5787 | AACGAG |
| 5788 | CCGGCA |
| 5789 | AAGCGC |
| 5790 | GAGCGC |
| 5791 | AAACGC |
| 5792 | ACGTTC |
| 5793 | ACCGAG |
| 5794 | CGCTGA |
| 5795 | CCGAGC |
| 5796 | CCGTCC |
| 5797 | CGCTCA |
| 5798 | GCGAAA |
| 5799 | GCGGCA |
| 5800 | CCGAAG |
| 5801 | AACGCT |
| 5802 | ACACCG |
| 5803 | AGCGGC |
| 5804 | CGAAAG |
| 5805 | CTCGGA |
| 5806 | CGTGTA |
| 5807 | AGCGAA |
| 5808 | CCATCG |
| 5809 | CCCGAC |
| 5810 | AGCGCC |
| 5811 | CGTTCC |
| 5812 | ATGACG |
| 5813 | CGAGCC |
| 5814 | CACTCG |
| 5815 | CTGCGA |
| 5816 | CTCGTC |
| 5817 | AAGCGT |
| 5818 | ACAACG |
| 5819 | GCCGCA |
| 5820 | AACTCG |
| 5821 | CCGAAA |
| 5822 | CGTCCA |
| 5823 | CTCCGA |
| 5824 | ACGATC |
| 5825 | ACGTCC |
| 5826 | ACCGGG |
| 5827 | CACCGG |
| 5828 | CGGCAC |
| 5829 | AACGTC |
| 5830 | AGACGC |
| 5831 | CCGGAA |
| 5832 | CGTCAG |
| 5833 | ACTTCG |
| 5834 | ACGGCT |
| 5835 | CACCGA |
| 5836 | AGCGGA |
| 5837 | CGTGAC |
| 5838 | CGCAAA |
| 5839 | AGGTCG |
| 5840 | CGATGA |
| 5841 | ACGTAG |
| 5842 | CGTCAC |
| 5843 | CCCGAA |
| 5844 | GATCGA |
| 5845 | CGAGCA |
| 5846 | CGGTGC |
| 5847 | AGCGCA |
| 5848 | ATGCGG |
| 5849 | CATACG |
| 5850 | CCGTCA |
| 5851 | AGACCG |
| 5852 | TCGTCA |
| 5853 | ACGCAT |
| 5854 | ACGGGC |
| 5855 | GGCGAA |
| 5856 | GAACGA |
| 5857 | AGCTCG |
| 5858 | ACGACA |
| 5859 | CCGCAA |
| 5860 | ACGGCC |
| 5861 | GCGCAC |
| 5862 | ACATCG |
| 5863 | GCCGGA |
| 5864 | CATCGC |
| 5865 | TCCGCA |
| 5866 | CGGGAC |
| 5867 | CATCGA |
| 5868 | ACCGCC |
| 5869 | ATTCCG |
| 5870 | CATCCG |
| 5871 | ACCCGT |
| 5872 | CTTCGA |
| 5873 | TCGGAA |
| 5874 | ACGCAA |
| 5875 | ATTGCG |
| 5876 | ACGAGT |
| 5877 | GAACGC |
| 5878 | ACCCGA |
| 5879 | ATTCGG |
| 5880 | TCGGCA |
| 5881 | AATGCG |
| 5882 | CCGACC |
| 5883 | CCGTTC |
| 5884 | ATGCCG |
| 5885 | ATCGCC |
| 5886 | ACCGAA |
| 5887 | AACCGA |
| 5888 | TCCGAA |
| 5889 | ACCGCT |
| 5890 | CGTAAG |
| 5891 | ACTCGA |
| 5892 | CGTTGA |
| 5893 | AACCGT |
| 5894 | GCGTCC |
| 5895 | CTCGCA |
| 5896 | GCGGAA |
| 5897 | CGGCAA |
| 5898 | CGAAGC |
| 5899 | GCCCGA |
| 5900 | ACGCTC |
| 5901 | AGCGTC |
| 5902 | GTGCGA |
| 5903 | CGGACA |
| 5904 | AACGGT |
| 5905 | CGAACA |
| 5906 | ACGGAT |
| 5907 | GACGCA |
| 5908 | CTTCGC |
| 5909 | AGTGCG |
| 5910 | CCGTAA |
| 5911 | CGGGTC |
| 5912 | CGTAGA |
| 5913 | CGTTGC |
| 5914 | CGCCAA |
| 5915 | AGCGCT |
| 5916 | GACGAA |
| 5917 | GACGCC |
| 5918 | CGAGTC |
| 5919 | ACCGTA |
| 5920 | CGATCC |
| 5921 | GCCGAA |
| 5922 | ATACGG |
| 5923 | CGATCA |
| 5924 | CGCAAG |
| 5925 | GCGAGC |
| 5926 | AAGTCG |
| 5927 | CACGAC |
| 5928 | CCGACA |
| 5929 | AACGCC |
| 5930 | ACGGTA |
| 5931 | TACGCA |
| 5932 | AGTCGG |
| 5933 | ACTACG |
| 5934 | CGGTTC |
| 5935 | ATGCGC |
| 5936 | ACCGCG |
| 5937 | AATCCG |
| 5938 | GACGGC |
| 5939 | ACCGTC |
| 5940 | CAGTCG |
| 5941 | CGCCTA |
| 5942 | CGTCTA |
| 5943 | ACGTAC |
| 5944 | CGCATC |
| 5945 | CGGTCA |
| 5946 | CGTACA |
| 5947 | CAACGG |
| 5948 | TGCGAA |
| 5949 | CGATGC |
| 5950 | ATGTCG |
| 5951 | CGACTC |
| 5952 | CCTACG |
| 5953 | GGACGA |
| 5954 | ACCGGC |
| 5955 | CGACAA |
| 5956 | TACGGA |
| 5957 | CCGGAC |
| 5958 | AACGGC |
| 5959 | CGTCAA |
| 5960 | CGTATC |
| 5961 | AACCGC |
| 5962 | CATCGG |
| 5963 | CGTTAG |
| 5964 | ATGCGA |
| 5965 | GCGCAA |
| 5966 | AATCGG |
| 5967 | ACGAGC |
| 5968 | ATCGTC |
| 5969 | CTCGAC |
| 5970 | AGTCCG |
| 5971 | TCGCAA |
| 5972 | GCGTCA |
| 5973 | ACGACT |
| 5974 | AACCGG |
| 5975 | CGGTCC |
| 5976 | CGGGTA |
| 5977 | ACGGAC |
| 5978 | CGACCC |
| 5979 | TCGACA |
| 5980 | CGACAC |
| 5981 | ACGAAC |
| 5982 | GCTCGA |
| 5983 | CTACGA |
| 5984 | CCGGTC |
| 5985 | CGAATC |
| 5986 | CAACGC |
| 5987 | AGCGAC |
| 5988 | CCGTAG |
| 5989 | ACTCGC |
| 5990 | ATCGGG |
| 5991 | CGGTAA |
| 5992 | AACGAC |
| 5993 | AGTCGA |
| 5994 | ATCCGG |
| 5995 | CCCGTA |
| 5996 | ACGCTA |
| 5997 | CAACCG |
| 5998 | ACGGTC |
| 5999 | GACGTA |
| 6000 | ATAGCG |
| 6001 | CGACCA |
| 6002 | CAATCG |
| 6003 | CGGAAC |
| 6004 | CGGTAG |
| 6005 | CTCGTA |
| 6006 | ATCCGA |
| 6007 | GACCGA |
| 6008 | ACCGGA |
| 6009 | CGCATA |
| 6010 | ATACGC |
| 6011 | AGCGTA |
| 6012 | GACCGC |
| 6013 | CGCTTA |
| 6014 | CGCTAA |
| 6015 | CCGTTA |
| 6016 | ATTCGC |
| 6017 | CGTAGC |
| 6018 | TAGCGA |
| 6019 | CGCAAC |
| 6020 | CGGACC |
| 6021 | ATCGGA |
| 6022 | CGTAAC |
| 6023 | CGATAG |
| 6024 | GCCGAC |
| 6025 | CTCGAG |
| 6026 | ACCGAT |
| 6027 | ATACCG |
| 6028 | CTACGC |
| 6029 | AGTACG |
| 6030 | GTCGAA |
| 6031 | GGCGTA |
| 6032 | GCGGAC |
| 6033 | TACCGA |
| 6034 | CCGCTA |
| 6035 | GGTCGA |
| 6036 | GCGATA |
| 6037 | ACGACC |
| 6038 | AGTCGC |
| 6039 | GCGCGA |
| 6040 | CGTTAC |
| 6041 | ATCGGC |
| 6042 | CGGATA |
| 6043 | GCGTAA |
| 6044 | CGAACC |
| 6045 | GCGTTA |
| 6046 | GACGAC |
| 6047 | CGCTAC |
| 6048 | GCGACC |
| 6049 | CGGTTA |
| 6050 | CCGAAC |
| 6051 | GTCGGA |
| 6052 | GTCGCA |
| 6053 | GCCGTA |
| 6054 | CCGGTA |
| 6055 | CGTACC |
| 6056 | ACCGAC |
| 6057 | CCGATC |
| 6058 | ATCGCG |
| 6059 | CGCTAG |
| 6060 | GTCCGA |
| 6061 | TCCGGA |
| 6062 | CCGATA |
| 6063 | ATCGAC |
| 6064 | GCGGTA |
| 6065 | GCGCTA |
| 6066 | CACGCG |
| 6067 | GTACGA |
| 6068 | TGCGCA |
| 6069 | GTCGTA |
| 6070 | CGACTA |
| 6071 | CCGTAC |
| 6072 | CGATAC |
| 6073 | CGGTAC |
| 6074 | CGCACG |
| 6075 | GCGAAC |
| 6076 | CGCGCA |
| 6077 | AGCGCG |
| 6078 | ACGCGG |
| 6079 | ACGGCG |
| 6080 | GACGTC |
| 6081 | CGAGCG |
| 6082 | CGGACG |
| 6083 | ACGCCG |
| 6084 | ACGCGC |
| 6085 | GCGTAC |
| 6086 | CGCGAG |
| 6087 | CGCGGA |
| 6088 | CGCGTC |
| 6089 | CGGCGA |
| 6090 | CCGCGA |
| 6091 | CGCCGA |
| 6092 | ACCGGT |
| 6093 | CCGACG |
| 6094 | CCGTCG |
| 6095 | CGTCGC |
| 6096 | CGACGC |
| 6097 | ACGACG |
| 6098 | AACGCG |
| 6099 | ACGTCG |
| 6100 | CGCGAC |
| 6101 | CGAACG |
| 6102 | CGACCG |
| 6103 | ACGCGA |
| 6104 | CGCGAA |
| 6105 | CGACGA |
| 6106 | GTCGAC |
| 6107 | CGTCGA |
| 6108 | CGCGTA |
| 6109 | ACGCGT |
| 6110 | TCGCGA |
| 6111 | CGATCG |
| 6112 | CGTACG |

### (e) Linkers Between One or More Binding Segments on Donor DNAs

In some embodiments, a nucleotide sequence (a linker) is designed to be in between one or more binding segments on donor DNAs.

In some embodiments, the linker is about 1 to 30-nucleotide long. In other embodiments, the linker is about 10 to 25-nucleotide long. In some embodiments, the linker is at least 5-nucleotide long. In some embodiments, the linker is at least 10-nucleotide long. In some embodiments, the linker is at least 15-nucleotide long. In some embodiments, the linker is at least 20-nucleotide long. In some embodiments, the linker is at least 25-nucleotide long. In some embodiments, the linker is about 5-nucleotide long. In some embodiments, the linker is about 10-nucleotide long. In some embodiments, the linker is about 15-nucleotide long. In some embodiments, the linker is about 20-nucleotide long. In some embodiments, the linker is about 25-nucleotide long. In some embodiments, the linker is about 30-nucleotide long.

In some embodiments, the linker is a sequence of deoxyadenosines, deoxyguanosine, thymidine, deoxycytidines, or any combination thereof. In some specific embodiments, the linker is sequence of poly-deoxyadenosine. In other specific embodiments, the linker is sequence of poly-thymidine. In other specific embodiments, the linker is sequence of deoxyadenosines and thymidines.

### (f) Sequence of Interest (SOI)

In some embodiments, the SOI of the donor DNA as described herein comprises the target sequence with one or more nucleotide substitution, one or more nucleotide insertion, one or more nucleotide deletion, or any combination thereof.

In some specific embodiments, the SOI of the donor DNA as described herein comprises the target sequence with nucleotide insertion of 1 to 100 nucleotides. In other specific embodiments, the SOI of the donor DNA as described herein comprises the target sequence with nucleotide insertion of 1 to 50 nucleotides. In other specific embodiments, the SOI of the donor DNA as described herein comprises the target sequence with nucleotide insertion of 2 to 10 nucleotides. In other specific embodiments, the SOI of the donor DNA as described herein comprises the target sequence with nucleotide insertion of at least 2, at least 3, at least 4, at least 5, at least 6, at least 7, at least 8, at least 9, or at least 10 nucleotides. In other specific embodiments, the SOI of the donor DNA as described herein comprises the target sequence with nucleotide insertion of at least 11, at least 12, at least 13, at least 14, at least 15, at least 16, at least 17, at least 18, at least 19, or at least 20 nucleotides. In other specific embodiments, the SOI of the donor DNA as described herein comprises the target sequence with nucleotide insertion of at least 50, at least 100, at least 1,000, at least 10,000, or at least 100,000 nucleotides.

In some specific embodiments, the SOI of the donor DNA as described herein comprises the target sequence with nucleotide deletion of 1 to 50 nucleotides. In other specific embodiments, the SOI of the donor DNA as described herein comprises the target sequence with nucleotide deletion of 1 to 10 nucleotides. In other specific embodiments, the SOI of the donor DNA as described herein comprises the target sequence with nucleotide deletion of at least 2, at least 3, at least 4, at least 5, at least 6, at least 7, at least 8, at least 9, or at least 10 nucleotides. In other specific embodiments, the SOI of the donor DNA as described herein comprises the target sequence with nucleotide deletion of at least 11, at least 12, at least 13, at least 14, at least 15, at least 16, at least 17, at least 18, at least 19, or at least 20 nucleotides.

In some specific embodiments, the SOI of the donor DNA as described herein comprises the target sequence with nucleotide substitution of 1 to 50 nucleotides. In other specific embodiments, the SOI of the donor DNA as described herein comprises the target sequence with nucleotide substitution of 1 to 10 nucleotides. In other specific embodiments, the SOI of the donor DNA as described herein comprises the target sequence with nucleotide substitution of at least 2, at least 3, at least 4, at least 5, at least 6, at least 7, at least 8, at least 9, or at least 10 nucleotides. In other specific embodiments, the SOI of the donor DNA as described herein comprises the target sequence with nucleotide substitution of at least 11, at least 12, at least 13, at least 14, at least 15, at least 16, at least 17, at least 18, at least 19, or at least 20 nucleotides.

Furthermore, the SOI of the donor DNA as described herein serves as a template for homologous recombination after the guide RNA-medicated nuclease cut, so in some embodiments, the second portion of the donor DNA further comprises an upstream and/or a downstream homology arm.

In some embodiments, the upstream homology arm described herein is about 10 to 1,000-nucleotide long. In other embodiments, the upstream homology arm described herein is about 10 to 80-nucleotide long. In some embodiments, the upstream homology arm described herein is at least 10, at least 15, at least 20, at least 25, at least 30, at least 35, at least 40, at least 45, at least 50, at least 55, at least 60, at least 65, or at least 70 base long. In some specific embodiments, the upstream homology arm described herein is about 20-nucleotide long. In some specific embodiments, the upstream homology arm described herein is about 25-nucleotide long. In some specific embodiments, the upstream homology arm described herein is about 30-nucleotide long. In some specific embodiments, the upstream homology arm described herein is about 35-nucleotide long.

In some embodiments, the downstream homology arm described herein is about 10 to 1,000-nucleotide long. In other embodiments, the downstream homology arm described herein is about 10 to 80-nucleotide long. In some embodiments, the downstream homology arm described herein is at least 10, at least 15, at least 20, at least 25, at least 30, at least 35, at least 40, at least 45, at least 50, at least 55, at least 60, at least 65, or at least 70 base long. In some specific embodiments, the downstream homology arm described herein is about 20-nucleotide long. In some specific embodiments, the downstream homology arm described herein is about 25-nucleotide long. In some specific embodiments, the downstream homology arm described herein is about 30-nucleotide long. In some specific embodiments, the downstream homology arm described herein is about 35-nucleotide long.

In some embodiments, the second portion of the donor DNA further comprises a distal and/or a proximal homology arm. In some embodiments, the distal homology arm is about 10, 15, 20, 25, 30, 35, 40, 45, 50, 55, 60, 65, 70, 75, 80-nucleotide long. In some embodiments, the proximal homology arm is about 10, 15, 20, 25, 30, 35, 40, 45, 50, 55, 60, 65, 70, 75, 80-nucleotide long.

In some specific embodiments, the SOI of the donor DNA described herein is optimized to avoid any PAM sequences of the CRISPR nuclease used in the system. For example, in some embodiments, the SOI of the donor DNA described herein is optimized by a silent mutation to avoid any PAM sequences of the CRISPR nuclease used in the system while no change on the level of amino acid is introduced in the SOI.

### (g) Other Features of Donor DNAs

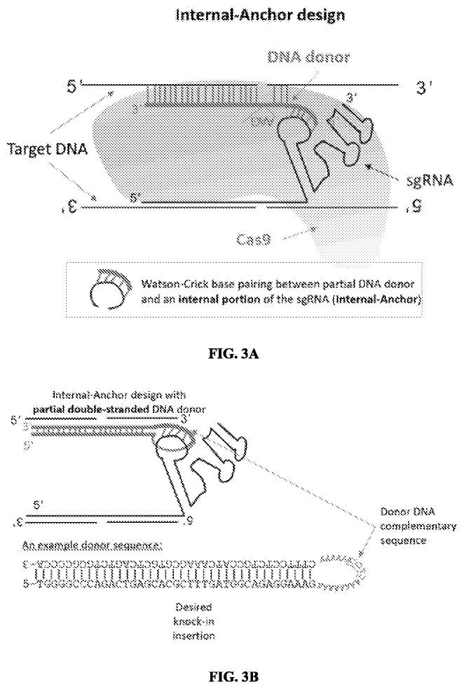

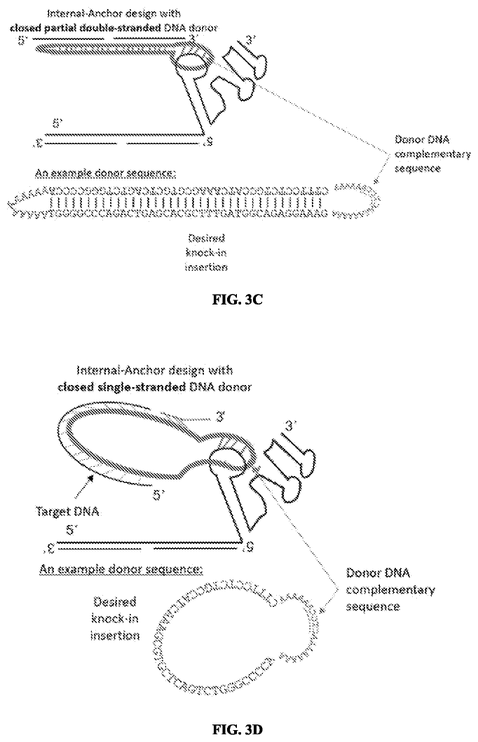

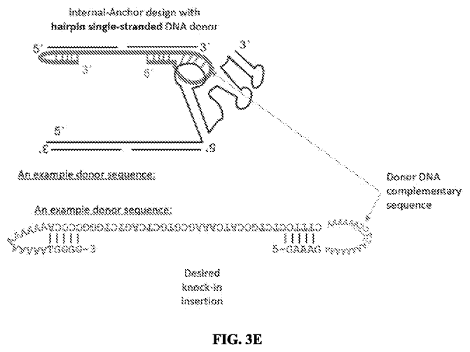

The donor DNA as described herein, as illustrated in FIG. 3A-FIG. 3E as non-limiting examples, can be in various design. In some embodiments, the donor DNA is single-stranded. In other embodiments, the first portion of the donor DNA is single-stranded and the second portion of the donor DNA is fully double-stranded. In other embodiments, the first portion of the donor DNA is single-stranded and the second portion of the donor DNA is partially double-stranded. In some embodiments, the donor DNA is close ended on its 3′ end. In some embodiments, the donor DNA is close ended on its 5′ end. In some embodiments, the donor DNA is close ended on its 3′ and 5′ ends. In some specific embodiments, the donor DNA forms a secondary structure of a hairpin, wherein the one or more binding segments are located within the loop of the hairpin. In some specific embodiments, the donor DNA forms a secondary structure with a dumbbell shape, wherein the one or more binding segments are located within one loop of the dumbbell. In some specific embodiments, the donor DNA described herein is a circular DNA. In some specific embodiments, the donor DNA described herein forms a secondary structure with a partial dumbbell shape, wherein the one or more binding segments are located within one loop of the dumbbell.

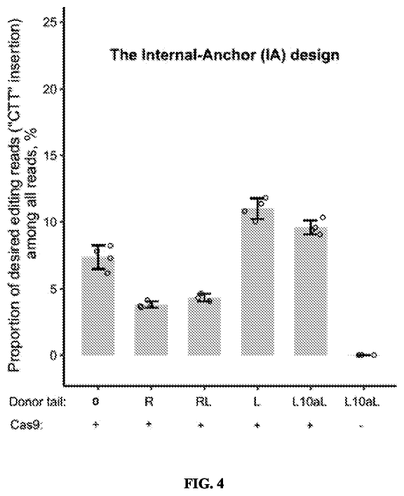

Furthermore, the first portion of the donor DNAs as described herein, which comprises one or more binding segments, can be designed to be various locations of donor DNAs, as tested in FIG. 4 and FIG. 5. In some embodiments, the first portion of the donor DNA is at 5′ of the second portion of the donor DNA. In other embodiments, the first portion of the donor DNA is at 3′ of the second portion of the donor DNA.

### (h) Cas Proteins

In some embodiments, the system described herein further comprises a CRISPR nuclease. In some specific embodiments, the CRISPR nuclease is a DNA nuclease.

In some embodiments, the CRISPR nuclease is a class I CRISPR nuclease. In other embodiments, the CRISPR nuclease is a class II CRISPR nuclease.

In some embodiments, the CRISPR nuclease is a type I CRISPR nuclease. In some specific embodiments, the CRISPR nuclease is a type I-A, type I-B, I-C, I-D, I-E, I-F, or I-U CRISPR nuclease. In other embodiments, the CRISPR nuclease is a type II CRISPR nuclease. In some specific embodiments, the CRISPR nuclease is a type II-A, type II-B, or type II-C CRISPR nuclease. In some embodiments, the CRISPR nuclease is a type III CRISPR nuclease. In some specific embodiments, the CRISPR nuclease is a type III-A, type III-B, type III-C, or type III-D CRISPR nuclease. In some embodiments, the CRISPR nuclease is a type IV CRISPR nuclease. In other embodiments, the CRISPR nuclease is a type V CRISPR nuclease. In some specific embodiments, the CRISPR nuclease is a type V-A, V-B, or V-C CRISPR nuclease.

In some embodiments, the DNA nuclease described herein is a Cas1, a Cas2, a Cas3, a Cas4, a Cas5, a Cas6, a Cas7, a Cas8, a Cas9, a Cas10, a Cas12, a Cas14, a CasΦ, a Casm, or a Cmr. In some specific embodiments, when the DNA nuclease is a Cas 9, the DNA nuclease is a high-fidelity Cas9 or an eCas9. In some specific embodiments, when the DNA nuclease is a Cas 12, the DNA nuclease is a Cas12a (Cpf1), a Cas12b, a Cas12c, a Cas12d, a Cas12e, a Cas12g, a Cas12h, a Cas12i, a Cas12j, or a Cas12k.

In one aspect, provided herein is a kit comprising the donor DNA described herein and the modified sgRNA described herein. In some embodiments, the kit comprises the donor DNA described herein, the modified sgRNA described herein, and the CRISPR nuclease described herein.

In one aspect, provided herein is a pharmaceutical composition comprising the donor DNA described herein, the modified sgRNA described herein, and a pharmaceutically acceptable salt or derivative thereof. In some embodiments, the kit comprises the donor

DNA described herein, the modified sgRNA described herein, the CRISPR nuclease described herein, and a pharmaceutically acceptable salt or derivative thereof.

### 2. Methods of Using the Systems Described Herein

In one aspect, provided herein is a method of modifying a cell, wherein the method comprises transporting the system as described herein into the cell.

In some embodiments, the transporting comprises (a) incubating the CRISPR nuclease described herein and the modified sgRNA described herein to form a ribonucleoprotein (RNP) complex; (b) applying the donor DNA to the RNP complex; and (c) delivering the RNP complex-donor DNA from (b) to the cell. In some specific embodiments, the RNP complex is formed in vitro in step (a). In some embodiments, the CRISPR nuclease described herein is expressed and purified using relevant plasmid. In some embodiments, the modified sgRNA is in vitro transcribed from a corresponding ssDNA. In some non-limiting exemplary embodiments, the ratio of the CRISPR nuclease and the modified sgRNA in step (a) is about 1:0.5 to about 1:10. In other non-limiting exemplary embodiments, the ratio of the CRISPR nuclease and the modified sgRNA in step (a) is about 1:1 to about 1:1.2. In other non-limiting exemplary embodiments, the ratio of the CRISPR nuclease and the modified sgRNA in step (a) is about 1:0.6, about 1:1.2, about 1:2, or about 1:5. In some specific embodiments, applying the donor DNA to the RNP complex is carried out in vitro in step (b). In some specific embodiments, delivering the RNP complex-donor DNA from (b) to the cell is achieved by viral vectors, liposomes, and/or lipid nanoparticles. In other specific embodiments, delivering the RNP complex-donor DNA from (b) to the cell is achieved by electroporation. In one embodiment, delivering the RNP complex-donor DNA from (b) to the cell is achieved by nucleofection (see e.g., Distler et al., Exp Dermatol 2005 April; 14 (4): 315-20). In other specific embodiments, delivering the RNP complex-donor DNA from (b) to the cell is achieved by a polyethylene glycol (PEG) mediated transfection. In other specific embodiments, delivering the RNP complex-donor DNA from (b) to the cell is achieved by a gene gun.

In some embodiments, the transporting comprises (a) providing one or more vectors comprising a nucleotide sequence encoding the CRISPR nuclease described herein and a nucleotide sequence encoding the modified gRNA described herein; (b) delivering the one or more vectors of (a) to the cell; and (c) delivering the donor DNA described herein to the cell. In some preferred embodiments, step (c) is performed about 6 to 48 hours after step (b). In other preferred embodiments, step (c) is performed at least 6 hours after step (b). In other preferred embodiments, step (c) is performed at least 12 hours after step (b). In other preferred embodiments, step (c) is performed at least 18 hours after step (b). In other preferred embodiments, step (c) is performed at least 24 hours after step (b). In other preferred embodiments, step (c) is performed at least 30 hours after step (b). In other preferred embodiments, step (c) is performed at least 36 hours after step (b). In other preferred embodiments, step (c) is performed at least 42 hours after step (b). In other preferred embodiments, step (c) is performed at least 48 hours after step (b). In some specific embodiments, the delivering of the one or more vectors of (a) to the cell is achieved by viral vectors, liposomes, and/or lipid nanoparticles. In some specific embodiments, the delivering of the donor DNA to the cell is achieved by viral vectors, liposomes, and/or lipid nanoparticles.

In some embodiments, the cell being modified herein is an immune cell. In some specific embodiments, the cell being modified herein is a T cell. In some specific embodiments, the cell being modified herein is a B cell. In some specific embodiments, the cell being modified herein is an NK cell. In some specific embodiments, the cell being modified herein is a hematopoietic stem cell.

In some embodiments, method described herein is performed in vitro. In other embodiments, method described herein is performed ex vivo. In other embodiments, method described herein is performed in vivo.

Since the cleavage efficiency of the nuclease described herein is relatively high and the efficiency of homology directed repair (HDR) is relatively low, a large portion of the nuclease-induced double stranded breaks (DSBs) might be repaired via NHEJ. In other words, the resulting population of cells might contain some combination of wild type alleles, NHEJ-repaired alleles, and/or the desired edited allele based on the design of the SOI of the donor DNA described herein. The method of modifying the cell using the system described herein is advantageous, in view of a higher percentage of desired editing, low off-target rate, and/or low translocation large insertion, or large deletion rate.

In some embodiments, a percentage of desired editing using the method described herein is at least 10%, at least 50%, at least 100%, or at least 200% higher than a comparable system without the donor DNA comprising a first portion that binds to the modified sgRNA and/or without the modified sgRNA. In other embodiments, the method described herein has an off-target rate at least 10%, at least 50%, or at least 100% lower than a comparable system without the donor DNA comprising a first portion that binds to the modified sgRNA and/or without the modified sgRNA. In other embodiments, the method has a translocation, large insertion, or large deletion rate at least 10%, at least 50%, or at least 100% lower than a comparable system without the donor DNA comprising a first portion that binds to the modified sgRNA and/or without the modified sgRNA.

In order to further increase the HDR rate, in some embodiments, the method described herein further comprises synchronizing the cell to S phase. In other embodiments, the method described herein further comprises synchronizing the cell to G2 phase. In other embodiments, the method described herein further comprises inhibiting genes involved in NHEJ pathway. In other embodiments, the method described herein further comprises inhibiting genes involved in mismatch-repair pathway. In other embodiments, the method described herein further comprises fusing ctIP, a protein involved in double-stranded break resection, to the CRISPR nuclease. In other embodiments, the method described herein further comprises fusing single-strand annealing protein to the CRISPR nuclease.

In another aspect, provided herein is a method of treating a genetic disorder, wherein the method comprises administering to a subject with an effective amount of the system described herein. In some embodiments, the SOI comprises a sequence that reverses or alleviate the genetic disorder.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. Efforts have been made to ensure accuracy with respect to numbers used (e.g., amounts, percentages, etc.), but some experimental errors and deviations should be accounted for

### Example 1: “Fusion-Oligo Design” and “Terminal Anchor Design” were Proven to be Dysfunctional

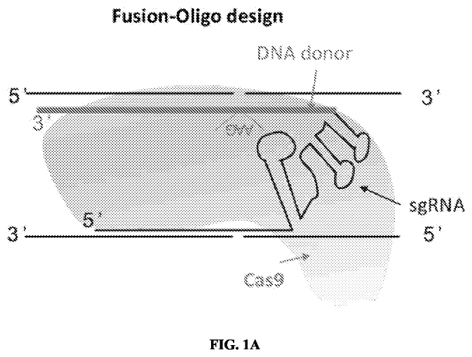

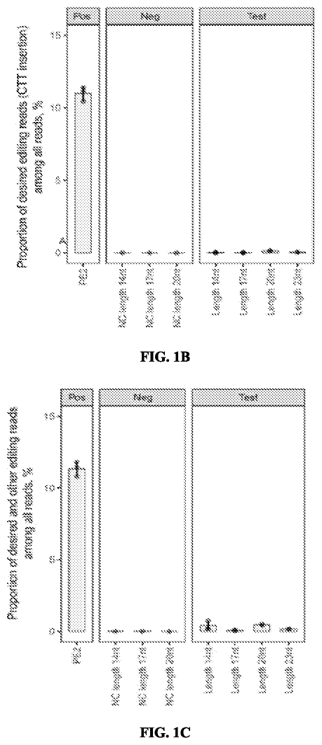

The “Fusion-Oligo design” is referred to as a system where a donor DNA is fused to a guide RNA (see FIG. 1A). Briefly, guide RNA-donor DNA fusions with various lengths of the donor DNA, listed in Table 5 below, were tested. As shown in FIG. 1B and FIG. 1C, the “Fusion-Oligo design” showed negligible editing efficiency compared to positive control.

TABLE 5
Sequences of guide RNA-donor DNA fusions

| SEQ ID | | |
|---|---|---|
| NO. | Sequence | Note |
| 6113 | rArGrCrArUrArGrCrArA | “Length 14nt” |
| | rGrUrUrArArArArUrArA | in FIG. 1B/1C |
| | rGrGrCrUrArGrUrCrCrG | |
| | rUrUrArUrCrArArCrUrU | |
| | rGrArArArArArGrUrGrG | |
| | rCrArCrCrGrArGrUrCrG | |
| | rGrUrGrCrUrUrUctttcc | |
| | tctgccatcaaagcgtgctc | |
| | agtctgg | |
| 6114 | rArGrCrArUrArGrCrArA | “Length 17 nt” |
| | rGrUrUrArArArArUrArA | in FIG. 1B/1C |
| | rGrGrCrUrArGrUrCrCrG | |
| | rUrUrArUrCrArArCrUrU | |
| | rGrArArArArArGrUrGrG | |
| | rCrArCrCrGrArGrUrCrG | |
| | rGrUrGrCrUrUrUctttcc | |
| | tctgccatcaaagcgtgctc | |
| | agtctgggcc | |
| 6115 | rArGrCrArUrArGrCrArA | “Length 20 nt” |
| | rGrUrUrArArArArUrArA | in FIG. 1B/1C |
| | rGrGrCrUrArGrUrCrCrG | |
| | rUrUrArUrCrArArCrUrU | |
| | rGrArArArArArGrUrGrG | |
| | rCrArCrCrGrArGrUrCrG | |
| | rGrUrGrCrUrUrUctttcc | |
| | tctgccatcaaagcgtgctc | |
| | agtctgggcccca | |
| 6116 | rArGrCrArUrArGrCrArA | “Length 23 nt” |
| | rGrUrUrArArArArUrArA | in FIG. 1B/1C |
| | rGrGrCrUrArGrUrCrCrG | |
| | rUrUrArUrCrArArCrUrU | |
| | rGrArArArArArGrUrGrG | |
| | rCrArCrCrGrArGrUrCrG | |
| | rGrUrGrCrUrUrUctttcc | |
| | tctgccatcaaagcgtgctc | |
| | agtctgggccccaagg | |

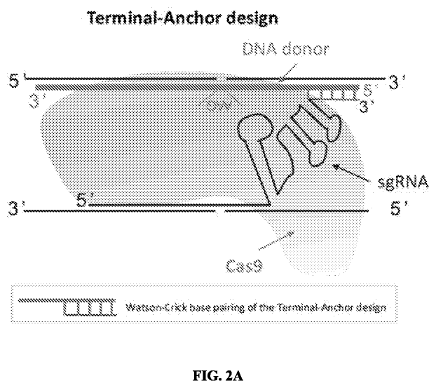

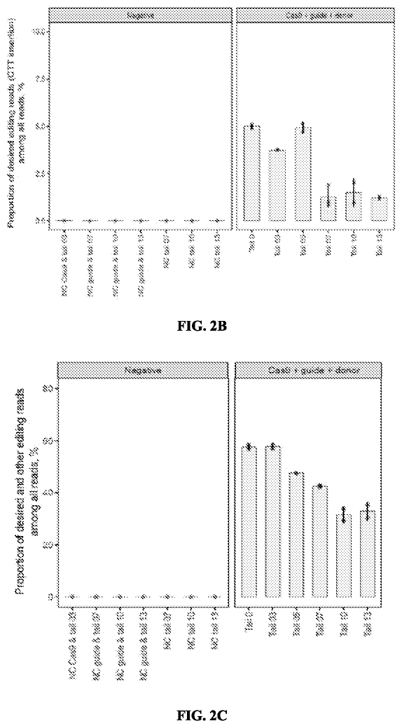

TABLE 6
Donor DNA Sequences in “Terminal Anchor design”

| SEQ ID | | |
|---|---|---|
| NO. | Sequence | Note |
| 6117 | C*T*TTCCTC | “Tail 0” in FIG. |
| | TGCCATCAAA | 2B/2C |
| | GCGTGCTCAG | |
| | TCTGGGCCC* | |
| | C*A | |
| 6118 | C*C*ACTTTC | “Tail 03” in |
| | CTCTGCCATC | FIG. 2B/2C |
| | AAAGCGTGCT | |
| | CAGTCTGGGC | |
| | CC*C*A | |
| 6119 | C*T*CCACTT | “Tail 05” in |
| | TCCTCTGCCA | FIG. 2B/2C |
| | TCAAAGCGTG | |
| | CTCAGTCTGG | |
| | GCCC*C*A | |
| 6120 | T*C*CTCCAC | “Tail 07” in |
| | TTTCCTCTGC | FIG. 2B/2C |
| | CATCAAAGCG | |
| | TGCTCAGTCT | |
| | GGGCCC*C*A | |
| 6121 | G*T*GTCCTC | “Tail 10” in |
| | CACTTTCCTC | FIG. 2B/2C |
| | TGCCATCAAA | |
| | GCGTGCTCAG | |
| | TCTGGGCCC* | |
| | C*A | |
| 6122 | G*T*CGTGTC | “Tail 13” in |
| | CTCCACTTTC | FIG. 2B/2C |
| | CTCTGCCATC | |
| | AAAGCGTGCT | |
| | CAGTCTGGGC | |
| | CC*C*A | |

*denotes a phosphorothioate linkage

Separately, “Terminal-Anchor design” was also tested. The “Terminal-Anchor design” is referred to as a system which comprises a guide RNA that is partially complementary to a donor DNA, and the complementary sequence is located at a terminus of the guide RNA (see FIG. 2A). Briefly, donor DNAs with different lengths of complementary sequences to the 3′ end of guide RNA, listed in Table 6 above, were tested. As shown in FIG. 2B and FIG. 2C, the “Fusion-Oligo design” did not improve the desired editing efficiency.

Based on the above surprising and unexpected observations, it was concluded that the type and the location of the connection between donor DNAs and guide RNAs substantially affect the editing efficiency. Donor DNA being fused with or hybridized to termini of guide RNAs did not result in satisfactory editing efficiency.

### Example 2: Various Designs of Donor DNAs and Guide RNAs for “Internal Anchor Design”

The “Internal-Anchor design” is referred to as a system which comprises a guide RNA that is partially complementary to a donor DNA, and the complementary sequence is located at the internal part of the guide RNA (see FIG. 3A). Various different donor constructs were also designed (see FIGS. 3B-3E).

With the Internal-Anchor CRISPR system, the effect of different positions of the fragments of donor DNA (“tail”) that was complementary to the guide RNA on the efficiencies of desired gene editing (‘CTT’ insertion at the target locus) in HEK293T cells was first evaluated. Different DNA donor designs included: conventional design where a donor DNA is without tails (indicated as “0” in FIG. 4), the DNA sequence that is complementary to the guide-RNA is at the 3′ end of the donor DNA (indicated as “R” in FIG. 4), one DNA tail is at the 3′ end and a second ‘tail’ is at the 5′ end of the donor DNA (indicated as “RL” in FIG. 4), one DNA tail is at the 5′ end of the donor DNA (indicated as “L” in FIG. 4), and the “L” design is followed by ten deoxyadenosines and a second ‘L’ (indicated as “L10aL” in FIG. 4). One condition without adding Cas9 was included as a negative control (last column in FIG. 4). The results showed that the “R” and “RL” designs showed lower efficiencies of desired editing compared with the conventional DNA donor. In contrast, the “L” and “L10aL” showed statistically significant higher efficiencies of desired editing when compared with the conventional DNA donor (see FIG. 4).

Next, the effect of a tail sequence on the donor DNA, matching or non-matching with the internal anchor (IA) sequence of the guide RNA, on editing efficiency was tested. As expected, negative control samples (no Guide RNAs, no Donor DNAs, or no Cas9, shown as “-” under “guide,” “Donor tail,” or “Cas9” categories) showed no observable CTT insertion (see FIG. 5). Furthermore, the three other conditions: (1) WT guide RNA without the IA sequence and a donor without tail (“Guide: WT” and “Donor tail: 0”); (2) a WT guide RNA without the IA sequence and a donor with a mock tail (“Guide: WT” and “Donor tail: nonM”); and (3) a guide RNA with the IA sequence and a non-matching donor tail (“Guide: IA” and “Donor tail: nonM”), which contains a mock 5-nucleotide sequence that was not matching with the guide IA, showed similar CTT insertion efficiencies of about 5%-6%. In contrast, a guide RNA with the IA sequence together with a donor DNA of a tail design of either “L” or “L10aL” (“Guide: IA” and “Donor tail: L” and “Guide: IA” and “Donor tail: L10aL”) both increased the CTT insertion efficiencies by about 2-fold (see FIG. 5).

### Example 3: Ratio of Guide RNA and Cas9

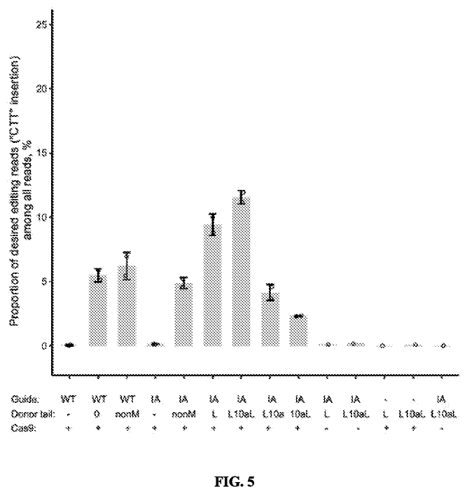

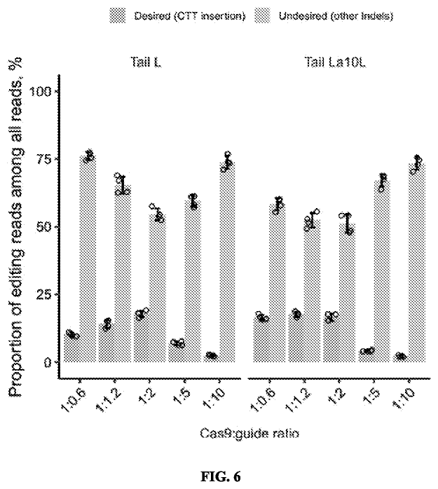

Different ratios of the two (ranging from 1:0.6 to 1:10) were tested using two tail designs (“L” and “L10aL,” similarly labeled as in Example 1). As shown in FIG. 6, the efficiencies of the desired (′CTT′ insertion at the target locus in this non-limiting example) and undesired (other indels) gene editing showed that the 1:1.2 and 1:2 ratios had the highest efficiencies compared with other ratios. Higher amounts of RNA guides (1:5 or 1:10 ratio), however, resulted in significantly reduced efficiencies of desired editing, whereas other indels, such as the ones caused by the non-homologous DNA end joining (NHEJ) pathway, were not affected (see FIG. 6). Therefore, the relative amounts of Cas9 to the guide RNA affected editing efficiency.

### Example 4: The Length of Inner Link Between Two Binding Segments on Donor DNA

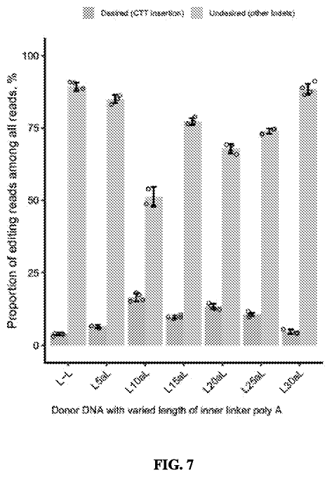

The effect of the tail-tail inner sequence length (0, 5, 10, 15, 20, 25, and 30 deoxyadenosines; FIG. 7) of the donor DNA on the efficiencies of desired (‘CTT’ insertion at the target locus) and undesired (other indels) gene editing was examined, using the Internal-Anchor CRISPR system in HEK293T cells. A length of ten deoxyadenosines (“L10aL”) showed the highest desired CTT insertion than donor DNA with other lengths. Correspondingly, the undesired other indels proportion was the lowest in the L10aL design among all, indicating that the undesired editing byproducts via NHEJ pathway were substantially suppressed by the L10aL design.

### Example 5: The Length of Homology Arms

The effect of the length of DNA donor homology arms on HDR editing efficiency was studied. Donors of different lengths at both distal (D) and proximal (P) sides relative to the cutting position were tested as shown in FIG. 8. Both short (<20 bases) and long (>36 bases) homology arms showed lower CTT insertion efficiencies when compared with those donors having the median lengths (20 to 36 bases). For the donors with D20P30 and D20P36 homology arms, the proportion of CTT insertion reads were higher than the number of undesired indels reads in the same samples.

### Example 6: Rate of Off-Target, Translocation, Large Insertion, and Large Deletion

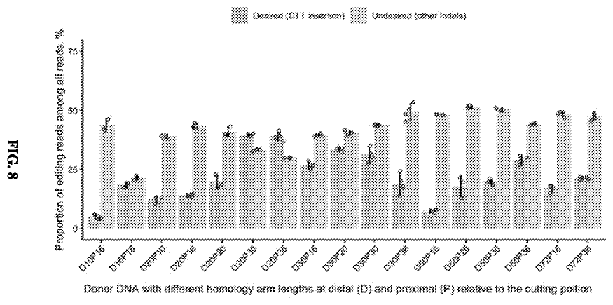

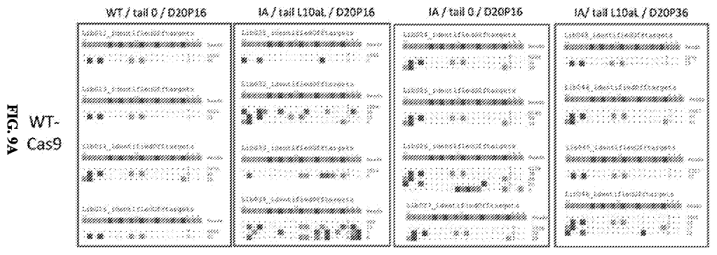

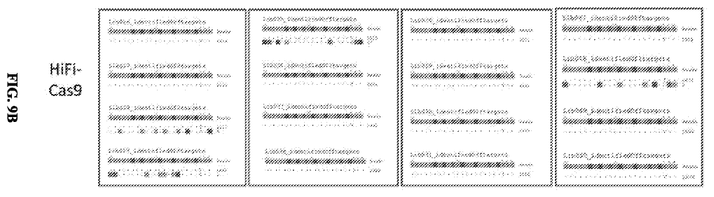

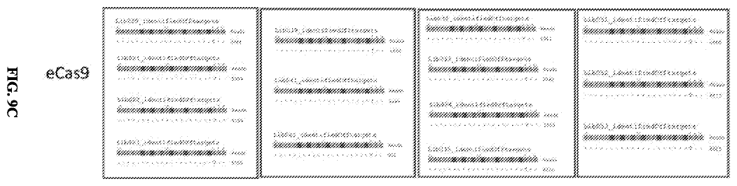

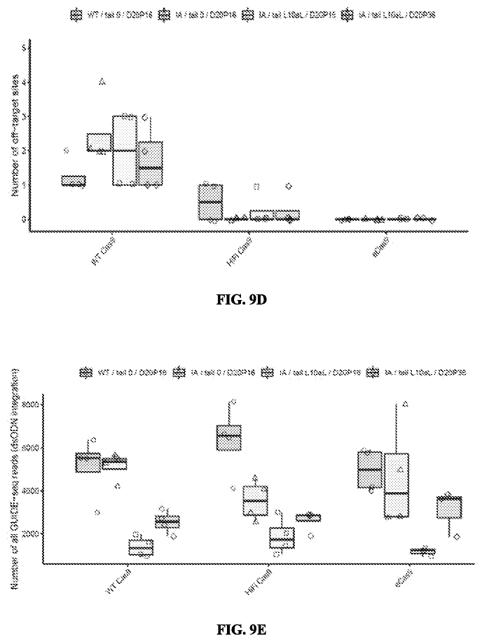

Genome-wide off-target profiling was evaluated when targeting the HEK3 site using the Internal-Anchor CRISPR system with various elements: (1) the different guides (“WT” denotes guide RNA without internal anchors, and “IA” denotes guide RNA with internal anchors); (2) donor DNA tails (“0” denotes donor DNA without tails/binding segments that are complementary to the guide RNA, and “L10aL” denotes two DNA tails/binding segments are at the 5′ end of the donor DNA, with ten deoxyadenosines in between); (3) homology arms (“D20P16” denotes donor DNAs have a 20-nucleotide long distal homology arm and a 16-nucleotide long proximal arm; “D20P36” denotes donor DNAs have a 20-nucleotide long distal homology arm and a 36-nucleotide long proximal arm); and (4) by three SpCas9 (WT, HiFi and eCas9). As expected, the high-fidelity Cas9s showed reduced off-target site numbers (see FIG. 9B and FIG. 9D), indicating that the Internal-Anchor CRISPR system is compatible with the high-fidelity Cas nucleases. Interestingly, the numbers of total GUIDE-seq reads were lower when using the Internal-Anchor CRISPR system as compared with normal CRISPR system (see FIG. 9E). Because the number of total GUIDE-seq reads is correlated with NHEJ efficiency during the integration of a dsODN with target double strand breaks (DSBs), as part of the GUIDE-seq protocol, the results indicate that the Internal-Anchor CRISPR system largely suppressed the undesired NHEJ pathway.

### Example 7: Long-Fragment Deletion in HEK3

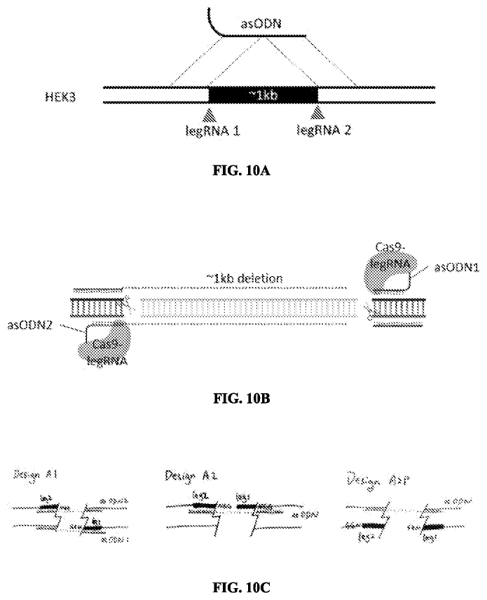

To delete a long-fragment in HEK3, dual modified sgRNAs (i.e., leg1 and leg2 in FIG. 10C) comprising an internal anchor (SEQ ID.: legRNA_Int_Anc) in the loop on top of the upper stem located in the first hairpin (the modified sgRNAs also termed as “lead editing guide RNAs”, legRNAs in short) were designed to target 2 different loci 991-bp apart on HEK3 (FIG. 10A), together with SpCas9 and anchored ssDNA (asODN) donor template consisting of 36-base homology arms upstream and downstream of the deletion site and a legRNA anchored tail (FIG. 10B). Three asODN-legRNA approaches were designed and tested (FIG. 10C), Design A1: asODN1 (SEQ ID.: HEK3_A1_asODN1), asODN2 (SEQ ID.: HEK3_A1_asODN2), legRNA1 (SEQ ID: A1_HEK3_legRNA1) and legRNA2 (SEQ ID: A1_HEK3_legRNA2) targeting different strands; Design A2: asODN3 (SEQ ID: HEK3_A2_asODN) and both legRNA3 (SEQ ID: A2_HEK3_legRNA1) and legRNA4 (SEQ ID: A2_HEK3_legRNA2) targeting the same upper strand; Design A2p: both legRNA5 (SEQ ID: A2p_HEK3_legRNA1) and legRNA6 (SEQ ID: A2p_HEK3_legRNA2) targeting the same lower strand, while the asODN4 (SEQ ID: HEK3_A2p_asODN) targeting the other strand.

### Experiment Procedure

LegRNA production. The legRNAs were produced by in vitro transcription (IVT). Briefly, DNA template of the legRNA was constructed and amplified by PCR. Then, T7 polymerase was used to transcribe the DNA to RNA, followed by DNAse I treatment to remove the remaining DNA template in the sample.

Cas9-legRNA ribonucleoprotein (RNP) transfection. HEK293T cells were seeded in a 24-well plate one day before transfection. Firstly, legRNAs were assembled with cas9 protein to form a ribonucleoprotein complex (RNP). Next, asODN were added to form a RNP-asODN complex. The RNP-asODN complex were transfected into the cells using lipofectamine. DNA from the cells were extracted 2-3 days after transfection.

PCR and gel electrophoresis. PCR and gel electrophoresis were used to visualize the 1 kb deletion. 1 forward primer and 2 reverse primers were used in the PCR reaction so that the WT product and the deletion product can be amplified and visualized on a gel simultaneously.

Amplicon sequencing. The amplicon sequencing library was generated with 2-step PCR using target site specific primers and index primers. The library was sequenced by illumine Nextseq500 or iSeq sequencing system.

### Results

The results of using different asODN-legRNA designs were shown in FIGS. 11A-11C. The Design A1 yielded the deletion product at about 30% of the WT product, i.e., about 23% (=30%/(100%+30%)) deletion efficiency (FIG. 11A and FIG. 11B), the deletion was confirmed by sequencing (FIG. 11C).

### Example 8: Long-Fragment Deletion in HPRT1

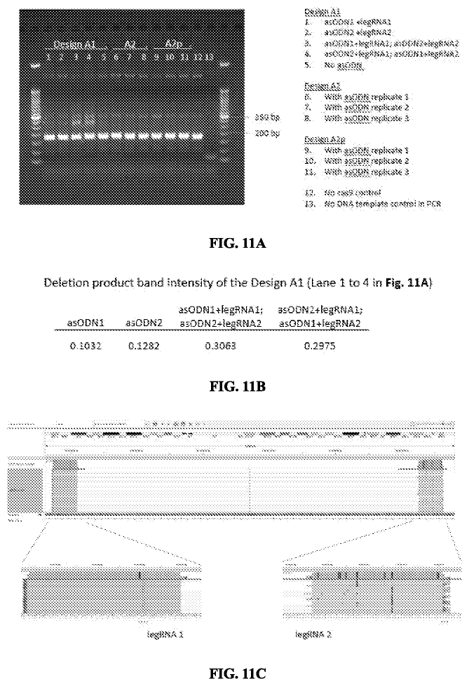

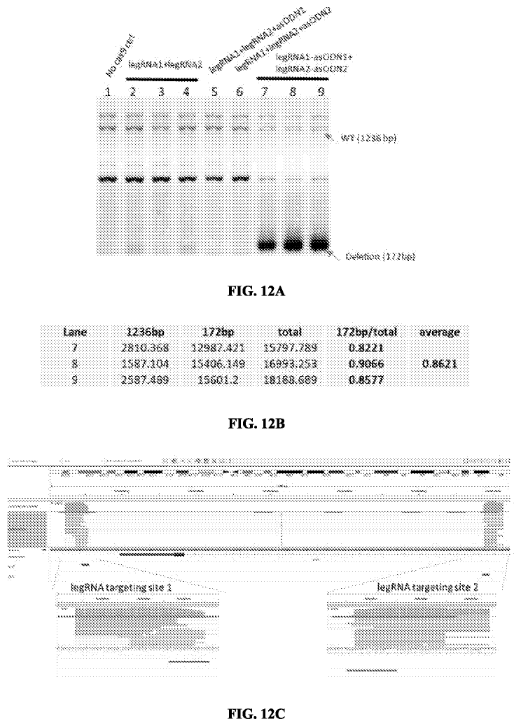

Similarly, as Example 1, a long-fragment (1075-bp) deletion in HPRT1 was tested. A similar dual asODN-legRNA system as Design A1 was used (asODN5: SEQ ID: HPRT_A1_asODN1; asODN6: SEQ ID: HPRT_A1_asODN2; legRNA7: SEQ ID: HPRT_legRNA1; and legRNA8: SEQ ID: HPRT_legRNA2), and the results were shown in FIGS. 12A-12C. The dual asODN-legRNA showed deletion efficiency approximately up to 86% (FIG. 12A and FIG. 12B), though the deletion efficiency analyzed by gel intensity might be overestimated because amplification is favored to short amplicon than long amplicon. The deletion was confirmed by sequencing (FIG. 12C).

### Example 9: Large-Fragment Insertion in GAPDH

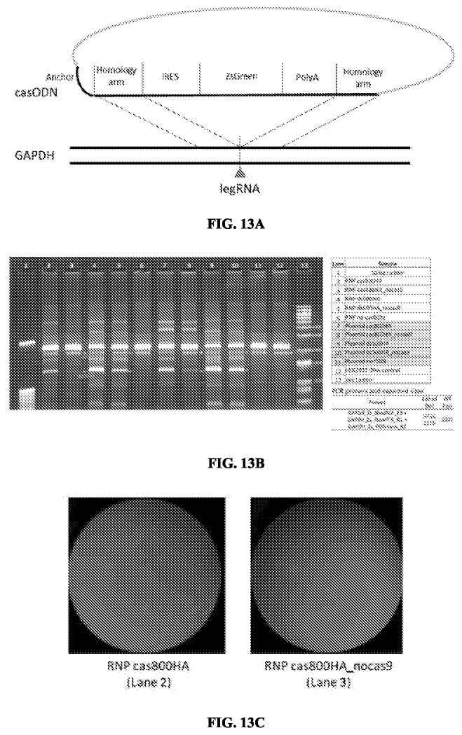

To test large-fragment insertion of 1734 bases into GAPDH, cas9 and legRNA (SEQ ID: GAPDH_legRNA) with a circularized anchored-ssODN (casODN), cas800HA (SEQ ID: casODN_800HA, internal anchor sequence: ACATTGTTCTCACTT (SEQ ID NO: 6123) were used (FIG. 13A). The casODN serves as the insertion template containing an anchor sequence, left homology arm, IRES, a green fluorescent protein (ZsGreen) encoding sequence, polyA, and right homology arm (FIG. 13A). Similar transfection experiment conditions as Example 1 were used, except that two forms of cas9 were used, ribonucleotide (RNP) and plasmid (for comparison). Regular double-strand DNA templates (dsONA), ds500HA (SEQ ID: dsODN_500HA) were also included for comparison. The results showed that the RNP-casODN treatment yielded much stronger insertion product than no-cas9 control (FIG. 13B). The low-level product in the no-cas9 control (FIG. 13B Lane 3) could be due to the classical homology directed recombination. When using double-strand temples, insertions of the 1734 bases were also observed but their no-cas control also showed substantial insertion (FIG. 13B Lanes 4 & 5). When cas9 were expressed by plasmid, similar results as RNP were observed (FIG. 13B Lanes 7-12). The edited cells from the RNP-casODN treatment showed clear functional insertion of the ZsGreen protein in the treated cells, while the no-cas9 control cells did not show noticeable green signal (FIG. 13C).

TABLE 7
Nucleic acid sequences used in Examples 7-9

| | |
|---|---|
| legRNA | UGGAG |
| Int_Anc | |
| A1_H | GGCCCAGACUGAGCACGUGAGUUUUAGAGCUAGUG |
| EK3_leg | GAGAAAUAGCAAGUUAAAAUAAGGCUAGUCCGUUA |
| RNA1 | UCAACUUGAAAAAGUGGCACCGAGUCGGUGC |
| | (SEQ ID NO: 6124) |
| A1_H | UGAUCACCUGCCCAAAUGUGGUUUUAGAGCUAGUG |
| EK3_leg | GAGAAAUAGCAAGUUAAAAUAAGGCUAGUCCGUUA |
| RNA2 | UCAACUUGAAAAAGUGGCACCGAGUCGGUGC |
| | (SEQ ID NO: 6125) |
| A2_H | CUGCCAUCACGUGCUCAGUCGUUUUAGAGCUAGUG |
| EK3_leg | GAGAAAUAGCAAGUUAAAAUAAGGCUAGUCCGUUA |
| RNA1 | UCAACUUGAAAAAGUGGCACCGAGUCGGUGC |
| | (SEQ ID NO: 6126) |
| A2_H | UGAUCACCUGCCCAAAUGUGGUUUUAGAGCUAGUG |
| EK3_leg | GAGAAAUAGCAAGUUAAAAUAAGGCUAGUCCGUUA |
| RNA2 | UCAACUUGAAAAAGUGGCACCGAGUCGGUGC |
| | (SEQ ID NO: 6125) |
| A2p | GGCCCAGACUGAGCACGUGAGUUUUAGAGCUAGUG |
| HEK3_le | GAGAAAUAGCAAGUUAAAAUAAGGCUAGUCCGUUA |
| gRNA1 | UCAACUUGAAAAAGUGGCACCGAGUCGGUGC |
| | (SEQ ID NO: 6124) |
| A2p | UGGGCAGGUGAUCAATGCUGUUUUAGAGCUAGUGG |
| HEK3_le | AGAAAUAGCAAGUUAAAAUAAGGCUAGUCCGUUAU |
| gRNA2 | CAACUUGAAAAAGUGGCACCGAGUCGGUGC |
| | (SEQ ID NO: 6127) |
| HEK3 | c*t*ccaGGAGATGCTCCCTAAGCATTGATCACCT |
| A1_as | GCCCAAATCGTGCTCAGTCTGGGCCCCAAGGATTG |
| ODN1 | ACCCAGG*C*C |
| | (SEQ ID NO: 6128) |
| HEK3 | c*t*ccaGGCCTGGGTCAATCCTTGGGGCCCAGAC |
| _A1_as | TGAGCACGATTTGGGCAGGTGATCAATGCTTAGGG |
| ODN2 | AGCATCT*C*C |
| | (SEQ ID NO: 6129) |
| HEK3_ | c*t*ccaCCAGCCCTGGCCTGGGTCAATCCTTGGG |
| A2_as | GCCCAGACATTTGGGCAGGTGATCAATGCTTAGGG |
| ODN | AGCATCT*C*C |
| | (SEQ ID NO: 6130) |
| HEK3_A | c*t*ccaCAAATCCTCGCATTTGGGCAGGAGATGC |
| 2p_as | TCCCTAAGCGTGCTCAGTCTGGGCCCCAAGGATTG |
| ODN | ACCCAGG*C*C |
| | (SEQ ID NO: 6131) |
| HPRT1 | |
| HPRT_le | GCCUGCAAACUGGUAGGCGCGUUUUAGAGCUAGUG |
| gRNA1 | GAGAAAUAGCAAGUUAAAAUAAGGCUAGUCCGUUA |
| | UCAACUUGAAAAAGUGGCACCGAGUCGGUGC |
| | (SEQ ID NO: 6132) |
| HPRT | AAGCAUGAUCAGAACGGUUGGUUUUAGAGCUAGUG |
| legRNA | GAGAAAUAGCAAGUUAAAAUAAGGCUAGUCCGUUA |
| 2 | UCAACUUGAAAAAGUGGCACCGAGUCGGUGC |
| | (SEQ ID NO: 6133) |
| HPRT | C*T*CCAGGGTTTTGACTATTTTAGCAAGCATGAT |
| A1_as | CAGAACGGCCTACCAGTTTGCAGGCTCACTAGGTA |
| ODN1 | GCCGTGG*G*A |
| | (SEQ ID NO: 6134) |
| HPRT | C*T*CCATCCCACGGCTACCTAGTGAGCCTGCAAA |
| A1_as | CTGGTAGGCCGTTCTGATCATGCTTGCTAAAATAG |
| ODN2 | TCAAAAC*C*C |
| | (SEQ ID NO: 6135) |
| GAP | AGCCCCAGCAAGAGCACAAGGUUUUAGAGCUAGAA |
| DH_leg | GUGAGAACAAUGUAAAUAGCAAGUUAAAAUAAGGC |
| RNA | UAGUCCGUUAUCAACUUGAAAAAGUGGCACC |
| | GAGUCGGUGC |
| | (SEQ ID NO: 6136) |
| casO | ACATTGTTCTCACTTgggaatgggactgaggctcc |
| DN_80 | cacctttctcatccaagactggctcctccctgccg |
| OHA | gggctgcgtgcaaccctggggttgggggttctggg |
| | gactggctttcccataatttcctttcaaggtgggg |
| | agggaggtagaggggtgatgtggggagtacgctgc |
| | agggcctcactccttttgcagaccacagtccatgc |
| | catcactgccacccagaagactgtggatggcccct |
| | ccgggaaactgtggcgtgatggccgcggggctctc |
| | cagaacatcatccctgcctctactggcgctgccaa |
| | ggctgtgggcaaggtcatccctgagctgaacggga |
| | agctcactggcatggccttccgtgtccccactgcc |
| | aacgtgtcagtggtggacctgacctgccgtctaga |
| | aaaacctgccaaatatgatgacatcaagaaggtgg |
| | tgaagcaggcgtcggagggccccctcaagggcatc |
| | ctgggctacactgagcaccaggtggtctcctctga |
| | cttcaacagcgacacccactcctccacctttgacg |
| | ctggggctggcattgccctcaacgaccactttgtc |
| | aagctcatttcctggtatgtggctggggccagaga |
| | ctggctcttaaaaagtgcagggtctggcgccctct |
| | ggtggctggctcagaaaaagggccctgacaactct |
| | tttcatcttctaggtatgacaacgaatttggctac |
| | agcaacagggtggtggacctcatggcccacatggc |
| | ctccaaggagtaagacccctggaccaccagcccca |
| | gcaagagcacacgttactggccgaagccgcttgga |
| | ataaggccggtgtgcgtttgtctatatgttatttt |
| | ccaccatattgccgtcttttggcaatgtgagggcc |
| | cggaaacctggccctgtcttcttgacgagcattcc |
| | taggggtctttcccctctcgccaaaggaatgcaag |
| | gtctgttgaatgtcgtgaaggaagcagttcctctg |
| | gaagcttcttgaagacaaacaacgtctgtagcgac |
| | cctttgcaggcagcggaaccccccacctggcgaca |
| | ggtgcctctgcggccaaaagccacgtgtataagat |
| | acacctgcaaaggcggcacaaccccagtgccacgt |
| | tgtgagttggatagttgtggaaagagtcaaatggc |
| | tctcctcaagcgtattcaacaaggggctgaaggat |
| | gcccagaaggtaccccattgtatgggatctgatct |
| | ggggcctcggtgcacatgctttacatgtgtttagt |
| | cgaggttaaaaaaacgtctaggccccccgaaccac |
| | ggggacgtggttttcctttgaaaaacacgatgata |
| | ataatggcccagtccaagcacggcctgaccaagga |
| | gatgaccatgaagtaccgcatggagggctgcgtgg |
| | acggccacaagttcgtgatcaccggcgagggcatc |
| | ggctaccccttcaagggcaagcaggccatcaacct |
| | gtgcgtggtggagggcggccccttgcccttcgccg |
| | aggacatcttgtccgccgccttcatgtacggcaac |
| | cgcgtgttcaccgagtacccccaggacatcgtcga |
| | ctacttcaagaactcctgccccgccggctacacct |
| | gggaccgctccttcctgttcgaggacggcgccgtg |
| | tgcatctgcaacgccgacatcaccgtgagcgtgga |
| | ggagaactgcatgtaccacgagtccaagttctacg |
| | gcgtgaacttccccgccgacggccccgtgatgaag |
| | aagatgaccgacaactgggagccctcctgcgagaa |
| | gatcatccccgtgcccaagcagggcatcttgaagg |
| | gcgacgtgagcatgtacctgctgctgaaggacggt |
| | ggccgcttgcgctgccagttcgacaccgtgtacaa |
| | ggccaagtccgtgccccgcaagatgcccgactggc |
| | acttcatccagcacaagctgacccgcgaggaccgc |
| | agcgacgccaagaaccagaagtggcacctgaccga |
| | gcacgccatcgcctccggctccgccttgccctaaa |
| | gatctacgggtggcatccctgtgacccctccccag |
| | tgcctctcctggccctggaagttgccactccagtg |
| | cccaccagccttgtcctaataaaattaagttgcat |
| | cattttgtctgactaggtgtccttctataatatta |
| | tggggtggaggggggtggtatggagcaaggggcaa |
| | gttgggaagacaacctgtagggcctgcggggtcta |
| | ttgggaaccaagctggagtgcagtggcacaatctt |
| | ggctcactgcaatctccgcctcctgggttcaagcg |
| | attctcctgcctcagcctcccgagttgttgggatt |
| | ccaggcatgcatgaccaggctcagctaatttttgt |
| | ttttttggtagagacggggtttcaccatattggcc |
| | aggctggtctccaactcctaatctcaggtgatcta |
| | cccaccttggcctcccaaattgctgggattacagg |
| | cgtgaaccactgctcccttccctgtccttaagagg |
| | aagagagagaccctcactgctggggagtccctgcc |
| | acactcagtcccccaccacactgaatctcccctcc |
| | tcacagttgccatgtagaccccttgaagaggggag |
| | gggcctagggagccgcaccttgtcatgtaccatca |
| | ataaagtaccctgtgctcaaccagttacttgtcct |
| | gtcttattctagggtctggggcagaggggagggaa |
| | gctgggcttgtgtcaaggtgagacattcttgctgg |
| | ggagggacctggtatgttctcctcagactgagggt |
| | agggcctccaaacagccttgcttgcttcgagaacc |
| | atttgcttcccgctcagacgtcttgagtgctacag |
| | gaagctggcaccactacttcagagaacaaggcctt |
| | ttcctctcctcgctccagtcctaggctatctgctg |
| | ttggccaaacatggaagaagctattctgtgggcag |
| | ccccagggaggctgacaggtggaggaagtcagggc |
| | tcgcactgggctctgacgctgactggttagtggag |
| | ctcagcctggagctgagctgcagcgggcaattcca |
| | gcttggcctccgcagctgtgaggtcttgagcacgt |
| | gctctattgctttctgtgccctcgtgtcttatctg |
| | aggacatcgtggccagcccctaaggtcttcaagca |
| | ggattcatctaggtaaaccaagtacctaaaaccat |
| | gcccaaggcggtaaggactatataatgtttaaaaa |
| | tcggtaaaaatgcccacctcgcatagttttgagga |
| | agatgaactgagatgtgtcagggt |
| | (SEQ ID NO: 6137) |
| dsODN_ | ggctgtgggcaaggtcatccctgagctgaacggga |
| 500HA | agctcactggcatggccttccgtgtccccactgcc |
| | aacgtgtcagtggtggacctgacctgccgtctaga |
| | aaaacctgccaaatatgatgacatcaagaaggtgg |
| | tgaagcaggcgtcggagggccccctcaagggcatc |
| | ctgggctacactgagcaccaggtggtctcctctga |
| | cttcaacagcgacacccactcctccacctttgacg |
| | ctggggctggcattgccctcaacgaccactttgtc |
| | aagctcatttcctggtatgtggctggggccagaga |
| | ctggctcttaaaaagtgcagggtctggcgccctct |
| | ggtggctggctcagaaaaagggccctgacaactct |
| | tttcatcttctaggtatgacaacgaatttggctac |
| | agcaacagggtggtggacctcatggcccacatggc |
| | ctccaaggagtaagacccctggaccaccagcccca |
| | gcaagagcacACGTTACTGGCCGAAGCCGCTTGGA |
| | ATAAGGCCGGTGTGCGTTTGTCTATATGTTATTTT |
| | CCACCATATTGCCGTCTTTTGGCAATGTGAGGGCC |
| | CGGAAACCTGGCCCTGTCTTCTTGACGAGCATTCC |
| | TAGGGGTCTTTCCCCTCTCGCCAAAGGAATGCAAG |
| | GTCTGTTGAATGTCGTGAAGGAAGCAGTTCCTCTG |
| | GAAGCTTCTTGAAGACAAACAACGTCTGTAGCGAC |
| | CCTTTGCAGGCAGCGGAACCCCCCACCTGGCGACA |
| | GGTGCCTCTGCGGCCAAAAGCCACGTGTATAAGAT |
| | ACACCTGCAAAGGCGGCACAACCCCAGTGCCACGT |
| | TGTGAGTTGGATAGTTGTGGAAAGAGTCAAATGGC |
| | TCTCCTCAAGCGTATTCAACAAGGGGCTGAAGGAT |
| | GCCCAGAAGGTACCCCATTGTATGGGATCTGATCT |
| | GGGGCCTCGGTGCACATGCTTTACATGTGTTTAGT |
| | CGAGGTTAAAAAAACGTCTAGGCCCCCCGAACCAC |
| | GGGGACGTGGTTTTCCTTTGAAAAACACGATGATA |
| | ATAATGGCCCAGTCCAAGCACGGCCTGACCAAGGA |
| | GATGACCATGAAGTACCGCATGGAGGGCTGCGTGG |
| | ACGGCCACAAGTTCGTGATCACCGGCGAGGGCATC |
| | GGCTACCCCTTCAAGGGCAAGCAGGCCATCAACCT |
| | GTGCGTGGTGGAGGGCGGCCCCTTGCCCTTCGCCG |
| | AGGACATCTTGTCCGCCGCCTTCATGTACGGCAAC |
| | CGCGTGTTCACCGAGTACCCCCAGGACATCGTCGA |
| | CTACTTCAAGAACTCCTGCCCCGCCGGCTACACCT |
| | GGGACCGCTCCTTCCTGTTCGAGGACGGCGCCGTG |
| | TGCATCTGCAACGCCGACATCACCGTGAGCGTGGA |
| | GGAGAACTGCATGTACCACGAGTCCAAGTTCTACG |
| | GCGTGAACTTCCCCGCCGACGGCCCCGTGATGAAG |
| | AAGATGACCGACAACTGGGAGCCCTCCTGCGAGAA |
| | GATCATCCCCGTGCCCAAGCAGGGCATCTTGAAGG |
| | GCGACGTGAGCATGTACCTGCTGCTGAAGGACGGT |
| | GGCCGCTTGCGCTGCCAGTTCGACACCGTGTACAA |
| | GGCCAAGTCCGTGCCCCGCAAGATGCCCGACTGGC |
| | ACTTCATCCAGCACAAGCTGACCCGCGAGGACCGC |
| | AGCGACGCCAAGAACCAGAAGTGGCACCTGACCGA |
| | GCACGCCATCGCCTCCGGCTCCGCCTTGCCCTAAA |
| | GATCTACgggtggcatccctgtgacccctccccag |
| | tgcctctcctggccctggaagttgccactccagtg |
| | cccaccagccttgtcctaataaaattaagttgcat |
| | cattttgtctgactaggtgtccttctataatatta |
| | tggggtggaggggggtggtatggagcaaggggcaa |
| | gttgggaagacaacctgtagggcctgcggggtcta |
| | ttgggaaccaagctggagtgcagtggcacaatctt |
| | ggctcactgcaatctccgcctcctgggttcaagcg |
| | attctcctgcctcagcctcccgagttgttgggatt |
| | ccaggcatgcatgaccaggctcagctaatttttgt |
| | ttttttggtagagacggggtttcaccatattggcc |
| | aggctggtctccaactcctaatctcaggtgatcta |
| | cccaccttggcctcccaaattgctgggattacagg |
| | cgtgaaccactgctcccttccctgtccttaagagg |
| | aagagagagaccctcactgctggggagtccctgcc |
| | acactcagtcccccaccacactgaatctcccctcc |
| | tcacagttgccatgtagaccccttgaagaggggag |
| | gggcctagggagccgcaccttgtcatgtaccatca |
| | ataaagtaccctgtgctcaaccagttacttgtcct |
| | gtcttattctagggtctggggcagaggggagggaa |
| | gctgggcttgtgtcaaggtgagacattcttgctgg |
| | ggagggacctggtatgttctcctcagactgagggt |
| | agggcctccaaacagccttgcttgcttcgagaacc |
| | atttgcttcccgctcagacgtcttgagtgctacag |
| | gaagctggcaccactacttcagagaacaaggcctt |
| | ttcctctcctcgctccagtcctaggctatctgctg |
| | ttggccaaacatggaagaagctattctgtgggcag |
| | ccccagggaggctgacaggtggaggaagtcagggc |
| | tcgc (SEQ ID NO: 6138) |

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A system for altering a target sequence, comprising a modified single-guide RNA (sgRNA) and a donor DNA, wherein the modified sgRNA comprises a CRISPR RNA (crRNA) and a trans-active RNA (tracrRNA), wherein the modified sgRNA comprises one or more internal anchors that are at least 5 nucleotides away from both 3′ and 5′ ends of the modified sgRNA, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI).

2. The system of claim 1, wherein the non-covalent bond is a Watson-Crick interaction.

3. The system of claim 1, wherein the modified sgRNA comprises a nexus, a first hairpin, and a single-stranded region between the tracrRNA and the crRNA, optionally the modified sgRNA further comprises a bulge region,
preferably, the modified sgRNA further comprises a second hairpin.

4-5. (canceled)

6. The system of one claim 1, wherein the internal anchor of the one or more internal anchors is located in a single-stranded region of the modified sgRNA, optionally the internal anchor of the one or more internal anchors is located in the single-stranded region between the tracrRNA and the crRNA;
and/or, the internal anchor of the one or more internal anchors is located in a single-stranded region within the first hairpin;
and/or, the internal anchor of the one or more internal anchors is located in a single-stranded region between the nexus and the first hairpin;
and/or, the internal anchor of the one or more internal anchors is located in a single-stranded region within the second hairpin.

7-10. (canceled)

11. The system of claim 1, wherein each of the one or more internal anchors or each of the one or more binding segments is 3-nucleotide to 100-nucleotide long; preferably 3-nucleotide to 20-nucleotide long; more preferably about 5-nucleotide long;
and/or, each of the one or more internal anchors comprises a sequence from SEQ ID NOs: 1 to 472 from Table 1;
optionally,
each of the one or more internal anchors comprises a sequence from SEQ ID NOs: 473 to 3056 from Table 2; or, each of the one or more binding segments comprises a sequence from SEQ ID NO. 3057 to 3528 from Table 3; or, each of the one or more binding segments comprises a sequence from SEQ ID NO. 3529 to 6112 from Table 4.

12-17. (canceled)

18. The system of claim 1, wherein the one or more binding segments are linked by a linker; preferably, the linker is a sequence of poly-deoxyadenosines.

19-21. (canceled)

22. The system of any claim 1, wherein the SOI comprises the target sequence with one or more nucleotide substitution, one or more nucleotide insertion, one or more nucleotide deletion, or any combination thereof;
preferably, the one or more nucleotide insertion comprises 1 to 100 nucleotides, 101 to 1000 nucleotides, 1001 to 10,000 nucleotides, or 10,001 to 100,000 nucleotides; and/or, the one or more nucleotide deletion comprises 1 to 100 nucleotides, 101 to 1000 nucleotides, 1001 to 10,000 nucleotides, or 10,001 to 100,000 nucleotides, or 1 to 50 nucleotides;
more preferably, the one or more nucleotide insertion comprises 2 to 10 random nucleotides.

23-25. (canceled)

26. The system of any claim 1, wherein the second portion of the donor DNA further comprises an upstream and/or a downstream homology arm; preferably, the upstream homology arm is 5 to 1000-nucleotide long; optionally, the downstream homology arm is about 10 to 1000-nucleotide long;
and/or, the first portion of the donor DNA is at 5′ of the second portion of the donor DNA, or at 3′ of the second portion of the donor DNA;
and/or, the donor DNA is single-stranded, or the first portion of the donor DNA is single-stranded and the second portion of the donor DNA is fully or partially double-stranded;
optionally, the donor DNA is close ended on 3′ and/or 5′ end.

27-33. (canceled)

34. The system of any claim 1, wherein the system further comprises a CRISPR nuclease.

35-36. (canceled)

37. A system comprising a donor DNA and two modified single-guide RNAs (sgRNAs) for cutting at a first locus on a first chromosome and a second locus on a second chromosome, wherein each of the modified sgRNAs comprises a CRISPR RNA (crRNA) and a trans-active RNA (tracrRNA), wherein each of the modified sgRNAs comprises one or more internal anchors that are at least 5 nucleotides away from both 3′ and 5′ ends of each of the modified sgRNAs, wherein the donor DNA comprises a first portion and a second portion, wherein the first portion comprises one or more binding segments capable of binding to an internal anchor of the one or more internal anchors via a non-covalent bond and the second portion comprises a sequence of interest (SOI), wherein the donor DNA comprises an upstream homology arm and/or a downstream homology arm.

38. The system of claim 37, wherein the first chromosome and the second chromosome are the same;
and/or, the first locus is at 5′ of the second locus; or, the first chromosome and the second chromosome are different;
and/or, the first locus and the second locus are at least 50, 100, 1,000, 10,000, or 100,000 nucleotides apart.

39-41. (canceled)

42. The system of claim 37, wherein the upstream homology arm flanks 5′ end of the first locus, and/or, the downstream homology arm flanks 3′ end of the second locus;
and/or, the non-covalent bond is a Watson-Crick interaction.

43-44. (canceled)

45. The system of claim 37, wherein the modified sgRNA comprises a nexus, a first hairpin, and a single-stranded region between the tracrRNA and the crRNA;
optionally the modified sgRNA further comprises a bulge region; and/or, the modified sgRNA further comprises a second hairpin;
and/or, the internal anchor of the one or more internal anchors is located in a single-stranded region of the modified sgRNA;
and/or, the internal anchor of the one or more internal anchors is located in the single-stranded region between the tracrRNA and the crRNA;
and/or, the internal anchor of the one or more internal anchors is located in a single-stranded region within the first hairpin;
and/or, the internal anchor of the one or more internal anchors is located in a single-stranded region between the nexus and the first hairpin.

46-51. (canceled)

52. The system of claim 37, wherein the modified sgRNA further comprises a second hairpin, and wherein the internal anchor of the one or more internal anchors is located in a single-stranded region within the second hairpin; optionally, each of the one or more internal anchors or each of the one or more binding segments is 3-nucleotide to 100-nucleotide long;
and/or, each of the one or more internal anchors comprises a sequence from SEQ ID NOs: 1 to 472 from Table 1; or, each of the one or more internal anchors comprises a sequence from SEQ ID NOs: 473 to 3056 from Table 2; or, each of the one or more binding segments comprises a sequence from SEQ ID NO. 3057 to 3528 from Table 3;
or, each of the one or more binding segments comprises a sequence from SEQ ID NO. 3529 to 6112 from Table 4.

53-59. (canceled)

60. The system of claim 37, wherein the one or more binding segments are linked by a linker; preferably the linker is a sequence of poly-deoxyadenosines;
and/or, the SOI comprises a region between the first locus and the second locus with one or more nucleotide substitution, one or more nucleotide insertion, one or more nucleotide deletion, or any combination thereof.

61-66. (canceled)

67. The system of claim 37, wherein the upstream homology arm is 5 to 1000-nucleotide long; optionally, the downstream homology arm is about 10 to 1000-nucleotide long;
and/or, the first portion of the donor DNA is at 5′ of the second portion of the donor DNA; or, the first portion of the donor DNA is at 3′ of the second portion of the donor DNA;
and/or, the donor DNA is single-stranded; or, the first portion of the donor DNA is single-stranded and the second portion of the donor DNA is fully or partially double-stranded;
optionally the donor DNA is close ended on 3′ and/or 5′ end;
and/or, the system further comprises a CRISPR nuclease.

68-76. (canceled)

77. A method of modifying a cell, wherein the method comprises transporting a system of claim 1 into the cell.

78. The method of claim 77, wherein the transporting comprises:
a. incubating the CRISPR nuclease and the modified sgRNA to form a ribonucleoprotein (RNP) complex;
b. applying the donor DNA to the RNP complex; and
c. delivering the RNP complex-donor DNA from (b) to the cell.

79. The method of claim 78, wherein in step (a) the ratio of the CRISPR nuclease and the modified sgRNA is about 1:0.5 to about 1:10; or, in step (a), the ratio of the CRISPR nuclease and the modified sgRNA is about 1:1 to 1:2.

80. (canceled)

81. The method of claim 77, wherein the transporting comprises:
a. providing one or more vectors comprising a nucleotide sequence encoding the CRISPR nuclease and a nucleotide sequence encoding the modified gRNA;
b. delivering the one or more vectors of (a) to the cell; and
c. delivering the donor DNA to the cell,
preferably, step (c) is performed about 6 to 48 hours after step (b).

82. (canceled)

83. The method of claim 77, wherein the delivering is achieved by viral vectors, liposomes, lipid nanoparticles, or electroporation;
and/or, the cell is an immune cell; preferably the immune cell is a T cell, a B cell, an NK cell, or a hematopoietic stem cell;
and/or, the method is performed ex vivo or in vivo;
and/or, the method has a percentage of desired editing is at least 10%, at least 50%, at least 100%, or at least 200% higher than a comparable system without the donor DNA comprising the first portion that binds to the modified sgRNA and/or without the modified sgRNA with the one or more internal anchors;
and/or, the method has a translocation, large insertion, or large deletion rate at least 10%, at least 50%, or at least 100% lower than a comparable system without the donor DNA comprising the first portion that binds to the modified sgRNA and/or without the modified sgRNA with the one or more internal anchors.

84-89. (canceled)

90. A method of treating a genetic disorder, wherein the method comprises administering to a subject in need thereof with an effective amount of the system of claim 1.

91. (canceled)

92. A method of treating a genetic disorder, wherein the method comprises administering to a subject in need thereof with an effective amount of the system of claim 37.

93. A method of modifying a cell, wherein the method comprises transporting a system of claim 37 into the cell.

94. The method of claim 93, wherein the transporting comprises:
a. incubating the CRISPR nuclease and the modified sgRNA to form a ribonucleoprotein (RNP) complex;
b. applying the donor DNA to the RNP complex; and
c. delivering the RNP complex-donor DNA from (b) to the cell.

95. The method of claim 93, wherein in step (a) the ratio of the CRISPR nuclease and the modified sgRNA is about 1:0.5 to about 1:10; or, in step (a), the ratio of the CRISPR nuclease and the modified sgRNA is about 1:1 to 1:2.

96. The method of claim 93, wherein the transporting comprises:
a. providing one or more vectors comprising a nucleotide sequence encoding the CRISPR nuclease and a nucleotide sequence encoding the modified gRNA;
b. delivering the one or more vectors of (a) to the cell; and
c. delivering the donor DNA to the cell,
preferably, step (c) is performed about 6 to 48 hours after step (b).

97. The method of claim 93, wherein the delivering is achieved by viral vectors, liposomes, lipid nanoparticles, or electroporation;
and/or, the cell is an immune cell; preferably the immune cell is a T cell, a B cell, an NK cell, or a hematopoietic stem cell;
and/or, the method is performed ex vivo or in vivo;
and/or, the method has a percentage of desired editing is at least 10%, at least 50%, at least 100%, or at least 200% higher than a comparable system without the donor DNA comprising the first portion that binds to the modified sgRNA and/or without the modified sgRNA with the one or more internal anchors;
and/or, the method has a translocation, large insertion, or large deletion rate at least 10%, at least 50%, or at least 100% lower than a comparable system without the donor DNA comprising the first portion that binds to the modified sgRNA and/or without the modified sgRNA with the one or more internal anchors.

98. A method of treating a genetic disorder, wherein the method comprises administering to a subject in need thereof with an effective amount of the system of claim 37.


SEQUENCE LISTING (6272 sequences)

SEQ ID NO: 1-6112 - skipped (6112 entries)

SEQ ID NO: 6113 - DNA, 100 bp, synthetic construct
  source 1..100: mol_type = other DNA
  misc_feature 1..67: note = RNA
  misc_feature 68..100: note = DNA
agcatagcaagttaaaataaggctagtccgttatcaacttgaaaaagtggcaccgagtcggtgctttctttcctctgccatcaaagcgtgctcagtctgg

SEQ ID NO: 6114 - DNA, 103 bp, synthetic construct
  source 1..103: mol_type = other DNA
  misc_feature 1..67: note = RNA
  misc_feature 68..103: note = DNA
agcatagcaagttaaaataaggctagtccgttatcaacttgaaaaagtggcaccgagtcggtgctttctttcctctgccatcaaagcgtgctcagtctgggcc

SEQ ID NO: 6115 - DNA, 106 bp, synthetic construct
  source 1..106: mol_type = other DNA
  misc_feature 1..67: note = RNA
  misc_feature 68..106: note = DNA
agcatagcaagttaaaataaggctagtccgttatcaacttgaaaaagtggcaccgagtcggtgctttctttcctctgccatcaaagcgtgctcagtctgggcccca

SEQ ID NO: 6116 - DNA, 109 bp, synthetic construct
  source 1..109: mol_type = other DNA
  misc_feature 1..67: note = RNA
  misc_feature 68..109: note = DNA
agcatagcaagttaaaataaggctagtccgttatcaacttgaaaaagtggcaccgagtcggtgctttctttcctctgccatcaaagcgtgctcagtctgggccccaagg

SEQ ID NO: 6117 - DNA, 39 bp, synthetic construct
  source 1..39: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
ctttcctctgccatcaaagcgtgctcagtctgggcccca

SEQ ID NO: 6118 - DNA, 42 bp, synthetic construct
  source 1..42: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 40: mod_base = OTHER; note = nucleotide with phosphorothioate linkage
  modified_base 41: mod_base = OTHER; note = nucleotide with phosphorothioate linkage
ccactttcctctgccatcaaagcgtgctcagtctgggcccca

SEQ ID NO: 6119 - DNA, 44 bp, synthetic construct
  source 1..44: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 42: mod_base = OTHER; note = nucleotide with phosphorothioate linkage
  modified_base 43: mod_base = OTHER; note = nucleotide with phosphorothioate linkage
ctccactttcctctgccatcaaagcgtgctcagtctgggcccca

SEQ ID NO: 6120 - DNA, 46 bp, synthetic construct
  source 1..46: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 44: mod_base = OTHER; note = nucleotide with phosphorothioate linkage
  modified_base 45: mod_base = OTHER; note = nucleotide with phosphorothioate linkage
tcctccactttcctctgccatcaaagcgtgctcagtctgggcccca

SEQ ID NO: 6121 - DNA, 49 bp, synthetic construct
  source 1..49: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 47: mod_base = OTHER; note = nucleotide with phosphorothioate linkage
  modified_base 48: mod_base = OTHER; note = nucleotide with phosphorothioate linkage
gtgtcctccactttcctctgccatcaaagcgtgctcagtctgggcccca

SEQ ID NO: 6122 - DNA, 52 bp, synthetic construct
  source 1..52: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 50: mod_base = OTHER; note = nucleotide with phosphorothioate linkage
  modified_base 51: mod_base = OTHER; note = nucleotide with phosphorothioate linkage
gtcgtgtcctccactttcctctgccatcaaagcgtgctcagtctgggcccca

SEQ ID NO: 6123 - DNA, 15 bp, synthetic construct
  source 1..15: mol_type = other DNA
acattgttctcactt

SEQ ID NO: 6124 - RNA, 101 nt, synthetic construct
  source 1..101: mol_type = other RNA
ggcccagactgagcacgtgagttttagagctagtggagaaatagcaagttaaaataaggctagtccgttatcaacttgaaaaagtggcaccgagtcggtgc

SEQ ID NO: 6125 - RNA, 101 nt, synthetic construct
  source 1..101: mol_type = other RNA
tgatcacctgcccaaatgtggttttagagctagtggagaaatagcaagttaaaataaggctagtccgttatcaacttgaaaaagtggcaccgagtcggtgc

SEQ ID NO: 6126 - RNA, 101 nt, synthetic construct
  source 1..101: mol_type = other RNA
ctgccatcacgtgctcagtcgttttagagctagtggagaaatagcaagttaaaataaggctagtccgttatcaacttgaaaaagtggcaccgagtcggtgc

SEQ ID NO: 6127 - RNA, 100 nt, synthetic construct
  source 1..100: mol_type = other RNA
  modified_base 16: mod_base = OTHER; note = Thymine
tgggcaggtgatcaatgctgttttagagctagtggagaaatagcaagttaaaataaggctagtccgttatcaacttgaaaaagtggcaccgagtcggtgc

SEQ ID NO: 6128 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccaggagatgctccctaagcattgatcacctgcccaaatcgtgctcagtctgggccccaaggattgacccaggcc

SEQ ID NO: 6129 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccaggcctgggtcaatccttggggcccagactgagcacgatttgggcaggtgatcaatgcttagggagcatctcc

SEQ ID NO: 6130 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccaccagccctggcctgggtcaatccttggggcccagacatttgggcaggtgatcaatgcttagggagcatctcc

SEQ ID NO: 6131 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccacaaatcctcgcatttgggcaggagatgctccctaagcgtgctcagtctgggccccaaggattgacccaggcc

SEQ ID NO: 6132 - RNA, 101 nt, synthetic construct
  source 1..101: mol_type = other RNA
gcctgcaaactggtaggcgcgttttagagctagtggagaaatagcaagttaaaataaggctagtccgttatcaacttgaaaaagtggcaccgagtcggtgc

SEQ ID NO: 6133 - RNA, 101 nt, synthetic construct
  source 1..101: mol_type = other RNA
aagcatgatcagaacggttggttttagagctagtggagaaatagcaagttaaaataaggctagtccgttatcaacttgaaaaagtggcaccgagtcggtgc

SEQ ID NO: 6134 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccagggttttgactattttagcaagcatgatcagaacggcctaccagtttgcaggctcactaggtagccgtggga

SEQ ID NO: 6135 - DNA, 77 bp, synthetic construct
  source 1..77: mol_type = other DNA
  modified_base 1: mod_base = OTHER; note = nucleotide with phosphorothioate linkage
  modified_base 2: mod_base = OTHER; note = nucleotide with phosphorothioate linkage
  modified_base 75: mod_base = OTHER; note = nucleotide with phosphorothioate linkage
  modified_base 76: mod_base = OTHER; note = nucleotide with phosphorothioate linkage
ctccatcccacggctacctagtgagcctgcaaactggtaggccgttctgatcatgcttgctaaaatagtcaaaaccc

SEQ ID NO: 6136 - RNA, 111 nt, synthetic construct
  source 1..111: mol_type = other RNA
agccccagcaagagcacaaggttttagagctagaagtgagaacaatgtaaatagcaagttaaaataaggctagtccgttatcaacttgaaaaagtggcaccgagtcggtgc

SEQ ID NO: 6137 - DNA, 3349 bp, synthetic construct
  source 1..3349: mol_type = other DNA
acattgttctcacttgggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaggagtaagacccctggaccaccagccccagcaagagcacacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataataatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagttttgaggaagatgaactgagatgtgtcagggt

SEQ ID NO: 6138 - DNA, 2734 bp, synthetic construct
  source 1..2734: mol_type = other DNA
ggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaggagtaagacccctggaccaccagccccagcaagagcacacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataataatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgc

SEQ ID NO: 6139 - DNA, 96 bp, synthetic construct
  source 1..96: mol_type = other DNA
tggggcccagactgagcacgctttgatggcagaggaaagaaaaaaaactccaaaaaactttcctctgccatcaaagcgtgctcagtctgggcccca

SEQ ID NO: 6140 - DNA, 107 bp, synthetic construct
  source 1..107: mol_type = other DNA
  misc_feature 1..107: note = cyclic
tggggcccagactgagcacgctttgatggcagaggaaagaaaaaaaactccaaaaaactttcctctgccatcaaagcgtgctcagtctgggccccaaaaaaaaaaaa

SEQ ID NO: 6141 - DNA, 57 bp, synthetic construct
  source 1..57: mol_type = other DNA
  misc_feature 1..57: note = cyclic
aaaaaaaactccaaaaaactttcctctgccatcaaagcgtgctcagtctgggcccca

SEQ ID NO: 6142 - DNA, 78 bp, synthetic construct
  source 1..78: mol_type = other DNA
gaaagaaaaaaaactccaaaaaactttcctctgccatcaaagcgtgctcagtctgggccccaaaaaaaaaaaatgggg

SEQ ID NO: 6143 - DNA, 23 bp, unidentified
  source 1..23: mol_type = unassigned DNA
  misc_difference 21: note = a, c, t, g, unknown or other
ggcccagactgagcacgtgangg

SEQ ID NO: 6144 - DNA, 23 bp, synthetic construct
  source 1..23: mol_type = other DNA
ggcccagactgagcacgtgatgg

SEQ ID NO: 6145 - DNA, 23 bp, synthetic construct
  source 1..23: mol_type = other DNA
gacacagaccgggcacgtgaggg

SEQ ID NO: 6146 - DNA, 23 bp, unidentified
  source 1..23: mol_type = unassigned DNA
  misc_difference 21: note = a, c, t, g, unknown or other
ggcccagactgagcacgtgangg

SEQ ID NO: 6147 - DNA, 23 bp, synthetic construct
  source 1..23: mol_type = other DNA
ggcccagactgagcacgtgatgg

SEQ ID NO: 6148 - DNA, 23 bp, synthetic construct
  source 1..23: mol_type = other DNA
gacacagaccgggcacgtgaggg

SEQ ID NO: 6149 - DNA, 23 bp, unidentified
  source 1..23: mol_type = unassigned DNA
  misc_difference 21: note = a, c, t, g, unknown or other
ggcccagactgagcacgtgangg

SEQ ID NO: 6150 - DNA, 23 bp, synthetic construct
  source 1..23: mol_type = other DNA
ggcccagactgagcacgtgatgg

SEQ ID NO: 6151 - DNA, 23 bp, synthetic construct
  source 1..23: mol_type = other DNA
gacacagaccgggcacgtgaggg

SEQ ID NO: 6152 - DNA, 23 bp, synthetic construct
  source 1..23: mol_type = other DNA
cacccagactgagcacgtgctgg

SEQ ID NO: 6153 - DNA, 23 bp, unidentified
  source 1..23: mol_type = unassigned DNA
  misc_difference 21: note = a, c, t, g, unknown or other
ggcccagactgagcacgtgangg

SEQ ID NO: 6154 - DNA, 23 bp, synthetic construct
  source 1..23: mol_type = other DNA
ggcccagactgagcacgtgatgg

SEQ ID NO: 6155 - DNA, 23 bp, synthetic construct
  source 1..23: mol_type = other DNA
gacacagaccgggcacgtgaggg

SEQ ID NO: 6156 - DNA, 23 bp, unidentified
  source 1..23: mol_type = unassigned DNA
  misc_difference 21: note = a, c, t, g, unknown or other
ggcccagactgagcacgtgangg

SEQ ID NO: 6157 - DNA, 23 bp, synthetic construct
  source 1..23: mol_type = other DNA
ggcccagactgagcacgtgatgg

SEQ ID NO: 6158 - DNA, 23 bp, synthetic construct
  source 1..23: mol_type = other DNA
agctcagactgagcaagtgaggg

SEQ ID NO: 6159 - DNA, 23 bp, unidentified
  source 1..23: mol_type = unassigned DNA
  misc_difference 21: note = a, c, t, g, unknown or other
ggcccagactgagcacgtgangg

SEQ ID NO: 6160 - DNA, 23 bp, synthetic construct
  source 1..23: mol_type = other DNA
ggcccagactgagcacgtgatgg

SEQ ID NO: 6161 - DNA, 23 bp, synthetic construct
  source 1..23: mol_type = other DNA
agcagagtctgatcacctgactg

SEQ ID NO: 6162 - DNA, 23 bp, synthetic construct
  source 1..23: mol_type = other DNA
gacacagaccgggcacgtgaggg

SEQ ID NO: 6163 - DNA, 23 bp, synthetic construct
  source 1..23: mol_type = other DNA
cacccagactgagcacgtgctgg

SEQ ID NO: 6164 - DNA, 23 bp, unidentified
  source 1..23: mol_type = unassigned DNA
  misc_difference 21: note = a, c, t, g, unknown or other
ggcccagactgagcacgtgangg

SEQ ID NO: 6165 - DNA, 23 bp, synthetic construct
  source 1..23: mol_type = other DNA
ggcccagactgagcacgtgatgg

SEQ ID NO: 6166 - DNA, 23 bp, synthetic construct
  source 1..23: mol_type = other DNA
gtcccagactgcccattgggtgg

SEQ ID NO: 6167 - DNA, 23 bp, unidentified
  source 1..23: mol_type = unassigned DNA
  misc_difference 21: note = a, c, t, g, unknown or other
ggcccagactgagcacgtgangg

SEQ ID NO: 6168 - DNA, 23 bp, synthetic construct
  source 1..23: mol_type = other DNA
ggcccagactgagcacgtgatgg

SEQ ID NO: 6169 - DNA, 23 bp, synthetic construct
  source 1..23: mol_type = other DNA
gtcacagactgcccaggtgtaag

SEQ ID NO: 6170 - DNA, 23 bp, synthetic construct
  source 1..23: mol_type = other DNA
gggccagtctgagctcatcacca

SEQ ID NO: 6171 - DNA, 23 bp, synthetic construct
  source 1..23: mol_type = other DNA
gtcccagactgcccgcatgaata

SEQ ID NO: 6172 - DNA, 23 bp, unidentified
  source 1..23: mol_type = unassigned DNA
  misc_difference 21: note = a, c, t, g, unknown or other
ggcccagactgagcacgtgangg

SEQ ID NO: 6173 - DNA, 23 bp, synthetic construct
  source 1..23: mol_type = other DNA
ggcccagactgagcacgtgatgg

SEQ ID NO: 6174 - DNA, 23 bp, synthetic construct
  source 1..23: mol_type = other DNA
gacacagaccgggcacgtgaggg

SEQ ID NO: 6175 - DNA, 23 bp, synthetic construct
  source 1..23: mol_type = other DNA
cacccagactgagcacgtgctgg

SEQ ID NO: 6176 - DNA, 23 bp, unidentified
  source 1..23: mol_type = unassigned DNA
  misc_difference 21: note = a, c, t, g, unknown or other
ggcccagactgagcacgtgangg

SEQ ID NO: 6177 - DNA, 23 bp, synthetic construct
  source 1..23: mol_type = other DNA
ggcccagactgagcacgtgatgg

SEQ ID NO: 6178 - DNA, 23 bp, synthetic construct
  source 1..23: mol_type = other DNA
gacacagaccgggcacgtgaggg

SEQ ID NO: 6179 - DNA, 23 bp, synthetic construct
  source 1..23: mol_type = other DNA
cacccagactgagcacgtgctgg

SEQ ID NO: 6180 - DNA, 23 bp, unidentified
  source 1..23: mol_type = unassigned DNA
  misc_difference 21: note = a, c, t, g, unknown or other
ggcccagactgagcacgtgangg

SEQ ID NO: 6181 - DNA, 23 bp, synthetic construct
  source 1..23: mol_type = other DNA
ggcccagactgagcacgtgatgg

SEQ ID NO: 6182 - DNA, 23 bp, synthetic construct
  source 1..23: mol_type = other DNA
gacacagaccgggcacgtgaggg

SEQ ID NO: 6183 - DNA, 23 bp, synthetic construct
  source 1..23: mol_type = other DNA
cacccagactgagcacgtgctgg

SEQ ID NO: 6184 - DNA, 23 bp, synthetic construct
  source 1..23: mol_type = other DNA
agctcagactgagcaagtgaggg

SEQ ID NO: 6185 - DNA, 23 bp, synthetic construct
  source 1..23: mol_type = other DNA
ggcccagactagagccgtggcgc

SEQ ID NO: 6186 - DNA, 23 bp, unidentified
  source 1..23: mol_type = unassigned DNA
  misc_difference 21: note = a, c, t, g, unknown or other
ggcccagactgagcacgtgangg

SEQ ID NO: 6187 - DNA, 23 bp, synthetic construct
  source 1..23: mol_type = other DNA
ggcccagactgagcacgtgatgg

SEQ ID NO: 6188 - DNA, 23 bp, synthetic construct
  source 1..23: mol_type = other DNA
gacacagaccgggcacgtgaggg

SEQ ID NO: 6189 - DNA, 23 bp, synthetic construct
  source 1..23: mol_type = other DNA
cacccagactgagcacgtgctgg

SEQ ID NO: 6190 - DNA, 23 bp, unidentified
  source 1..23: mol_type = unassigned DNA
  misc_difference 21: note = a, c, t, g, unknown or other
ggcccagactgagcacgtgangg

SEQ ID NO: 6191 - DNA, 23 bp, synthetic construct
  source 1..23: mol_type = other DNA
ggcccagactgagcacgtgatgg

SEQ ID NO: 6192 - DNA, 23 bp, synthetic construct
  source 1..23: mol_type = other DNA
gacacagaccgggcacgtgaggg

SEQ ID NO: 6193 - DNA, 23 bp, unidentified
  source 1..23: mol_type = unassigned DNA
  misc_difference 21: note = a, c, t, g, unknown or other
ggcccagactgagcacgtgangg

SEQ ID NO: 6194 - DNA, 23 bp, synthetic construct
  source 1..23: mol_type = other DNA
ggcccagactgagcacgtgatgg

SEQ ID NO: 6195 - DNA, 23 bp, synthetic construct
  source 1..23: mol_type = other DNA
gacacagaccgggcacgtgaggg

SEQ ID NO: 6196 - DNA, 23 bp, synthetic construct
  source 1..23: mol_type = other DNA
cacccagactgagcacgtgctgg

SEQ ID NO: 6197 - DNA, 23 bp, unidentified
  source 1..23: mol_type = unassigned DNA
  misc_difference 21: note = a, c, t, g, unknown or other
ggcccagactgagcacgtgangg

SEQ ID NO: 6198 - DNA, 23 bp, synthetic construct
  source 1..23: mol_type = other DNA
ggcccagactgagcacgtgatgg

SEQ ID NO: 6199 - DNA, 23 bp, synthetic construct
  source 1..23: mol_type = other DNA
gacacagaccgggcacgtgaggg

SEQ ID NO: 6200 - DNA, 23 bp, unidentified
  source 1..23: mol_type = unassigned DNA
  misc_difference 21: note = a, c, t, g, unknown or other
ggcccagactgagcacgtgangg

SEQ ID NO: 6201 - DNA, 23 bp, synthetic construct
  source 1..23: mol_type = other DNA
ggcccagactgagcacgtgatgg

SEQ ID NO: 6202 - DNA, 23 bp, synthetic construct
  source 1..23: mol_type = other DNA
gacacagaccgggcacgtgaggg

SEQ ID NO: 6203 - DNA, 23 bp, synthetic construct
  source 1..23: mol_type = other DNA
cacccagactgagcacgtgctgg

SEQ ID NO: 6204 - DNA, 23 bp, synthetic construct
  source 1..23: mol_type = other DNA
agctcagactgagcaagtgaggg

SEQ ID NO: 6205 - DNA, 23 bp, unidentified
  source 1..23: mol_type = unassigned DNA
  misc_difference 21: note = a, c, t, g, unknown or other
ggcccagactgagcacgtgangg

SEQ ID NO: 6206 - DNA, 23 bp, synthetic construct
  source 1..23: mol_type = other DNA
ggcccagactgagcacgtgatgg

SEQ ID NO: 6207 - DNA, 23 bp, unidentified
  source 1..23: mol_type = unassigned DNA
  misc_difference 21: note = a, c, t, g, unknown or other
ggcccagactgagcacgtgangg

SEQ ID NO: 6208 - DNA, 23 bp, synthetic construct
  source 1..23: mol_type = other DNA
ggcccagactgagcacgtgatgg

SEQ ID NO: 6209 - DNA, 23 bp, unidentified
  source 1..23: mol_type = unassigned DNA
  misc_difference 21: note = a, c, t, g, unknown or other
ggcccagactgagcacgtgangg

SEQ ID NO: 6210 - DNA, 23 bp, synthetic construct
  source 1..23: mol_type = other DNA
ggcccagactgagcacgtgatgg

SEQ ID NO: 6211 - DNA, 23 bp, synthetic construct
  source 1..23: mol_type = other DNA
gggccagcctcaccaggagagga

SEQ ID NO: 6212 - DNA, 23 bp, unidentified
  source 1..23: mol_type = unassigned DNA
  misc_difference 21: note = a, c, t, g, unknown or other
ggcccagactgagcacgtgangg

SEQ ID NO: 6213 - DNA, 23 bp, synthetic construct
  source 1..23: mol_type = other DNA
ggcccagactgagcacgtgatgg

SEQ ID NO: 6214 - DNA, 23 bp, synthetic construct
  source 1..23: mol_type = other DNA
ttcccagagtggcccagtgaggg

SEQ ID NO: 6215 - DNA, 23 bp, unidentified
  source 1..23: mol_type = unassigned DNA
  misc_difference 21: note = a, c, t, g, unknown or other
ggcccagactgagcacgtgangg

SEQ ID NO: 6216 - DNA, 23 bp, synthetic construct
  source 1..23: mol_type = other DNA
ggcccagactgagcacgtgatgg

SEQ ID NO: 6217 - DNA, 23 bp, synthetic construct
  source 1..23: mol_type = other DNA
atcaccgactgagcatgtgagta

SEQ ID NO: 6218 - DNA, 23 bp, unidentified
  source 1..23: mol_type = unassigned DNA
  misc_difference 21: note = a, c, t, g, unknown or other
ggcccagactgagcacgtgangg

SEQ ID NO: 6219 - DNA, 23 bp, synthetic construct
  source 1..23: mol_type = other DNA
ggcccagactgagcacgtgatgg

SEQ ID NO: 6220 - DNA, 23 bp, unidentified
  source 1..23: mol_type = unassigned DNA
  misc_difference 21: note = a, c, t, g, unknown or other
ggcccagactgagcacgtgangg

SEQ ID NO: 6221 - DNA, 23 bp, synthetic construct
  source 1..23: mol_type = other DNA
ggcccagactgagcacgtgatgg

SEQ ID NO: 6222 - DNA, 23 bp, unidentified
  source 1..23: mol_type = unassigned DNA
  misc_difference 21: note = a, c, t, g, unknown or other
ggcccagactgagcacgtgangg

SEQ ID NO: 6223 - DNA, 23 bp, synthetic construct
  source 1..23: mol_type = other DNA
ggcccagactgagcacgtgatgg

SEQ ID NO: 6224 - DNA, 23 bp, unidentified
  source 1..23: mol_type = unassigned DNA
  misc_difference 21: note = a, c, t, g, unknown or other
ggcccagactgagcacgtgangg

SEQ ID NO: 6225 - DNA, 23 bp, synthetic construct
  source 1..23: mol_type = other DNA
ggcccagactgagcacgtgatgg

SEQ ID NO: 6226 - DNA, 23 bp, unidentified
  source 1..23: mol_type = unassigned DNA
  misc_difference 21: note = a, c, t, g, unknown or other
ggcccagactgagcacgtgangg

SEQ ID NO: 6227 - DNA, 23 bp, synthetic construct
  source 1..23: mol_type = other DNA
ggcccagactgagcacgtgatgg

SEQ ID NO: 6228 - DNA, 23 bp, unidentified
  source 1..23: mol_type = unassigned DNA
  misc_difference 21: note = a, c, t, g, unknown or other
ggcccagactgagcacgtgangg

SEQ ID NO: 6229 - DNA, 23 bp, synthetic construct
  source 1..23: mol_type = other DNA
ggcccagactgagcacgtgatgg

SEQ ID NO: 6230 - DNA, 23 bp, unidentified
  source 1..23: mol_type = unassigned DNA
  misc_difference 21: note = a, c, t, g, unknown or other
ggcccagactgagcacgtgangg

SEQ ID NO: 6231 - DNA, 23 bp, synthetic construct
  source 1..23: mol_type = other DNA
ggcccagactgagcacgtgatgg

SEQ ID NO: 6232 - DNA, 23 bp, unidentified
  source 1..23: mol_type = unassigned DNA
  misc_difference 21: note = a, c, t, g, unknown or other
ggcccagactgagcacgtgangg

SEQ ID NO: 6233 - DNA, 23 bp, synthetic construct
  source 1..23: mol_type = other DNA
ggcccagactgagcacgtgatgg

SEQ ID NO: 6234 - DNA, 23 bp, unidentified
  source 1..23: mol_type = unassigned DNA
  misc_difference 21: note = a, c, t, g, unknown or other
ggcccagactgagcacgtgangg

SEQ ID NO: 6235 - DNA, 23 bp, synthetic construct
  source 1..23: mol_type = other DNA
ggcccagactgagcacgtgatgg

SEQ ID NO: 6236 - DNA, 23 bp, synthetic construct
  source 1..23: mol_type = other DNA
tgcccagtctgggcacctaactc

SEQ ID NO: 6237 - DNA, 23 bp, unidentified
  source 1..23: mol_type = unassigned DNA
  misc_difference 21: note = a, c, t, g, unknown or other
ggcccagactgagcacgtgangg

SEQ ID NO: 6238 - DNA, 23 bp, synthetic construct
  source 1..23: mol_type = other DNA
ggcccagactgagcacgtgatgg

SEQ ID NO: 6239 - DNA, 23 bp, unidentified
  source 1..23: mol_type = unassigned DNA
  misc_difference 21: note = a, c, t, g, unknown or other
ggcccagactgagcacgtgangg

SEQ ID NO: 6240 - DNA, 23 bp, synthetic construct
  source 1..23: mol_type = other DNA
ggcccagactgagcacgtgatgg

SEQ ID NO: 6241 - DNA, 23 bp, unidentified
  source 1..23: mol_type = unassigned DNA
  misc_difference 21: note = a, c, t, g, unknown or other
ggcccagactgagcacgtgangg

SEQ ID NO: 6242 - DNA, 23 bp, synthetic construct
  source 1..23: mol_type = other DNA
ggcccagactgagcacgtgatgg

SEQ ID NO: 6243 - DNA, 23 bp, unidentified
  source 1..23: mol_type = unassigned DNA
  misc_difference 21: note = a, c, t, g, unknown or other
ggcccagactgagcacgtgangg

SEQ ID NO: 6244 - DNA, 23 bp, synthetic construct
  source 1..23: mol_type = other DNA
ggcccagactgagcacgtgatgg

SEQ ID NO: 6245 - DNA, 23 bp, unidentified
  source 1..23: mol_type = unassigned DNA
  misc_difference 21: note = a, c, t, g, unknown or other
ggcccagactgagcacgtgangg

SEQ ID NO: 6246 - DNA, 23 bp, synthetic construct
  source 1..23: mol_type = other DNA
ggcccagactgagcacgtgatgg

SEQ ID NO: 6247 - DNA, 23 bp, unidentified
  source 1..23: mol_type = unassigned DNA
  misc_difference 21: note = a, c, t, g, unknown or other
ggcccagactgagcacgtgangg

SEQ ID NO: 6248 - DNA, 23 bp, synthetic construct
  source 1..23: mol_type = other DNA
ggcccagactgagcacgtgatgg

SEQ ID NO: 6249 - DNA, 23 bp, unidentified
  source 1..23: mol_type = unassigned DNA
  misc_difference 21: note = a, c, t, g, unknown or other
ggcccagactgagcacgtgangg

SEQ ID NO: 6250 - DNA, 23 bp, synthetic construct
  source 1..23: mol_type = other DNA
ggcccagactgagcacgtgatgg

SEQ ID NO: 6251 - DNA, 23 bp, unidentified
  source 1..23: mol_type = unassigned DNA
  misc_difference 21: note = a, c, t, g, unknown or other
ggcccagactgagcacgtgangg

SEQ ID NO: 6252 - DNA, 23 bp, synthetic construct
  source 1..23: mol_type = other DNA
ggcccagactgagcacgtgatgg

SEQ ID NO: 6253 - DNA, 23 bp, unidentified
  source 1..23: mol_type = unassigned DNA
  misc_difference 21: note = a, c, t, g, unknown or other
ggcccagactgagcacgtgangg

SEQ ID NO: 6254 - DNA, 23 bp, synthetic construct
  source 1..23: mol_type = other DNA
ggcccagactgagcacgtgatgg

SEQ ID NO: 6255 - DNA, 23 bp, unidentified
  source 1..23: mol_type = unassigned DNA
  misc_difference 21: note = a, c, t, g, unknown or other
ggcccagactgagcacgtgangg

SEQ ID NO: 6256 - DNA, 23 bp, synthetic construct
  source 1..23: mol_type = other DNA
ggcccagactgagcacgtgatgg

SEQ ID NO: 6257 - DNA, 23 bp, unidentified
  source 1..23: mol_type = unassigned DNA
  misc_difference 21: note = a, c, t, g, unknown or other
ggcccagactgagcacgtgangg

SEQ ID NO: 6258 - DNA, 23 bp, synthetic construct
  source 1..23: mol_type = other DNA
ggcccagactgagcacgtgatgg

SEQ ID NO: 6259 - DNA, 23 bp, unidentified
  source 1..23: mol_type = unassigned DNA
  misc_difference 21: note = a, c, t, g, unknown or other
ggcccagactgagcacgtgangg

SEQ ID NO: 6260 - DNA, 23 bp, synthetic construct
  source 1..23: mol_type = other DNA
ggcccagactgagcacgtgatgg

SEQ ID NO: 6261 - DNA, 23 bp, unidentified
  source 1..23: mol_type = unassigned DNA
  misc_difference 21: note = a, c, t, g, unknown or other
ggcccagactgagcacgtgangg

SEQ ID NO: 6262 - DNA, 23 bp, synthetic construct
  source 1..23: mol_type = other DNA
ggcccagactgagcacgtgatgg

SEQ ID NO: 6263 - DNA, 23 bp, unidentified
  source 1..23: mol_type = unassigned DNA
  misc_difference 21: note = a, c, t, g, unknown or other
ggcccagactgagcacgtgangg

SEQ ID NO: 6264 - DNA, 23 bp, synthetic construct
  source 1..23: mol_type = other DNA
ggcccagactgagcacgtgatgg

SEQ ID NO: 6265 - DNA, 23 bp, unidentified
  source 1..23: mol_type = unassigned DNA
  misc_difference 21: note = a, c, t, g, unknown or other
ggcccagactgagcacgtgangg

SEQ ID NO: 6266 - DNA, 23 bp, synthetic construct
  source 1..23: mol_type = other DNA
ggcccagactgagcacgtgatgg

SEQ ID NO: 6267 - DNA, 23 bp, unidentified
  source 1..23: mol_type = unassigned DNA
  misc_difference 21: note = a, c, t, g, unknown or other
ggcccagactgagcacgtgangg

SEQ ID NO: 6268 - DNA, 23 bp, synthetic construct
  source 1..23: mol_type = other DNA
ggcccagactgagcacgtgatgg

SEQ ID NO: 6269 - DNA, 86 bp, synthetic construct
  source 1..86: mol_type = other DNA
gctgcaagtaagcatgcatttgtaggcttgatgctttttttctgcttctccagccctggcctgggtcaatccttggggcccagact

SEQ ID NO: 6270 - DNA, 97 bp, synthetic construct
  source 1..97: mol_type = other DNA
aggtgatcaatgcttagggagcatctcctgcccaaatgcgaggatttgagagggacagtcgcctatacaggttcacagaactgactggcaggaccaa

SEQ ID NO: 6271 - DNA, 80 bp, synthetic construct
  source 1..80: mol_type = other DNA
tcgctgggggagcctcggcttcttctgggagaaaattcccacggctacctagtgagcctgcaaactggtaggcgccggcg

SEQ ID NO: 6272 - DNA, 67 bp, synthetic construct
  source 1..67: mol_type = other DNA
tccctcctcaaccgttctgatcatgcttgctaaaatagtcaaaaccccggccagttaaatatgcttt